(19) United States
(12) Patent Application Publication
(10) Pub. No.: US 2025/0308628 A1
(43) Pub. Date: Oct. 2, 2025

(54) METHYLATION AND AGING

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Guanhua Zhu, Ma On Shan (CN)

(21) Appl. No.: 19/096,399

(22) Filed: Mar. 31, 2025

Related U.S. Application Data
(60) Provisional application No. 63/572,164, filed on Mar. 29, 2024.

(51) Int. Cl. G16B 20/00; G16B 30/10; G16B 40/20; G16B 40/30; G16H 50/20

(52) U.S. Cl. CPC G16B 20/00; G16B 30/10; G16B 40/20; G16B 40/30; G16H 50/20

(57) ABSTRACT

Systems and method as described herein may determine and use methylation levels associated with various tissues and samples. For example, a method may include receiving sequence reads including methylation statuses at sites of cell-free DNA molecules. The method may further include aligning the sequence reads to N sets of one or more CpG sites or genes. Then, for each set of the N sets of one or more CpG sites or genes, the method may include identifying a group of sequence reads aligning to the set of one or more CpG sites or genes and determining a methylation level using the methylation statuses of the group of sequence reads.

### CROSS REFERENCE TO RELATED APPLICATIONS

This application claims the benefit of, and priority to, U.S. Provisional Application No. 63/572,164, filed on Mar. 29, 2024, which is hereby incorporated by reference in its entirety for all purposes.

### BACKGROUND

Ageing often refers to progressive physiological changes in an organism that may occur with the lapse of time from the birth of that organism (i.e. chronological age). The physiological changes may lead to senescence, a decline of biological functions, and/or a decline in an organism's ability to adapt to metabolic stress. The metabolic stress can be driven by metabolic disturbances which are influenced by environmental factors such as pathogens, temperature, noise, toxins, nutrient stress (excess or deficiency), oxidative stress, and hypoxia. Ageing is a leading cause of disease and disability. Chronological age can be a risk factor for many diseases in the human population, such as cardiovascular diseases, diabetes, cancer, Alzheimer's disease, and dementia (Partridge et al., 2018). However, predictive power for a certain disease (e.g., Alzheimer's disease, cancers, cardiovascular diseases, etc.) directly based on chronological age can be low (Lowsky et al., 2014). Therefore, it would be beneficial to have improved techniques.

### BRIEF SUMMARY

Embodiments provide systems, methods, and apparatuses for determining and using methylation levels associated with various tissues and samples. Examples are provided. A methylation level can be deduced based on methylation statuses of sites of plasma cell-free DNA molecules (or other samples with cell-free DNA, e.g., urine, saliva, genital washings). The methylation level can correspond to a CpG site or a gene and can be indicative of a biological age of a subject or of a particular tissue. Thus, one or more machine learning models can be trained to predict biological age based on one or more methylation levels for one or more CpG sites or one or more genes. The machine learning models may be trained using methylation level data for healthy subjects of various ages.

Additionally, a comparison of biological age to chronological age of a subject can be used to detect a presence of a disorder in the subject. For example, age acceleration for the subject or for a particular tissue can be detected based on the comparison. A level of age acceleration can be used to classify the presence of a disorder. When the presence of a disorder is detected, the particular disorder may be ascertained based on the particular tissue exhibiting age acceleration or based on one or more CpG sites or one or more genes with methylation levels indicative of the age acceleration.

In one embodiment, a method for measuring a biological age of a subject is provided. The method may be performed by a computer system receiving sequence reads including methylation statuses at sites of a plurality of cell-free DNA molecules and aligning the sequence reads to a reference genome, wherein the sequence reads are aligned to N sets of one or more CpG sites. The computer system may then, for each set of the N sets of one or more CpG sites, identify a group of sequence reads aligning to the set of one or more CpG sites in the reference genome and determine a methylation level using the methylation statuses of the group of sequence reads. Additionally, the computer system may: generate a feature vector from the N methylation levels; load a machine learning model into memory of the computer system, the machine learning model being trained using training samples having a known chronological age and measured reference vectors of methylation levels; input the feature vector into the machine learning model; and predict, using the machine learning model, the biological age of the subject.

In another embodiment, a method for detecting a pathology in a subject having a known chronological age is provided. The method may be performed by a computer system receiving sequence reads including methylation statuses at sites of a plurality of cell-free DNA molecules and aligning the sequence reads to a reference genome, wherein the sequence reads are aligned to N sets of one or more CpG sites. Then, for each set of the N sets of one or more CpG sites, the computer system may identify a group of sequence reads aligning to the set of one or more CpG sites in the reference genome and determine a methylation level using the methylation statuses of the group of sequence reads. Additionally, the computer system may: generate a feature vector from the N methylation levels; load an age-dependent machine learning model into memory of the computer system, the age-dependent machine learning model being trained using training samples having the known chronological age, known pathology classifications, and measured reference vectors of methylation levels; input the feature vector into the age-dependent machine learning model; and determine, by the age-dependent machine learning model using the feature vector, a classification of a presence of the pathology in the subject.

Additionally, in another embodiment a method for detecting a pathology in a subject having a known chronological age is provided. The method may be performed by a computer system receiving sequence reads including methylation statuses at sites of a plurality of cell-free DNA molecules and aligning the sequence reads to a reference genome. Additionally, for each group of one or more groups of sets of CpG sites, the computer system may: identify a group of sequence reads aligning to any CpG site in the group of sets of CpG sites, the group of sets of CpG sites including at least 3 sets of CpG sites, wherein each set of CpG sites in the group has a same shape classification for a change in a methylation level with respect to age; and determine one or more methylation levels using the methylation statuses of the group of sequence reads. Moreover, the computer system may determine, using a model that varies with age, a classification of a presence of the pathology in the subject, wherein the determining uses the known chronological age of the subject and the one or more methylation levels, and wherein the model is generated using reference samples of subjects having known classifications for the pathology.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows a bar chart of an age distribution of subjects, according to some embodiments of the present disclosure.

FIG. 2 shows a plot of normalized standard deviations for gene-specific 5hmC methylation levels, according to some embodiments of the present disclosure.

FIG. 3 shows a plot of age predictions based on gene-specific 5hmC methylation levels against true chronological ages, according to some embodiments of the present disclosure.

FIG. 4 is a flowchart illustrating a method for measuring a biological age of a subject, according to some embodiments of the present disclosure.

FIG. 5A shows a plot of age predictions based on gene-specific methylation levels against true chronological ages, according to some embodiments of the present disclosure.

FIG. 5B shows a box plot of age acceleration, according to some embodiments of the present disclosure.

FIG. 6 is a flowchart illustrating a method for detecting a disorder in a subject having a known chronological age, according to some embodiments of the present disclosure.

FIG. 7 shows a plot of cfDNA 5hmC trajectories for various genes during ageing, according to some embodiments of the present disclosure.

FIG. 8A shows plots of linear-like gene-specific cfDNA 5hmC trajectories, according to embodiments of the present disclosure.

FIG. 8B shows plots of logarithmic-like gene-specific cfDNA 5hmC trajectories, according to embodiments of the present disclosure.

FIG. 8C shows plots of quadratic-like gene-specific cfDNA 5hmC trajectories, according to embodiments of the present disclosure.

FIG. 8D shows plots of exponential-like gene-specific cfDNA 5hmC trajectories, according to embodiments of the present disclosure.

FIG. 9A shows the plots of gene-specific cfDNA 5hmC trajectories, according to embodiments of the present disclosure.

FIG. 9B shows the plots of gene-specific cfDNA 5hmC trajectories, according to embodiments of the present disclosure.

FIG. 9C shows the plots of gene-specific cfDNA 5hmC trajectories, according to embodiments of the present disclosure.

FIG. 9D shows the plots of gene-specific cfDNA 5hmC trajectories, according to embodiments of the present disclosure.

FIG. 10A shows the plots of gene-specific cfDNA 5hmC trajectories with age indicators, according to embodiments of the present disclosure.

FIG. 10B shows the plots of gene-specific cfDNA 5hmC trajectories with age indicators, according to embodiments of the present disclosure.

FIG. 10C shows the plots of gene-specific cfDNA 5hmC trajectories with age indicators, according to embodiments of the present disclosure.

FIG. 10D shows the plots of gene-specific cfDNA 5hmC trajectories with age indicators, according to embodiments of the present disclosure.

FIG. 11 shows plots of cfDNA 5hmC trajectories during ageing, according to embodiments of the present disclosure.

FIG. 12A shows a plot of organ-specific age prediction against true chronological ages, according to some embodiments of the present disclosure.

FIG. 12B shows a box plot of organ-specific age acceleration, according to some embodiments of the present disclosure.

FIG. 13A shows plots of organ-specific age predictions against true chronological ages, according to some embodiments of the present disclosure.

FIG. 13B shows plots of organ-specific age predictions against true chronological ages, according to some embodiments of the present disclosure.

FIG. 13C shows plots of organ-specific age predictions against true chronological ages, according to some embodiments of the present disclosure.

FIG. 13D shows plots of organ-specific age predictions against true chronological ages, according to some embodiments of the present disclosure.

FIG. 14 shows a plot of Pearson correlation values for organ-specific age models, according to some embodiments of the present disclosure.

FIG. 15 is a flowchart illustrating a method for detecting a disorder in a subject having a known chronological age, according to some embodiments of the present disclosure.

FIG. 16 shows a plot of age predictions based on cytosine methylation levels against true chronological ages, according to some embodiments of the present disclosure.

FIG. 17A shows a plot of Pearson's correlation coefficients between true chronological ages and predicted ages against a number of gene features used in a corresponding age prediction model, according to some embodiments of the present disclosure.

FIG. 17B shows a table of Pearson's correlation coefficients between true chronological ages and predicted ages against a number of gene features used in a corresponding age prediction model, according to some embodiments of the present disclosure.

FIG. 18A shows another plot of Pearson's correlation coefficients between true chronological ages and predicted ages against a number of CpG site features used in a corresponding age prediction model, according to some embodiments of the present disclosure.

FIG. 18B shows another table of Pearson's correlation coefficients between true chronological ages and predicted ages against a number of CpG site features used in a corresponding age prediction model, according to some embodiments of the present disclosure.

FIG. 19A shows a plot of age predictions based on cytosine methylation levels against true chronological ages for controls and HCC patients with different tumor stages of according to some embodiments of the present disclosure.

FIG. 19B shows a box plot of differences in age acceleration for controls and for HCC patients with different tumor stages of according to some embodiments of the present disclosure.

FIG. 20 illustrates a system according to an embodiment of the present invention.

FIG. 21 shows a block diagram of an example computer system usable with system and methods according to certain embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissues from different organisms (mother vs. fetus) or to healthy cells vs. tumor cells.

A “biological sample” refers to any sample that is taken from a subject (e.g., a human, such as a pregnant woman, a person with cancer, or a person suspected of having cancer, an organ transplant recipient or a subject suspected of having a disease process involving an organ (e.g., the heart in myocardial infarction, or the brain in stroke, or the hematopoietic system in anemia)) and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal dialysate, discharge fluid from the nipple, aspiration fluid from different parts of the body (e.g., thyroid, breast), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at, for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

The term “gene” refers to a segment of DNA involved in producing a polypeptide chain or transcribed RNA product. It may include regions preceding and following the coding region (leader and trailer) as well as intervening sequences (introns) between individual coding segments (exons).

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can be at least 1,000, 10,000, 50,000, 100,000, 500,000, 1,000,000, 5,000,000, 10,000,000, 50,000,000, 100,000,000, 500,000,000, one billion, or 3 billion nucleotides long, e.g., a full human genome or a repeat masked human genome. A reference may also include information regarding variations of the reference known to be found in a population of organisms.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

“DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a DNA fragment or whether a particular site in a genome has a particular differential methylation status, e.g., hypermethylation or hypomethylation. A “read” can include information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: e2019768118).

A “methylation level” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules at one or more sites can be determined. The relative abundance can also include an intensity per volume. A methylation level can be determined using a methylation-aware assay such as methylation-aware sequencing or PCR. Example methylation-aware sequencing can include bisulfite sequencing or single molecule techniques, e.g., using nanopores.

A differentially methylated region (DMR) is a genomic region (e.g., set of sites) with different DNA methylation level across two or more biological samples. The different DNA methylation level may be defined by the certain difference in methylation index or density, such as but not limited to 1%, 5%, 10%, 20%, 30%, 40%, 50%, 60%, 70%, 80%, 90%, 95%, 99%, etc. A differentially methylated site (DMS) may be defined in a similar manner.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “shape classification” for methylation can refer to how methylation of a genes changes with age. Different genes will increase or decrease differently, as well as change such an increase or decrease. A given classification can correspond to a particular pattern of change for a gene in one or more methylation levels per age. For example, for a given set of genes, a pattern of change (e.g., a trajectory) of methylation level over ageing time for a gene can be similar. Thus, a set of genes can have a same shape classification. Examples of shape classifications include linear-like, logarithmic-like, quadratic (e.g., convex or concave), and exponential.

The term “biological age” may correspond to characteristics that relate to the actual functional state of an organism, where such characteristics change with time.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The terms “sensitivity” or “true positive rate” (TPR) can refer to the number of true positives divided by the sum of the number of true positives and false negatives. Sensitivity may characterize the ability of an assay or method to correctly identify a proportion of the population that truly has a condition. For example, sensitivity may characterize the ability of a method to correctly identify the number of subjects within a population having an infection. In another example, sensitivity may characterize the ability of a method to correctly identify one or more markers indicative of an infection.

The terms “specificity” or “true negative rate” (TNR) can refer to the number of true negatives divided by the sum of the number of true negatives and false positives. Specificity may characterize the ability of an assay or method to correctly identify a proportion of the population that truly does not have a condition. For example, specificity may characterize the ability of a method to correctly identify the number of subjects within a population not having an infection. In another example, specificity may characterize the ability of a method to correctly identify one or more markers indicative of an infection.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “level of pathology” (also referred to as a condition) can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology.

A “machine learning model” (ML model) can refer to a software module configured to be run on one or more processors to provide a classification or numerical value of a property of one or more samples. An ML model can be generated using sample data (e.g., training samples) to make predictions on test data. Various number of training samples can be used, e.g., at least 10, 100, 1,000, 5,000, 10,000, 50,000 or 100,000 training samples. One example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network, backpropagation, boosting (meta-algorithm), bootstrap aggregating (bagging) such as random forests, support vector machine (SVM), support vector (SVR), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “based on” as used herein means “based at least in part on” and refers to one value (or result) being used in the determination of another value, such as occurs in the relationship of an input of a method and the output of that method. The term “derive” as used herein also refers to the relationship of an input of a method and the output of that method, such

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to +5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Cell-free DNA (cfDNA) can occur naturally in the form of short fragments in various types of biological samples, such as in plasma, urine, saliva, cerebrospinal fluid, pleural fluid, amniotic fluid, peritoneal fluid, and ascitic fluid. In contrast to DNA contained in a particular tissue, plasma or other biological samples can carry cfDNA molecules released from dying cells from various tissue. Thus, examination of cfDNA from biological samples can provide minimally invasive access to DNA molecules from the various tissue. This can enable detection and analysis of abnormal or diseased tissue (e.g., organs). For example, the analysis of cfDNA can be used for noninvasive prenatal testing (Lo et al., 1997), cancer detection (Leon et al., 1977; Mandel, 1948), and organ transplantation monitoring (Lo et al., 1998).

5-Hydroxymethylcytosine (5hmC) and 5-methylcytosine (5mC) are modified forms of cytosine that can affect gene expression. 5hmC levels can be detected from cfDNA using various techniques such as those involving bisulfite sequencing, enzymatic digestion, chemical labeling, antibody-based enrichment, liquid chromatography-tandem mass spectrometry, etc. Cytosine methylation levels (e.g., 5hmC levels, 5mC levels, or a combination thereof) can also be detected from cfDNA using various techniques such as bisulfite sequencing, reduced representation bisulfite sequencing, microarrays, methylated DNA immunoprecipitation, nanopore sequencing, and single molecule real-time (SMRT) sequencing. Changes in 5hmC levels or cytosine methylation levels over time can be estimated based on cfDNA from subjects of different known chronological ages. Such changes in 5hmC levels or cytosine methylation levels can correspond with biological processes such as ageing and disease progression. Thus, 5hmC levels or cytosine methylation levels in cfDNA from a particular subject can be indicative of a state of one or more biological processes (e.g., a biological age or a level of progression for a disease) for the subject.

To determine states of biological processes, approaches to analyze 5hmC levels or cytosine methylation levels for one or more CpG sites can be developed. Additionally or alternatively, approaches to analyze 5hmC levels or cytosine methylation levels for one or more genes can be developed. For example, sequence reads with methylation statuses for one or more sites of one or more cfDNA molecules from a subject can be aligned with a reference genome. The methylation statues may indicate the presence or absence of a 5hmC modification or 5mC modification at each site. As a result of aligning the sequence reads to the reference genome, CpG sites to which the sequence reads correspond can be determined and methylation levels of sets of one or more CpG sites can be derived. In some examples, genes to which the CpG sites correspond can be also identified and gene-specific methylation levels can be derived. For example, for a given set of CpG sites that correspond to a given gene, a corresponding gene-specific methylation level (e.g., a gene-specific 5hmC or cytosine methylation level) can represent a number of CpG sites with a 5hmC or 5mC modification relative to a total number of sites associated with the gene.

Additionally, models can be developed for predicting the states of a biological process. For example, one or more machine learning models can be developed using methylation levels (e.g., 5hmC levels or cytosine methylation levels) for biological samples from subjects of varying age, disease status (e.g., healthy subjects or subjects diagnosed with a particular disease), or a combination thereof. A particular machine learning model may be trained using methylation levels from biological samples of healthy subjects of different ages. As a result of training, the machine learning model may output a predicted biological age based on receiving input with methylation levels for a set of CpG sites, a set of genes, a set of CpG cites that correspond to one or more genes, or a combination thereof. Thus, the machine learning model can utilize time-dependent methylation patterns for the set of CpG sites, the set of genes, the set of CpG cites that correspond to one or more genes, or a combination thereof to predict a biological age of a subject. In some examples, a predicted biological age output by the machine learning model for a subject can be compared to a true chronological age of the subject to reveal age aberrations (e.g., age acceleration or deceleration). Age aberrations can be indicative of a health issue for the subject, such as a presence of a disorder.

In some examples, models can further be developed for classifying a presence or progression of one or more disease. For example, one or more machine learning models can be trained to classify the presence or progression of a disorder based on methylation levels and known chronological ages of subjects. In one example, a machine learning model can be trained to classify the presence of a disorder based on a discrepancy between a known chronological age and a predicted biological age. In another example, machine learning models can be trained on methylation levels for subjects within particular ages ranges and with known pathology classifications (e.g., positive for the disorder or negative for the disorder). The machine learning models may then, based on the training, output the classification of the presence or progression of the disorder based on methylation levels for biological samples from subjects within the age ranges.

Additionally, CpG sites, genes, or CpG sites with corresponding genes that are used for age prediction, disease classification, or a combination thereof can be related with specific tissue types, such as specific organs. For example, specific tissue types may include bone marrow, brain, ovary, pancreas, liver, hypothalamus, heart, kidney, bladder, prostate, lymph nodes, breast, lung, skin, and testis. The tissue type relating to each CpG site and/or gene can be determined based on a CpG site and/or gene being enriched in a biological pathway corresponding to the tissue or based on the CpG site and/or gene being expressed at a high level in the tissue. Methylation patterns for CpG sites and/or genes related to specific tissue types can then be used for tissue- or organ-specific age prediction or disease progression analysis. For example, machine learning models can be trained on methylation levels for CpG sites and/or for genes associated with a particular tissue to predict biological age for the subject. As a result, age acceleration or deceleration of a tissue (e.g., an organ) can be detected. In other examples, the machine learning models can be trained to classify the presence or progression of diseases based on the methylation levels for CpG sites and/or genes associated with a particular tissue.

As a result of obtaining methylation levels (e.g., 5hmC levels or cytosine methylation levels) from cfDNA and developing approaches to predict age, disease occurrence, or disease progression based on the methylation levels, a deeper understanding of related biological processes can be achieved. For example, a deeper understanding of an impact of diseases on particular organs or of effects of aging can be obtained. This can facilitate development of methods for effective detection and treatment of diseases. For example, the organ ageing assessment based on methylation levels in cfDNA can enable disease detection in a minimally invasive manner, which can lead to development of novel preventative interventions.

### I. INTRODUCTION

Biological age can reflect how old an organism is based on physiological or molecular evidence. Biological age can be associated with age-related biological processes and pathophysiological states. For example, if a subject is especially healthy, the subject's biological age may be lower than the subject's chronological age, which can be referred to as ‘decelerated biological ageing’. Otherwise, ‘accelerated biological ageing’ may be detected in subjects with immune-related and/or organ-related dysfunctions and can indicate a high risk of developing one or more illnesses. Hence, the determination of biological age can be important for preventive diagnosis and precision medicine. A standard curve between biological age and physiological or molecular evidence may be constructed from a population of defined normal subjects, so that the biological age can be quantified for each testing sample. The normal subjects can be defined as those who do not have the diseases or disorders being detected during the period of investigation.

Recent advances in molecular biology and omics technologies have enabled the characterization of biological ageing at the molecular level and proposed numerous omics-based ageing clocks to estimate the human biological age (Rutledge et al., 2022). Based on DNA cytosine-phosphate-guanine (CpG) methylation, Hannum et al. predicted chronological age using blood samples (Hannum et al., 2013) and Horvath et al. built the pan-tissue methylation ageing clocks that apply to all human tissues (Horvath, 2013). In addition, blood plasma carries circulating proteins that change during ageing, based on which Lehallier et al. developed an accurate model predictive of chronological age (Lehallier et al., 2019) and Oh et al. recently demonstrated organ-specific proteomic ageing clocks in living individuals (Oh et al., 2023). The transcriptomic clock is another type, and ageing clocks can be derived using transcriptomic data from different tissues such as the peripheral blood (Peters et al., 2015) and dermal fibroblasts (Fleischer et al., 2018). Moreover, analyzing metabolites in the urine (Hertel et al., 2016) and blood (Robinson et al., 2020) has generated metabolomic ageing clocks.

5-methylcytosine (5mC) is a predominant methylated form of DNA, and the TET enzymes gradually oxidize 5mC to a series of intermediate states such as 5-hydroxymethylcytosine (5hmC) during active demethylation (Greenberg and Bourc'his, 2019). Studies have shown that 5hmC not only acts as an intermediate in the demethylation process but also as a stable epigenetic mark with independent regulatory functions (Lister et al., 2013; Pastor et al., 2011). Neurodevelopment might be associated with the enrichment of 5hmC in the brain and neuronal cells (Kriaucionis and Heintz, 2009; Szulwach et al., 2011), and 5hmC accumulation appears to be essential for preserving the pluripotency of embryonic stem cells (Koh et al., 2011). A recent murine model study revealed that 5hmC accumulates in multiple aged tissues including the liver, heart, and lung, in contrast to 5mC which does not show any detectable difference between young and aged organs (Occean et al., 2023). Besides, Xiong et al. demonstrated a negative correlation (r=−0.865) between chronological age and the 5hmC level of human blood cells, while blood 5mC showed a much weaker correlation with age (r=−0.232) (Xiong et al., 2015).

However, the analysis of blood cells could not reflect the ageing of other organs. The plasma DNA could be derived from different organs, in theory offering an opportunity to assess organ-specific ageing. However, there are no established approaches for this purpose based on the analysis of plasma DNA.

### II. VARIABILITY OF METHYLATION IN GENES ACROSS AGE COHORT

Data for subjects of varying ages can be obtained and can include for, for each of a set of genes of each subject, a gene-specific methylation level. The gene-specific methylation level can be a 5hmC level, which can indicate a concentration or amount of 5hmC modifications present in each gene for each subject. By comparing the gene-specific methylation levels across subjects of varying ages, highly variable genes can be identified. The highly variable genes can be the genes that exhibit the most variation in 5hmC level across the subjects of varying ages.

### A. Cohort

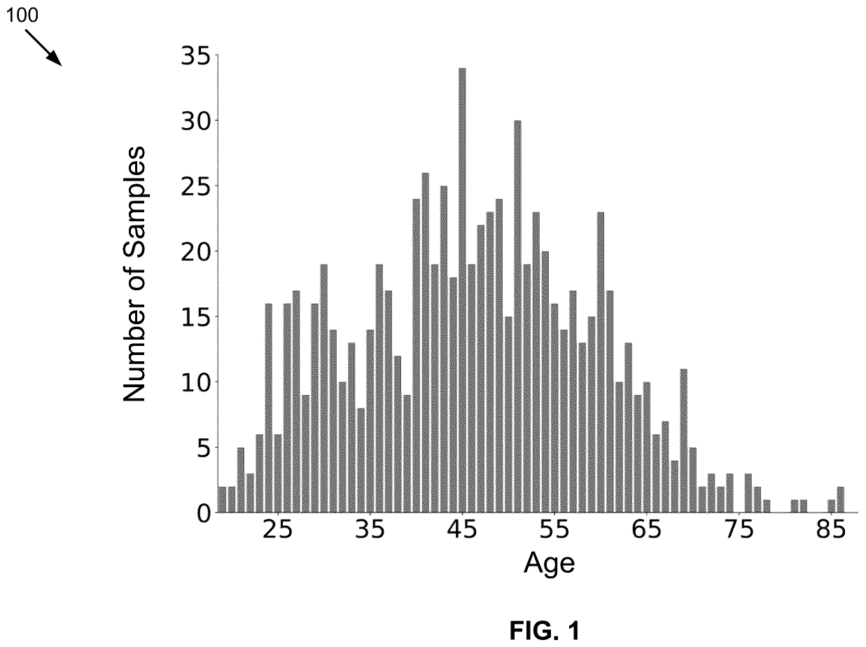

Data from a cohort of 785 subjects can be used in some embodiments of the present disclosure to determine genes with variable cfDNA 5hmC signals over time. The data can be derived from two published genome-wide 5hmC datasets. The first dataset can include data for subjects without cancer, including 522 healthy subjects and 263 subjects with chronic hepatitis B virus infection (HBV) or cirrhosis. An age range of the subjects in the first dataset can be between nineteen and eighty-six. FIG. 1 shows a bar chart 100 of an age distribution of the subjects in the first dataset. For example, each bar in the bar chart 100 can how many subjects of each age between nineteen and eighty-six are included in the first dataset. The ages in the bar chart 100 can be the true chronological ages (“true ages”) of the subjects. The second dataset can include data for 47 subjects with cancer. The subjects with cancer can have a glioma. An age range for the second dataset can span from twenty-three to sixty-six years old.

For each subject, there can be data for 17,000 genes. The data for each gene can include the gene-specific methylation level (e.g., the concentration or amount of 5hmC modifications present in each gene for each subject). In an example, a 5hmC-Seal technique can be performed on one or more plasma cfDNA samples from each subject to determine a 5hmC level for each gene. Thus, for each subject, the first and second datasets can include the genes and corresponding 5hmC levels.

### B. Identification of Highly Variable Genes

The highly variable genes can be the genes that exhibit the most variation in 5hmC level across the cohort. In some examples, the variability in cfDNA 5hmC signals for each gene can be measured by computing a standard deviation for the 5hmC levels for each gene across the cohort. Alternatively, the variability in cfDNA 5hmC signals for each gene can be measured by computing a normalized standard deviation for the 5hmC levels for each gene across the cohort. The normalized standard deviation can be obtained by scaling the standard deviation for the 5hmC levels for each gene with a mean 5hmC level for genes falling into a given bin (Satija et al., 2015). For example, the genes can be grouped into bins based on mean 5hmC levels. For each bin, there can be a corresponding mean 5hmC level. The standard deviation for each gene can therefore be normalized with the mean 5hmC level for its corresponding bin.

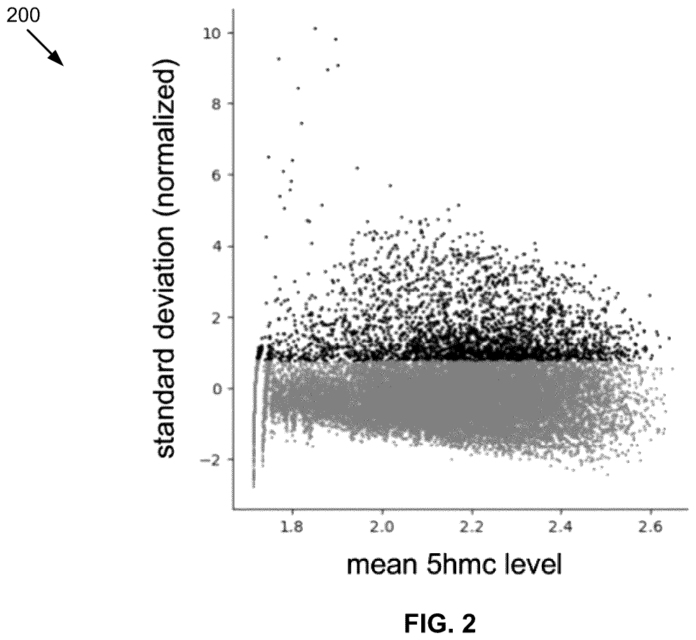

FIG. 2 shows a plot 200 of normalized standard deviations for gene-specific methylation levels, according to some embodiments of the present disclosure. More specifically, FIG. 2 shows the normalized standard deviations for the 5hmC levels for each gene across the cohort. In FIG. 2, the normalized standard deviation for each gene can be plotted against a corresponding mean 5hmC level for the gene. Genes with a highest variability in 5hmC levels can be the genes with highest normalized standard deviation values for the 5hmC levels. For example, in FIG. 2 a top 3,000 out of the 17,000 genes with highest normalized standard deviation values are shown with solid black dots.

In some examples, prior to computing a mean and standard deviation for the 5hmC levels for a gene, a logarithmic transformation can be performed on each 5hmC level. The mean 5hmC level for a gene can then be computed using the log-transformed 5hmC values for the gene for each of the subjects in the cohort. Additionally, the standard deviation for the 5hmC levels for a gene can be computed using the log-transformed 5hmC values for the gene. The standard deviations can further be normalized with mean 5hmC levels corresponding to one or more bins. Then, based on the normalized standard deviations for each gene, the top variable genes in terms of the 5hmC level in cfDNA can be identified. The top variable genes can be atop 10, top 50, top 100, top 500, top 1000, top 2000, top 3000, top 4000, top 5000, etc., or atop 1%, top 2%, top 5%, top 10%, top 20%, top 30%, top 40%, top 50%, etc.

In addition or alternative too standard deviation, metrics for statistical dispersion can include interquartile range (IQR), range, median absolute deviation, mean absolute difference, root-mean-square deviation (RMSD), distance correlation, or the like. Any one or more of the metrics for statistical dispersion can be used to identify genes with high 5hmC level variability.

### III. RELATIONSHIP BETWEEN BIOLOGICAL AGE AND METHYLATION LEVEL

A biological age of a subject or of a particular tissue can be estimated based on methylation levels of one or more sets of sites (e.g., CpG sites), which can be partially or entirely within respective genes. As examples, the methylation levels can be of 5mC and/or 5hmC. Thus, in one example, the 5hmC levels can be of one or more genes. To correlate age and such methylation levels of sets of sites (e.g., 5hmC levels of genes), methylation level (e.g., 5hmC level) trajectories can be derived. The methylation level trajectories can show typical methylation levels over time for one or more genes based on cfDNA samples from subjects without cancer.

### A. CfDNA Methylation-Based Ageing Clock

The methylation level (e.g., 5hmC and/or 5mC) trajectories can be derived using various algorithms such as absolute shrinkage and selection operator (LASSO), ridge regression, support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm.

A model for deriving methylation level trajectories, for predicting age based on the methylation level trajectories, or a combination thereof may utilize linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, etc. A model trained using cfDNA methylation levels for one or more sites (e.g., CpG sites) to predict biological age can be referred to herein as an ageing clock.

1. 5hmC-Ageing Clock

In a particular example, a regression model (e.g., a LASSO regression model) can be developed for predicting biological age based on 5hmC values for one or more genes. The model may use data for at least a portion of the genes with highly variable 5hmC levels as identified above based on the datasets for the cohort. For example, the regression model can be trained and tested using 5hmC levels for the top 3,000 genes corresponding to the block dots depicted in FIG. 2.

To train and verify the regression model, the data (e.g., the 3,000 genes and corresponding 5hmC levels) for each subject without cancer in the cohort can be split into a training dataset and a testing dataset. In a particular example, the data can be split into the training dataset and the testing dataset with a ratio of 9:1. The training can include fitting the regression model to the training dataset. That is, training can include tuning hyperparameters associated with the regression model to improve age prediction by the regression model based on the 5hmC levels for the highly variable genes. In some examples, during training, the regression model may select a minimum set of genes that may be sufficient for predicting biological age. For example, the regression model may select 200 of the 3000 highly variable genes as the minimum set of genes usable to predict biological age. The selected 200 genes are listed in table 3 below.

After training, the regression model can be tested by inputting 5hmC levels for at least the selected 200 genes into the trained regression model. The 5hmC levels input into the regression model can be for the subjects without cancer in the testing dataset. The trained regression model can then output predicted ages for the subjects in the testing dataset based on the 5hmC levels for the selected genes. The predicted ages can be compared to true ages of the subjects to estimate an accuracy of the trained regression model.

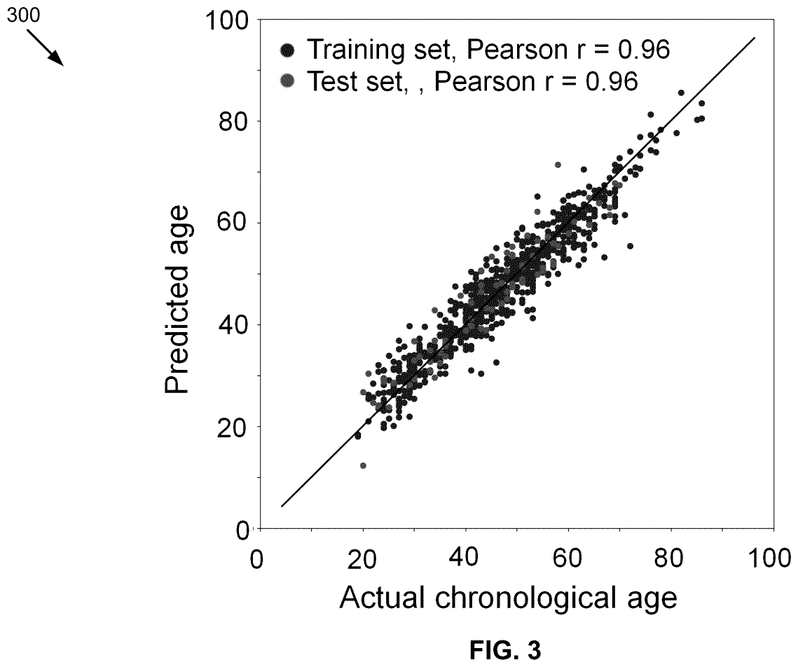

FIG. 3 shows a plot 300 of ages predicted based on gene-specific methylation levels against true chronological ages, according to some embodiments of the present disclosure. More specifically, the plot 300 depicts the predicted ages output by the trained regression model for the training dataset and the predicted ages output by the trained regression model for the testing dataset. Thus, FIG. 3 includes a predicted age for each subject without cancer in the cohort. The predicted ages can be plotted against true ages of each subject in the cohort, which may be included in the first dataset. As shown in FIG. 3, the ages predicted by the trained regression model can correlate with the true ages in the first dataset. To quantify the correlation between the predicted and true ages depicted in FIG. 3, Pearson's correlation coefficient can be computed. The Pearson's correlation coefficient for the predicted and true ages depicted in FIG. 3 is 0.96.

### 2. Ageing Clock Based on Cytosine Methylation Levels

Age prediction can be performed based on 5hmC and 5mC levels. Cytosine methylation may encompass both 5-methylcytosine (5mC) and 5-hydroxymethylcytosine (5hmC). Bisulfite sequencing may be used for detecting methylated cytosines of both types. In an example, during bisulfite treatment, unmethylated cytosines are converted to uracils, which are then read as thymidines during sequencing. In contrast, during bisulfite treatment, methylated cytosines, including both 5mC and 5hmC, remain unchanged and are read as cytosines. Thus, in bisulfite sequencing, a cytosine methylation level (e.g., a signal indicative of a 5mC level and a 5hmC level of a CpG site) can be determined by the ratio of the reads identified as cytosines. Cytosine methylation patterns may be obtained via other techniques, such as reduced representation bisulfite sequencing, microarrays, methylated DNA immunoprecipitation, nanopore sequencing, and single molecule real-time (SMRT) sequencing, in other examples.

A biological age of a subject or of a particular tissue can be estimated based on the cytosine methylation of one or more CpG sites (CpGs) and/or genes. To correlate age and cytosine methylation level, cytosine methylation level trajectories can be derived. The cytosine methylation level trajectories can show typical cytosine methylation levels over time for one or more CpGs based on cfDNA samples from subjects without cancer.

Additionally, aging analysis can be performed for diseased subjects. The diseased subjects may exhibit abnormal results during age analysis. For example, accelerated or decelerated aging can be indicative of a particular disorder. Thus, by predicting age based on cytosine methylation levels and comparing a predicted age to a true age of a subject, disorders or other pathologies can be detected.

Cytosine methylation level trajectories can be derived using various model techniques (as described above) such as least absolute shrinkage and selection operator (LASSO), ridge regression, support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm.

Further examples of models for deriving cytosine methylation level trajectories, for predicting age based on the cytosine methylation level trajectories, or a combination thereof include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, etc. A model trained using cytosine methylation levels for one or more CpGs and/or genes to predict biological age can be referred to herein as an ageing clock (or ageing clock model).

In a particular example, a regression model (e.g., a LASSO regression model) can be developed for predicting biological age based on cytosine methylation levels for one or more CpGs. A LI-regularization term may be incorporated into the regression model to prevent overfitting. To train and verify the regression model, data indicative of cytosine methylation levels for subjects without cancer can be obtained.

In the particular example, whole-genome bisulfite sequencing of plasma cfDNA samples from 136 subjects (median: ˜193 million paired-end reads) without cancer may be performed to obtain data that includes cytosine methylation levels for CpGs from the subjects. The data can then be split into a training dataset and a testing dataset. In the particular example, the 136 subjects without cancer are split into a training dataset and a testing dataset with a 4:1 ratio. As such, the training data set includes data indicative of cytosine methylation levels for a training subset of the subjects and the testing dataset includes data indicative of cytosine methylation levels for a testing subset of the subjects.

The training of the regression model can include fitting the regression model to the training dataset. That is, training can include tuning hyperparameters associated with the regression model to improve age prediction by the regression model based on the cytosine methylation levels of the training subset of subjects. In some examples, during training, the regression model may select genes and/or CpGs whose methylation levels are predictive of age. Additionally, in the particular example, the regression model selected 49 CpGs, which are listed in table 4 below.

After training, the regression model can be tested by inputting cytosine methylation levels for the testing subset of subjects into the trained regression model. The trained regression model can then output predicted ages for the testing subset of subjects based on the cytosine methylation levels for the selected CpGs. The predicted ages can be compared to true chronological ages of the subjects to estimate an accuracy of the trained regression model.

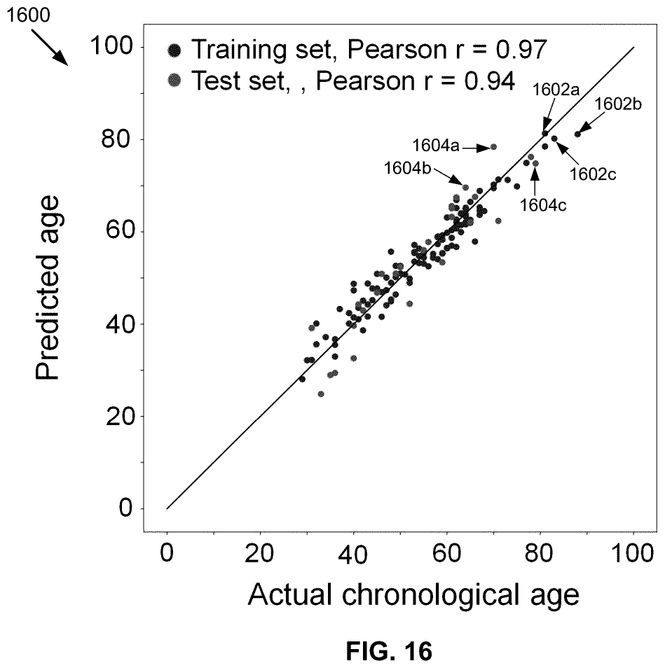

FIG. 16 shows a plot 1600 of ages predicted based on cytosine methylation levels against true chronological ages, according to some embodiments of the present disclosure. More specifically, the plot 1600 depicts the predicted ages output by the trained regression model for the training dataset against true chronological ages for the training set (e.g., as shown by points 1602a-c on plot 1600). Plot 1600 also depicts the predicted ages output by the trained regression model against true chronological ages for the testing set (e.g., as shown by points 1604a-c on plot 1600). Thus, FIG. 16 includes a predicted age for each of the 136 subjects without cancer.

As shown in FIG. 16, the ages predicted by the trained regression model correlate with the true chronological ages. To quantify the correlation between the predicted and true chronological ages depicted in FIG. 16, Pearson's correlation coefficient can be computed. The Pearson's correlation coefficient for the training dataset was 0.97 and the Pearson's correlation coefficient for the testing dataset was 0.94. Thus, plot 1600 shows that the ages predicted by the trained regression model using cfDNA cytosine methylation levels closely correlate with the actual chronological ages in both the training and testing datasets. B. Example method for age prediction

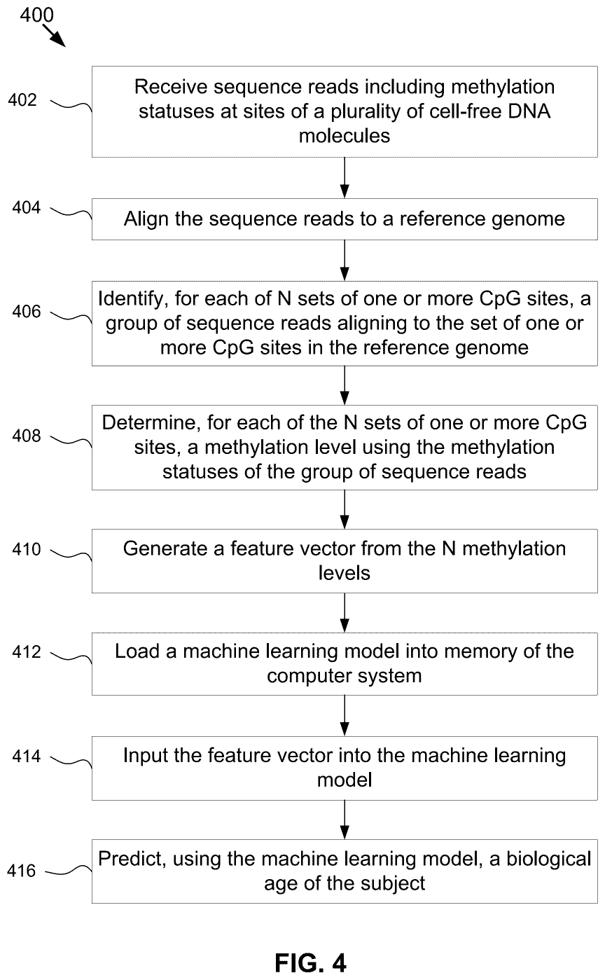

FIG. 4 is a flowchart illustrating a method 400 for measuring a biological age of a subject, according to some embodiments of the present disclosure.

At block 402, the method 400 can include receiving sequence reads including methylation statuses at sites of a plurality of cell-free DNA (cfDNA) molecules. The cfDNA molecules can be obtained from plasma or another suitable biological sample from a subject. The sequence reads can be determined using sequencing or probe-based techniques, as may be done using a microarray or in an amplification reaction (e.g., PCR), performed on the biological sample from the subject. The sequencing can be paired-end sequencing, single-molecule sequencing, targeted sequencing, or the like. The sequence reads can include one or more sites for each of the cfDNA molecules, where the sites can be specific positions or loci in the cfDNA molecule. In some examples, the sites can be of a particular type of interest. For example, the sites of interest can include CpG sites or other suitable sites, e.g., an A. In such examples, there can be sequence reads received that do not include the sites of interest (e.g., the CpG sites). The sequence reads that do not include the sites of interest may be discarded and not used in the method 400.

The methylation statuses can indicate whether each site is chemically modified by the addition of a CH3 group to form a 5mC or 5mhC group, or other type of methylation. Determining the methylation status of each site can, in some examples, include (1) treating the plurality of cell-free DNA molecules (e.g., with bisulfite or a restriction enzyme) or (2) analyzing optical or electrical signals of the plurality of cell-free DNA molecules at positions within a window that includes the site. Further details can be found in U.S. Publication No. 2021/0047679 and 2022/0328135.

At block 404, the method 400 can include aligning the sequence reads to a reference genome. The reference genome can be a representative example of the DNA sequence of a species corresponding to the subject. The sequence reads may be aligned to the reference genome by searching for a location in the reference genome that best matches the sequence read.

The sequence reads may be aligned to N sets of one or more CpG sites in the reference genome. As examples, N is an integer that can be at least 3, 4, 5, 6, 7, 8, 9, 10, 11, 12, 13, 14, 15, 16, 17, 18, 19, 20, 30, 40, 50, 60, 70, 80, 90, or 100. Thus, there can be at least any of these numbers of CpG sites. Examples of CpG sites to which sequence reads may be aligned are provided in Table 4. Additionally or alternatively, the sequence reads may be aligned with genes in the reference genome. The genes may include any number of the 3,000 genes, e.g., corresponding to the dots depicted in FIG. 2 and/or provided in Table 1. In some examples, at least some of the sets of one or more CpG sites may correspond to genes. Thus, at least some of the sequence reads may be aligned with sets of one or more CpG sites within a gene. Table 4 shows examples of CpG sites that correspond with genes. The skilled person will appreciate that any suitable alignment tool can be used, e.g., Bowtie, BWA, BLAST, and FASTA.

At block 406, the method 400 can include, for each of the N sets of one or more CpG sites, identifying a group of sequence reads aligning to the set of one or more CpG sites in the reference genome. For example, the reference genome can include annotations or other suitable indications of locations and boundaries of CpG sites, genes, or a combination thereof. Therefore, based on aligning the sequence reads with the reference genome, each sequence read can be associated with a CpG site, gene, or a combination thereof. Each sequence read may correspond with one or more CpG sites, genes, or a combination thereof. Each of the CpG sites, genes, or the combination thereof may be associated with a particular tissue type, such as a particular organ. In one example, the particular tissue type can be selected from a group comprising bone marrow, brain, ovary, pancreas, liver, hypothalamus, heart, kidney, bladder, prostate, lymph nodes, breast, lung, skin, and testis.

The association of each of the CpG sites, genes, or the combination thereof with the particular tissue type can be determined based on (1) a biological pathway, (2) epigenetic patterns, or (3) expression levels in the particular tissue type being greater than a threshold. With respect to a biological pathway, the particular tissue type can be determined based on the CpG sites, genes, or the combination thereof participating in a biological pathway related the particular tissue. The biological pathway can be a sequence of molecular events within a biological environment (e.g., a cell), which may be carried output by gene products (e.g., proteins). For example, the INS gene, which encodes insulin, can be associated with the pancreas based on insulin being used in the biological pathway of glucose homeostasis. With respect to expression levels, some CpG sites or genes can be expressed at high levels (e.g., greater than the threshold) in particular tissues. Thus, for a given gene, if a corresponding expression level in a particular tissue (e.g., heart) is greater than a threshold, the gene can be associated with the particular tissue.

At block 408, the method 400 can include, for each of the N sets of one or more CpG sites, determining a methylation level using the methylation statuses of the group of sequence reads. In some examples, the methylation level can include a count (number) of methylated sites (or unmethylated sites) aggregated over the sequence reads for a given CpG site or gene. For example, to determine a methylation level at the set of one or more CpG sites, an amount (number) of the methylation statuses at the one or more CpG sites that indicate a methylation is present or that indicate the methylation is not present may be determined. Thus, in some examples, a methylation level of a group of sequence reads aligning to a set of one or more CpG sites can be an amount of methylation statuses for each of the CpG sites in the set that indicate a methylation is present.

Alternatively, in examples in which the sequence reads are aligned with genes in the reference genome, the methylation level may be a count (number) of methylated sites (or unmethylated sites) aggregated over the sequence reads for each gene. Thus, the methylation levels may be gene-specific methylation levels. Additionally, in examples in which at least some of the sets of one or more CpG sites correspond to genes, the methylation levels may be gene-specific methylation levels. For example, the methylation level may be an amount of methylation statuses for each CpG site corresponding to gene that indicate a methylation is present.

The number of methylated sites can be normalized. For instance, the methylation level can be a proportion, percentage, or other suitable quantification of a methylation density for the set of one or more CpG sites or for a gene. That is, the methylation levels can be a measure of a number of methylated sites in the sequence reads associated with the set of one or more CpG sites or the gene relative to a total number of sites in the sequence reads associated with the set of one or more CpG sites or the gene.

At block 410, the method 400 can include generating a feature vector from the N methylation levels. The feature vector can include the methylation level for each set of one or more CpG sites or gene. The feature vector can include the methylation levels in a structured form that can be ingested (input) into and understood by a machine learning model.

At block 412, the process can include loading a machine learning model into memory of the computer system. The machine learning model can be trained using training samples having a known chronological age (i.e., taken from a training subject with the known chronological age) and measured reference vectors of methylation levels measured from the training samples, i.e., from biological samples of training subject). The machine learning model can be a regression model or another suitable type of machine learning model. In an example, the training samples can be obtained from a training cohort, e.g. like cohorts described herein. The training samples can be subjects without cancer. The training cohort can include a known chronological age for each subject without cancer. Additionally, the reference vectors of methylation levels can include the 5hmC levels for at least a portion of the highly variable genes.

At block 414, the process can include inputting the feature vector into the machine learning model. For example, the feature vector can be input into a regression model. The feature vector (and other feature vectors described herein) can have various formats, e.g., organized serially as a single array or organized into a multi-dimensional as may be done when other values besides methylation levels are also included in the feature vector.

At block 416, the process can include predicting, using the machine learning model, a biological age of the subject. As examples, the biological age predicted can be a year (e.g., 20, 30, 45, 55, etc.) or the biological age can be an age range (e.g., 20-25, 30-39, etc.), or even higher resolution than a year, e.g., a month of range of months. In examples in which the genes are associated with a particular tissue type, the biological age can be for the particular tissue type. Additionally, in some examples, the biological age predicted using the machine learning model can be compared to a true chronological age of the subject. If the predicted age deviates from the true chronological age, e.g. greater than the true chronological age by a threshold amount, the subject can be determined to have a pathology (e.g., a condition, disease or disorder). In such an instance, an alert or other suitable indicator of age acceleration can be generated and output. In the examples, in which the sets of one or more CpG sites or genes are associated with a particular tissue type, the predicted age being greater than the chronological age by at least the threshold amount may be indicative of a presence of a pathology that affects the particular tissue type.

### IV. PATHOLOGY DETECTION

Aging analysis can be performed for diseased subjects. The diseased subjects may exhibit abnormal results during age analysis. For example, accelerated aging for a particular tissue can be indicative of a particular disorder. Thus, by predicting age based on cfDNA methylation levels (e.g., 5hmC levels and/or 5mC levels) and comparing the predicted age to a true age of the subject, disorders or other pathologies can be detected. Additionally or alternatively, by analyzing methylation levels for sets of CpG sites and/or genes with highly variable methylation levels over time and comparing the methylation levels to reference methylation levels of a known age, pathologies can be detected. The comparison can occur via a machine learning model that is trained using the reference methylation levels (reference vectors).

### A. Example Model Based on 5hmC Levels

To investigate the biological ageing in diseased subjects based on cfDNA methylation levels signals, the second dataset can be input into the regression model. As described above, based on the data for the subjects without cancer in the cohort, the regression model selected 200 genes that can be used to predict biological age. Of the 200 genes, 175 may have detectable 5hmC levels in the second dataset. Thus, to accommodate for the differences in the datasets, the regression model can be retrained. For example, data for the subjects without cancer in the first dataset can be used to retrain the model. The data for the subjects without cancer used to retain the model can include the 175 genes and corresponding 5hmC levels. Similar to the previous example, the data can be split into a training dataset and a testing dataset with a ratio of 9:1.

After training, the 5hmC levels from the testing dataset can be input into the regression model. Additionally, the 5hmC levels from the second dataset can be input into the regression model.

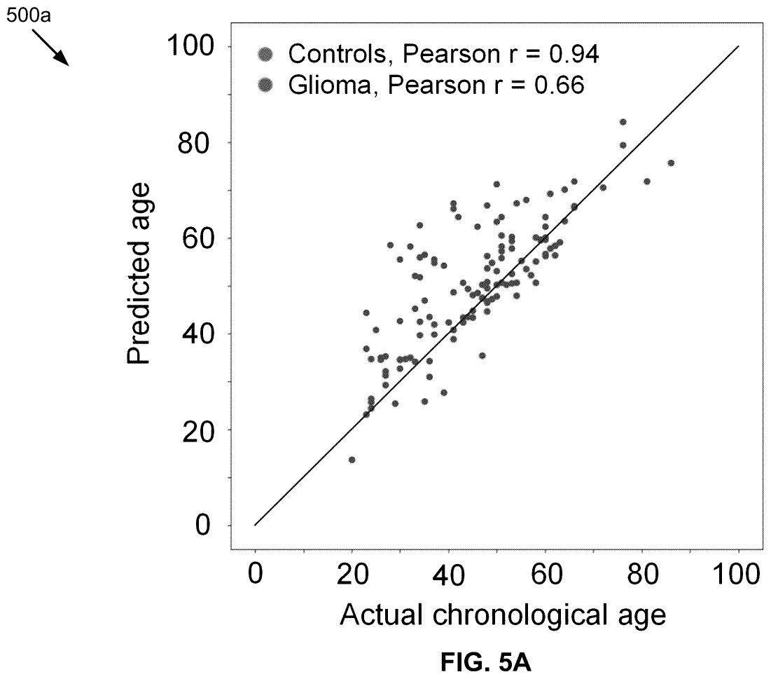

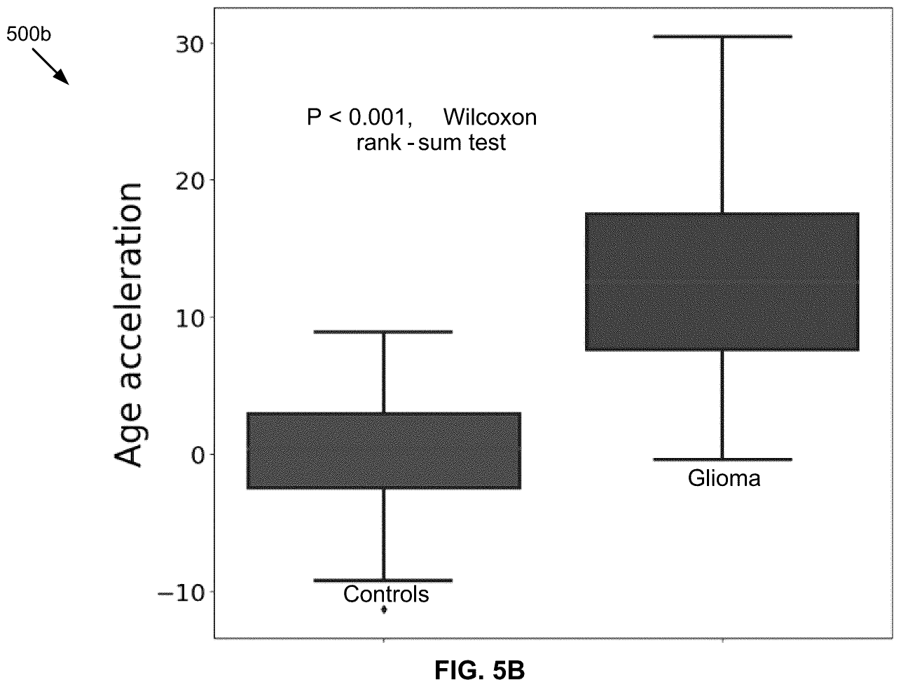

FIG. 5A shows a plot 500a of age predictions based on gene-specific methylation levels against true chronical ages, according to some embodiments of the present disclosure. In particular, the plot 500a can include predicted ages output by the model for the testing dataset and for the second dataset. As depicted in FIG. 5A, the regression model can accurately predict the true chronological ages for the subjects without cancer in the testing dataset. For example, the Pearson's correlation coefficient for the predicted vs true ages for the testing dataset is 0.94. Further, as shown in FIG. 5A, the predicted ages for the subjects in the second dataset can moderately correlate with the true ages of the subjects. For example, the Pearson's coefficient for the predicted vs true ages for the second dataset is 0.66. Additionally, as depicted, the predicted ages corresponding to the second dataset can generally be greater than the true ages of the subjects.

Based on the predicted ages output by the regression model for the second dataset generally being greater than the true ages, it can be predicted that subjects with glioma may exhibit age acceleration. This prediction can be based on gene-specific methylation levels (e.g., the 5hmC levels) of the subjects in the second dataset in comparison with control subjects (e.g., the subjects without cancer in the first dataset). Age acceleration can be defined as a difference in years between a predicted age and true age. To determine whether the subjects with glioma exhibit age acceleration, a determination can be made as to whether differences in age acceleration between control subjects and the subjects with glioma are significant. To do so, a Wilcoxon rank-sum test can be performed.

FIG. 5B shows a box plot 500b of age acceleration, according to some embodiments of the present disclosure. In particular, the box plot 500b can depict results of the Wilcoxon rank-sum test. For example, as depicted in the box plot 500b, a median age acceleration for the glioma subjects can be around thirteen years, while a median age acceleration for the control subjects can be around one year. The p-value for the Wilcoxon rank-sum test can be below 0.001, suggesting that there is a significant difference between the predicted ages for the control subjects and the subjects with glioma. Based on these results, it can be determined that the glioma patients likely do exhibit age acceleration. Previous studies (Liao et al., 2018; Lin and Wagner, 2015) can also show that glioma tumour tissues exhibit age acceleration compared with healthy tissue based on Horvath's methylation ageing clock.

Thus, predicting age based on gene-specific methylation levels can be useful for identifying abnormally accelerated or decelerated biological age. This can enable effective detection, prevention, and treatment of diseases.

### B. Detecting Pathology Using Combined 5hmc and 5mC

To investigate biological ageing in diseased subjects based on cytosine methylation levels (e.g., based on 5hmC levels and 5mC levels), data that includes methylation levels obtained via whole-genome bisulfite sequencing of plasma cfDNA samples from 38 subjects with early-stage (n=20), intermediate-stage (n=11), and advanced-stage (n=7) cancer can be input into the regression model described above with respect to FIG. 16. The regression model may then output the predicted ages for the subjects with early-stage, intermediate-stage, and advanced-stage cancer.

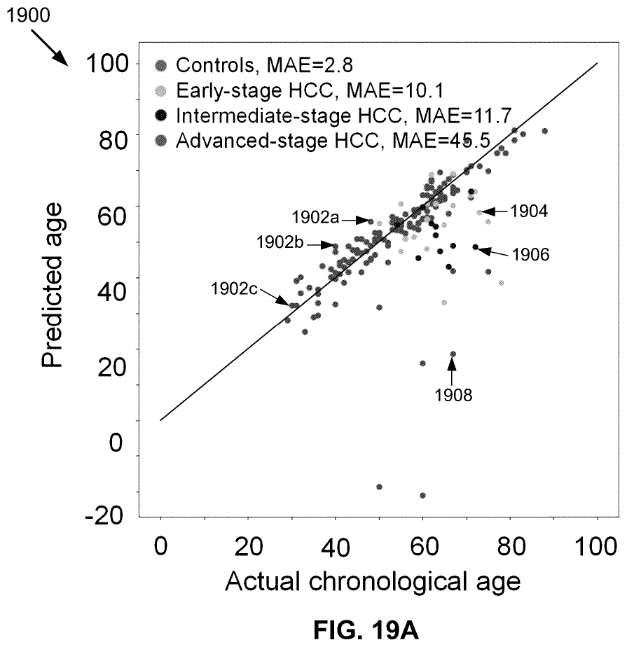

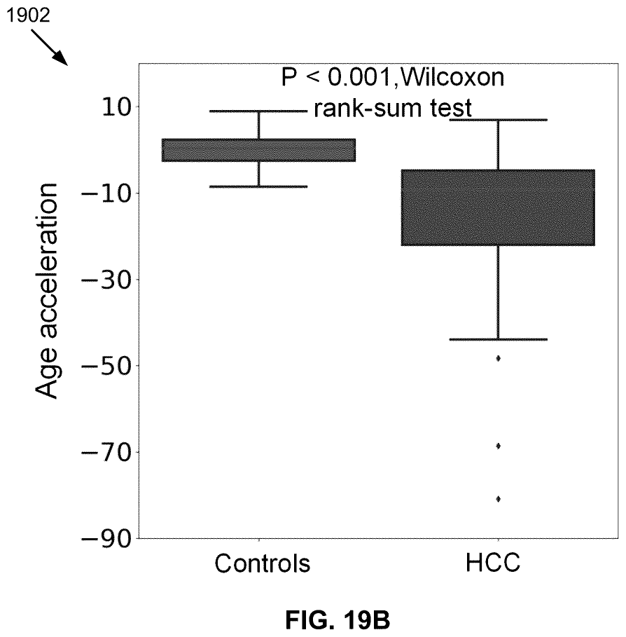

FIG. 19A shows a plot 1900 of age predictions based on cytosine methylation levels against true chronological ages for controls and different stages of Hepatocellular Carcinoma (HCC) according to some embodiments of the present disclosure. Plot 1900 depicts age predictions for subjects without cancer (referred to as controls in plot 1900) plotted against true chronological ages for the subjects (e.g., as shown by points 1902a-c). Plot 1900 also depicts age predictions for subjects with subjects with early-stage cancer (e.g., as shown by point 1904), intermediate-stage cancer (e.g., as shown by point 1906), and advanced-stage cancer (e.g., as shown by point 1908).

Accordingly, plot 1900 shows differences between age predictions and true chronological ages for the subjects without cancer and for the subjects with early-stage, intermediate-stage, and advanced-stage cancer. Compared to the subjects without cancer, subjects with early-stage, intermediate-stage, and advanced-stage cancer have larger error (e.g., mean absolute error (MAE)) between their true chronological and predicted ages. In particular, the MAE for the subjects without cancer was 2.8 while the MAE for subjects with early-stage cancer was 10.1, the MAE for subjects with intermediate-stage cancer was 11.7, and the MAE for subjects with advanced-stage cancer was 45.5.

FIG. 19B shows a box plot 1902 of age acceleration of the subjects without cancer and for the subjects with HCC, according to some embodiments of the present disclosure. In particular, the box plot 1902 depicts results of a Wilcoxon rank-sum test. For example, as depicted in the box plot 1902, a median age acceleration for the subjects with HCC can be around negative 10 years. The p-value for the Wilcoxon rank-sum test can be below 0.001, suggesting that there is a significant difference between the predicted ages for the subjects without cancer and the subjects with HCC. Based on these results (e.g., based on the age acceleration being negative and there being a significant difference), it can be determined that the subjects with HCC likely exhibit age deceleration relative to subjects without cancer.

Thus, predicting age based on methylation levels (e.g., based on 5mC and 5hmC levels) can be useful for identifying abnormally accelerated or decelerated biological age. This can enable effective detection, prevention, and treatment of diseases.

### C. Other Models

In another example, to investigate the biological ageing in diseased subjects based on cfDNA 5hmC signals, a machine learning model can be trained on data from the second dataset corresponding to subset with cancer and data from the first data set corresponding to subjects without cancer. The data can be split into a training dataset and a testing dataset. The data can include a pathology classification (e.g., positive for glioma or negative for glioma) and 5hmC levels of highly variable genes. The machine learning model may use logistic regression, one or more decision trees, a neural network, or the like. During training, the machine learning model can learn to predict a classification of a presence of the disorder (e.g., whether or not a subject has a glioma) based on 5hmC levels of a set of genes. After training, the 5hmC levels from the testing dataset can be input into the machine learning model. The machine learning model can then output a predicted classification of a presence of glioma for each subject of the testing dataset.

In yet another example, to investigate the biological ageing in diseased subjects based on cfDNA 5hmC signals, one or more machine learning models can be trained on data from the second dataset for subjects within particular age ranges and data from the first data set for subjects in the particular age ranges. For each age range, the corresponding data can be split into a training dataset and a testing dataset. The data can include ages of each subject, a pathology classification (e.g., positive for glioma or negative for glioma), and 5hmC levels of highly variable genes. The machine learning models may include logistic regression, a decision tree, a neural network, or the like. During training, the machine learning models can each learn to predict a classification of a presence of the disorder or other pathology (e.g., whether or not a subject has glioma) based on 5hmC levels of a set of genes and based on an age of the subject. After training, similar to the above, the machine learning model can output a predicted classification of a presence of glioma for each subject of the testing dataset.

In another example, to investigate the biological ageing in diseased subjects based on cfDNA methylation levels, a machine learning model can be trained on the data that includes methylation levels obtained via whole-genome bisulfite sequencing of plasma cfDNA samples from the 38 subjects with early-stage (n=20), intermediate-stage (n=11), and advanced-stage (n=7) cancer and from the data obtained via whole-genome bisulfite sequencing of plasma cfDNA samples from the 136 subjects without cancer. The data can be split into a training dataset and a testing dataset. The data can include a pathology classification (e.g., positive for HCC or negative for HCC) and methylation levels of sets of CpG sites (e.g., the CpG sites provided in Table 4). The machine learning model may use logistic regression, one or more decision trees, a neural network, or the like. During training, the machine learning model can learn to predict a classification of a presence of the disorder (e.g., whether or not a subject has HCC) based on methylation levels of one or more sets of CpG sites. After training, the methylation levels from the testing dataset can be input into the machine learning model. The machine learning model can then output a predicted classification of a presence of cancer (e.g., HCC) for each subject of the testing dataset.

In yet another example, to investigate the biological ageing in diseased subjects based on cfDNA methylation levels, one or more machine learning models can be trained on the data that includes methylation levels obtained via whole-genome bisulfite sequencing of plasma cfDNA samples from the 38 subjects with early-stage (n=20), intermediate-stage (n=11), and advanced-stage (n=7) cancer and from the data obtained via whole-genome bisulfite sequencing of plasma cfDNA samples from the 136 subjects without cancer. For each of multiple age ranges of the subjects, the corresponding data can be split into a training dataset and a testing dataset. The data can include ages of each subject, a pathology classification (e.g., positive for HCC or negative for HCC), and methylation levels of sets of CpG sites. The machine learning models may include logistic regression, a decision tree, a neural network, or the like. During training, the machine learning models can each learn to predict a classification of a presence of the disorder or other pathology (e.g., whether or not a subject has glioma) based on methylation levels of one or more sets of CpG sites and based on an age of the subject. After training, similar to the above, the machine learning model can output a predicted classification of a presence of cancer (e.g., HCC) for each subject of the testing dataset.

### D. Method for Pathology Prediction

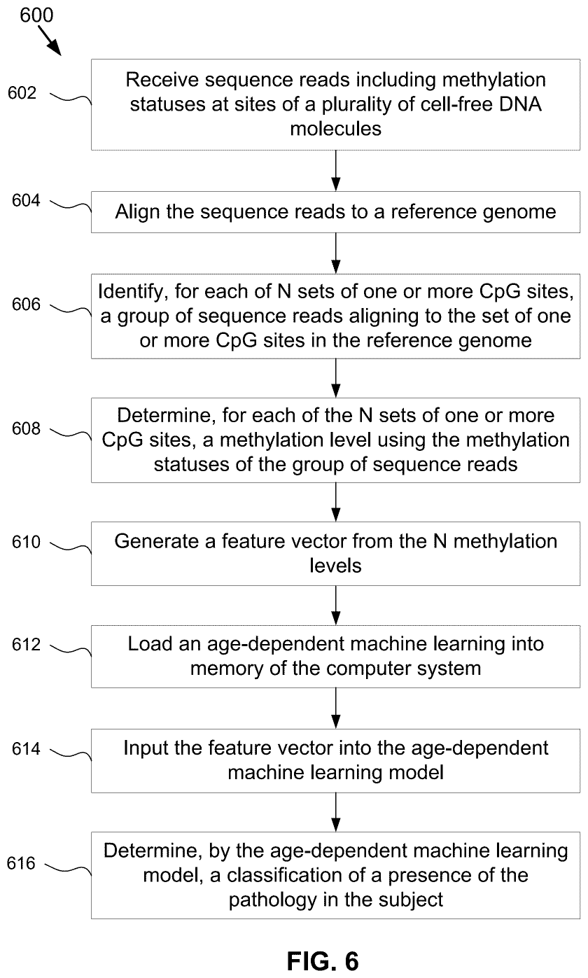

FIG. 6 shows a flowchart of an example of a method 600 for detecting a disorder in a subject having a known chronological age. Certain aspects of method 600 can be performed in a similar manner as method 400.

At block 602, the method 600 can include receiving sequence reads including methylation statuses at sites of a plurality of cell-free DNA (cfDNA) molecules. The sequence reads can include one or more sites for each of the cfDNA molecules. Block 602 can be performed in a similar manner as block 402 of method 400.

At block 604, the method 600 can include aligning the sequence reads to a reference genome. The reference genome can be a representative example of the DNA sequence of a species corresponding to the subject. The alignment can include searching for a location in the reference genome that best matches the sequence read.

The sequence reads may be aligned to N sets of one or more CpG sites in the reference genome. As examples, N is an integer that can be at least 3, 4, 5, 6, 7, 8, 9, 10, 11, 12, 13, 14, 15, 16, 17, 18, 19, 20, 30, 40, 50, 60, 70, 80, 90, or 100. Thus, there can be at least any of these numbers of CpG sites. Examples of CpG sites to which sequence reads may be aligned are provided in Table 4. Additionally or alternatively, the sequence reads may be aligned with genes in the reference genome. The genes may include any number of the 3,000 genes corresponding to the block dots depicted in FIG. 2 and provided in table 1. In some examples, at least some of the sets of one or more CpG sites may correspond to genes. Thus, at least some of the sequence reads may be aligned with sets of one or more CpG sites within a gene. Table 4 shows examples of CpG sites that correspond with genes. Block 604 can be performed in a similar manner as block 404 of method 400.

At block 606, the method 600 can include, for each of the N sets of one or more CpG sites, identifying a group of sequence reads aligning to the set of one or more CpG sites in the reference genome. For example, the reference genome can include annotations or other suitable indications of locations and boundaries of CpG sites, genes, or a combination thereof. Therefore, based on aligning the sequence reads with the reference genome, each sequence read can be associated with a CpG site, gene, or a combination thereof. Each sequence read may correspond with one or more CpG sites, genes, or the combination thereof, e.g., if the read is sufficiently long. Block 606 can be performed in a similar manner as block 406 of method 400.

Each of the CpG sites, genes, or the combination thereof may be associated with a particular tissue type, such as a particular organ. In one example, the particular tissue type can be selected from a group comprising bone marrow, brain, ovary, pancreas, liver, hypothalamus, heart, kidney, bladder, prostate, lymph nodes, breast, lung, skin, and testis. The association of each of the genes with the particular tissue type can be determined based on (1) a biological pathway or (2) epigenetic patterns or (3) expression levels in the particular tissue type being greater than a threshold.

At block 608, the method 600 can include, for each of N sets of one or more CpG sites, determining a methylation level using the methylation statuses of the group of sequence reads. Block 608 can be performed in a similar manner as block 408 of method 400.

For example, to determine a methylation level (e.g., a 5hmC level and/or a 5mC level) at the set of one or more CpG sites, an amount (number) of the methylation statuses at the set of one or more CpG sites that indicates a methylation is present or that indicates the methylation is not present may be determined. Thus, in some examples, a methylation level of a group of sequence reads aligning to a set of one or more CpG sites can be an amount of methylation statuses for each of the CpG sites in the set that indicate a methylation is present. Alternatively, in examples in which the sequence reads are aligned with genes in the reference genome, the methylation level may be a count (number) of methylated sites (or unmethylated sites) aggregated over the sequence reads for each gene. Thus, the methylation levels may be gene-specific methylation levels. Additionally, in examples in which at least some of the sets of one or more CpG sites correspond to genes, the methylation levels may be gene-specific methylation levels. For example, the methylation level may be an amount of methylation statuses for each CpG site corresponding to gene that indicates a methylation is present.

At block 610, the method 600 can include generating a feature vector from the N methylation levels. The feature vector can include the methylation level for each of the one or more CpG sites or for one or more genes. The feature vector can include the methylation levels in a structured form that can be ingested into and understood by a machine learning model. Block 610 can be performed in a similar manner as block 410 of method 400.

At block 612, the method 600 can include loading an age-dependent machine learning model into memory of the computer system. Various types and architectures of age-dependent machine learning models can be used. The age-dependent machine learning model can include a plurality of sub-models, each of which may correspond to a different biological age or age range.

At block 614, the method 600 can include inputting the feature vector into the age-dependent machine learning model. The feature vector (and other feature vectors described herein) can have various formats, e.g., organized serially as a single array or organized into a multi-dimensional as may be done when other values besides methylation levels are also included in the feature vector.

At block 616, the method 600 can include predicting, using the age-dependent machine learning model, a classification of a level of a pathology (e.g., presence of the pathology, such as a disorder) in the subject.

In some examples, determining the classification of the presence of the pathology in the subject can include comparing the feature vector to a representative reference vector determined using a group of the measured reference vectors that have a same known pathology classification (e.g., a disorder classification). A distance between the two vectors can be determined. If the feature vector is sufficiently similar to a first representative reference vector (e.g., distance within a threshold or is the closest representative reference vector), then the subject can be determined to have the first classification corresponding to the first representative reference vector.

The age-dependent machine learning model may generate a first representative reference vector based on reference vectors for sample subjects with the disorder (e.g., glioma) and a same or similar age to the subject, e.g., with a same age range. Thus, the first representative reference vector can include measured (including statistically determined, e.g., averaged) methylation levels for CpG sites or genes for sample subjects with the pathology.

Additionally, the age-dependent machine learning model can generate a second reference vector for healthy sample subjects (e.g., subjects without the pathology) of a same or similar age as the subject. Thus, predicting the classification of the presence of the disorder in the subject can include comparing the feature vector to each representative reference vector and determining which representative reference vector the feature vector is most similar to. In some examples, there can be any number of representative reference vectors corresponding to any number of pathologies.

Each chronological age or age range can have its own set of representative feature vectors. Thus, the model can be comprised of sub-models of different ages, where a sub-model has a set of representative feature vectors for different classifications. In other implementations, the model can determine a distance relative to all other representative reference vectors for all ages at once, but a distance to such reference vectors for a different age can have a weight that scales that dimension, such that the distance to reference vectors for different ages is generally higher than the distance to reference vectors for the same age.

Additionally in some examples, determining the classification of the presence of the disorder can include predicting, using the age-dependent machine learning model, a biological age of the subject, comparing the biological age to the known chronological age, and determining the classification of the presence of the disorder in the subject based on the comparison. For example, to determine the classification based on the comparison, the method 600 can include determining whether a difference between the biological age and the known chronological age exceeds a threshold. If the difference exceeds the threshold, the classification may indicate the subject has the disorder. The difference can be positive (age acceleration) or negative (age deceleration), each corresponding to a type of age aberration.

### V. GENES WITH SIMILAR SHAPE CHARACTERISTICS

As described in section I-III, there can be sets of CpG sites and/or genes for which methylation level (e.g., 5mC level and/or 5hmC level) varies over time. Typical methylation level patterns of change (e.g., methylation level trajectories) for sets of CpG sites and/or for genes with respect to age can be estimated based on methylation level data for subjects without cancer. The methylation level trajectories can then be used to gain insight into biological processes (e.g., disease progression or aging).

### A. Methylation Level Trajectory During Ageing

To estimate methylation level trajectories (e.g., 5hmC level trajectories and/or 5mC level trajectories) during ageing, cfDNA methylation levels can be z-scored. For example, the cfDNA 5hmC levels for each gene across the first dataset, the second dataset, or the combination thereof can be z-scored. Alternatively, methylation levels for sets of CpG sites (e.g., sets of the CpG sites in table 4) can be z-scored. For example, methylation levels for sets of CpG sites as obtained via whole-genome bisulfite sequencing of plasma cfDNA samples from the 136 subjects without cancer can be z-scored.

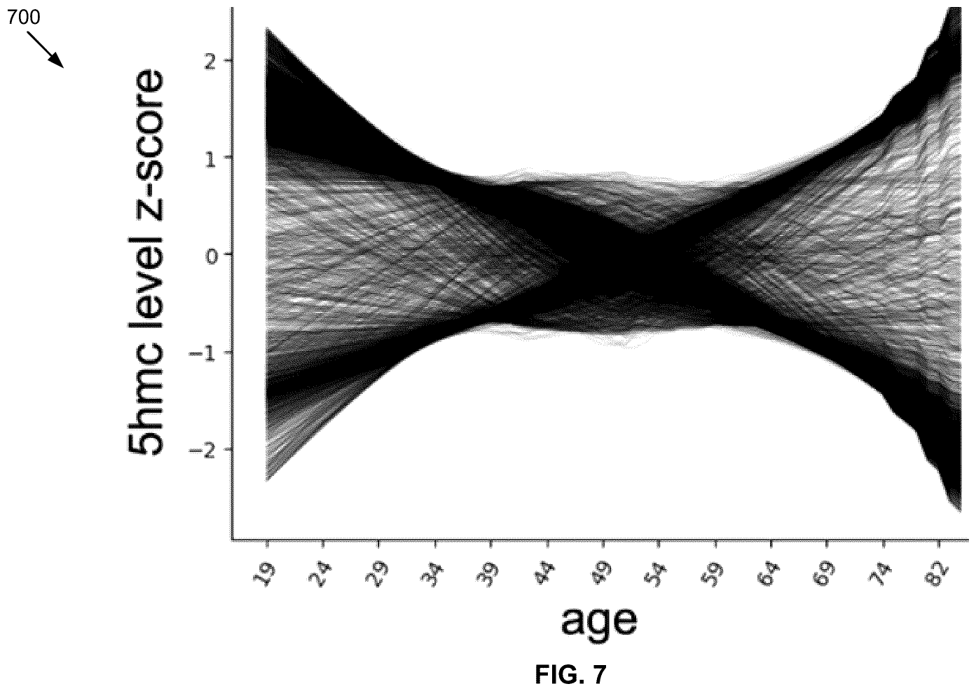

FIG. 7 shows the 5hmC level z-scores for each highly variable gene identified based on the cohort. The 5hmC level z-score for each highly variable gene for each subject is plotted against an age of the subject. As a result, FIG. 7 shows a plot 700 of cfDNA 5hmC trajectories for the highly variable genes during ageing, according to some embodiments of the present disclosure.

Additionally, locally estimated scatterplot smoothing (LOESS) regression can be fitted to each set of CpG sites and/or to each gene to estimate each set of CpG site's or each gene's methylation level trajectory with age. Additionally or alternatively, methylation level trajectories can be established based on various smoothing algorithms, such as but not limited to LOESS, bin smoothing, moving average, splines, penalized splines, wavelets, Savitsky-Golay filter, etc. The methylation level trajectories established using the smoothing algorithms can show diverse patterns of change (e.g., trajectories) for some sets of CpG sites and/or genes with age. In some examples, once the methylation level trajectories are established, sets of CpG sites or genes with similar methylation level trajectories can be grouped. Set of CpG sites or sets of genes with similar 5hmC trajectories may be grouped by performing unsupervised clustering with fuzzy C-means (FCM). In one embodiment, clustering methylation level trajectories can be conducted using various clustering algorithms, such as but not limited to FCM, k-means clustering, hierarchal clustering, principal component analysis (PCA), AutoEncoder, Deep Belief Network (DBN), Restricted Boltzmann Machine (RBM), Hierarchical Temporal Memory (HTM), Convolutional Neural Networks (CNNs), Support Vector Machines (SVMs), singular value decomposition (SVD), non-negative matrix factorization (NMF), etc.

In an example, the highly variable genes identified from the data for the cohort can be grouped into clusters based on corresponding 5hmC trajectories. To do so, the clustering method FCM can be used. The clustering method can group genes with similar trajectories into clusters and can output a membership value for each gene. The membership value for each gene can indicate a degree of similarity of the gene with the cluster in which it is grouped. A cutoff membership value can be used to select genes with high relevance to the clusters. For example, the cutoff membership value can be one. As a result, in the example, 1,926 of the 3,000 highly variable genes can be grouped into eight cluster.

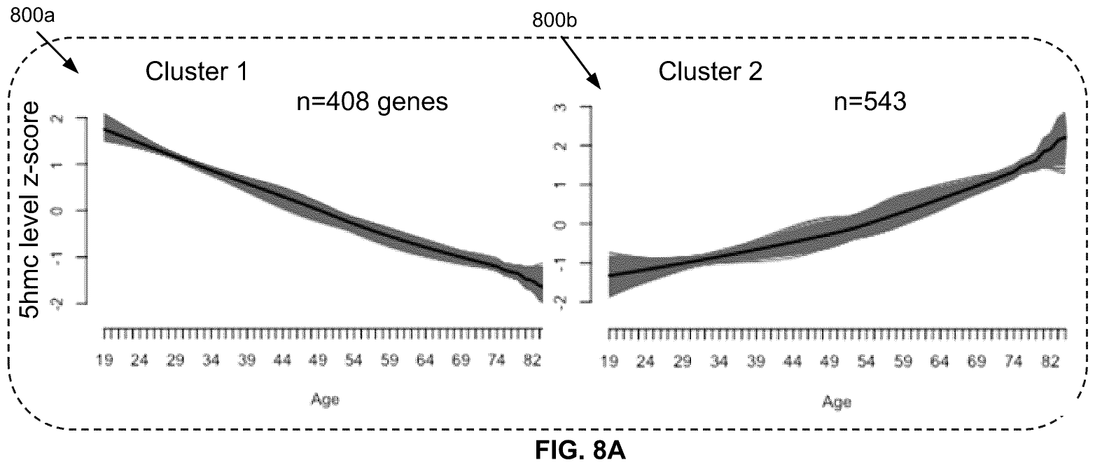

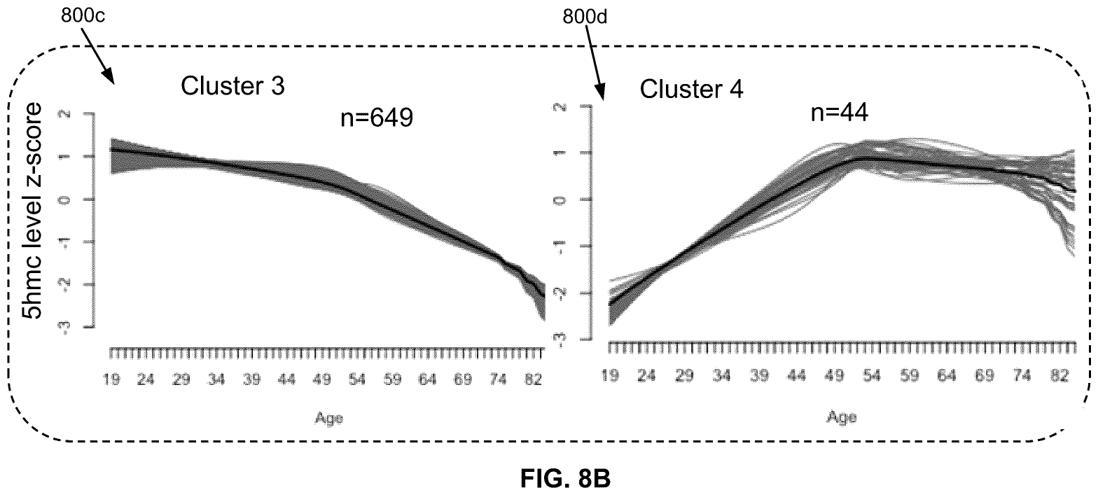

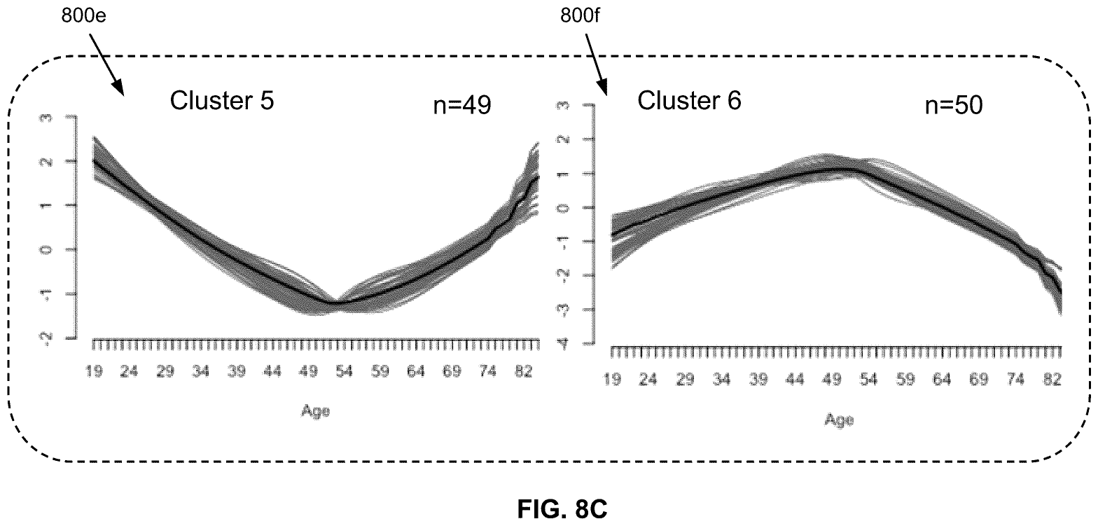

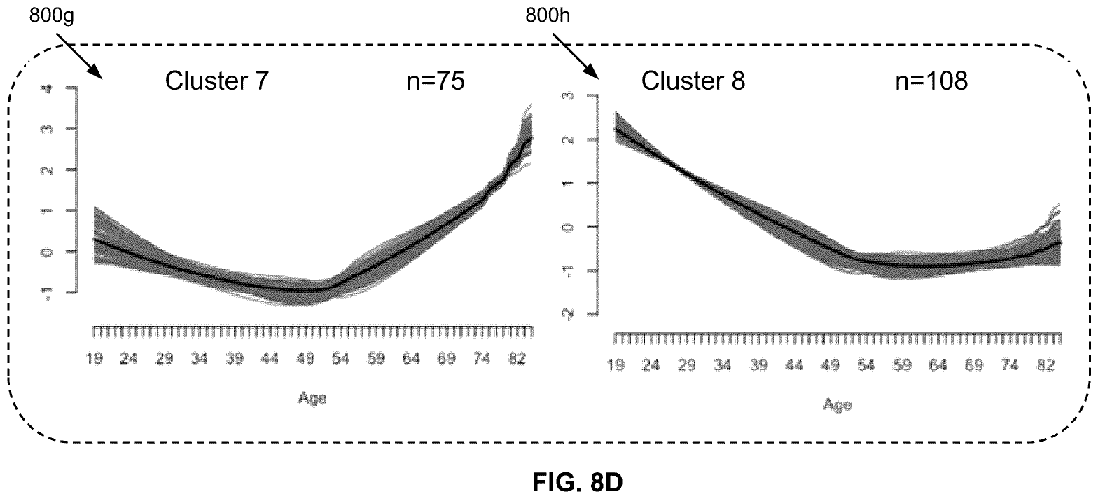

FIGS. 8A-8D depict plots 800a-h of the eight gene clusters identified in the example. The eight gene clusters can each be characterized by 5hmC trajectories with similar shape characteristics. Four shape classifications are shown, but more classifications can be used. The similar shape characteristics can include similar slopes, similar directions of the slopes (e.g., positive or negative), similar general shapes (e.g., linear, exponential, quadratic, or logarithmic), or a combination thereof. The FIGS. 8A-8D can further show the 5hmC trajectories for the genes in each cluster across a range of ages (e.g., from nineteen to eighty-two). For example, the 5hmC trajectories can be depicted on plots 800a-h in FIGS. 8A-8D. Each plot 800a-h can include the 5hmC level z-score for each gene in the corresponding cluster. The 5hmC level z-scores can be plotted against true ages for each subject corresponding to each gene. The clusters may range in size with respect to a number of associated genes.

As depicted in FIG. 8A, a first cluster and a second cluster can include genes with 5hmC trajectories that are substantially linear, while the other six clusters may include genes with 5hmC trajectories that are substantially non-linear. For example, as shown in FIG. 8B, a third cluster and a fourth cluster can include genes with 5hmC trajectories that are substantially logarithmic. Additionally, as shown FIG. 8C, a fifth cluster and a sixth cluster can include genes with 5hmC trajectories that are substantially quadratic. Moreover, as shown in FIG. 8D, a seventh cluster and an eighth cluster can include genes with 5hmC trajectories that are substantially exponential. The black line in each of the plots 800a-h can represent a mean for all the genes in each cluster.

The classification of which shape classification applies can be done in various ways. For example, the data points for a methylation level of a gene for samples taken at different stages can be fit to a line, and if the accuracy (e.g., r value) is within a threshold, then the shape classification can be linear. Similar operations can be done for other functional forms.

### B. Relationship Between Gene Clusters and Biological Phenotypes

Gene set enrichment analysis (GSEA) can be performed to determine whether the gene clusters are linked to one or more biological phenotypes. For example, the GSEA analysis can include determining whether the genes for each cluster are associated with an enriched biological pathway, an enriched tissue, or a combination thereof. The enriched biological pathway, the enriched tissue, or the combination thereof for each cluster can be identified using a reactome database (e.g., including biological pathways), a MalaCards database (or other databases that provide genes and their relationship to diseases), other suitable databases, or a combination thereof.

Based on the GSEA it can be determined that the genes in the first cluster are represented in GPCR signaling and neuron-associated pathways. The biological pathways associated with the first cluster can be consistent with an enriched tissue type determined for the first cluster, which can be the brain. Additionally, the GSEA of cluster two can indicate that the corresponding genes are associated with immune and hemostasis pathways and related tissue, such as blood and bone marrow. The genes corresponding to the third cluster can be involved in metabolism (e.g., biological oxidation) and drug ADME pathways and related tissue such as the liver and kidney. Genes associated with the fifth cluster can also be associated with metabolism and may particularly show enrichment in lipid metabolism pathways and endothelial tissues. Genes in the seventh cluster can be skin-related. For example, based on the GSEA it can be determined that genes in the seventh cluster can be associated with collagen pathway and dermis tissue enrichments. The eighth cluster can include genes related to the signaling pathways of ERBB, which can be a family of proteins (e.g., EGFR and HER2) associated with development of neurodegenerative diseases and some cancer. Additionally, based on the GSEA analysis it can be determined that genes in each of the eight clusters share enrichment in the brain or neuron tissues, which may suggest a key role of 5hmC in neurodevelopment.

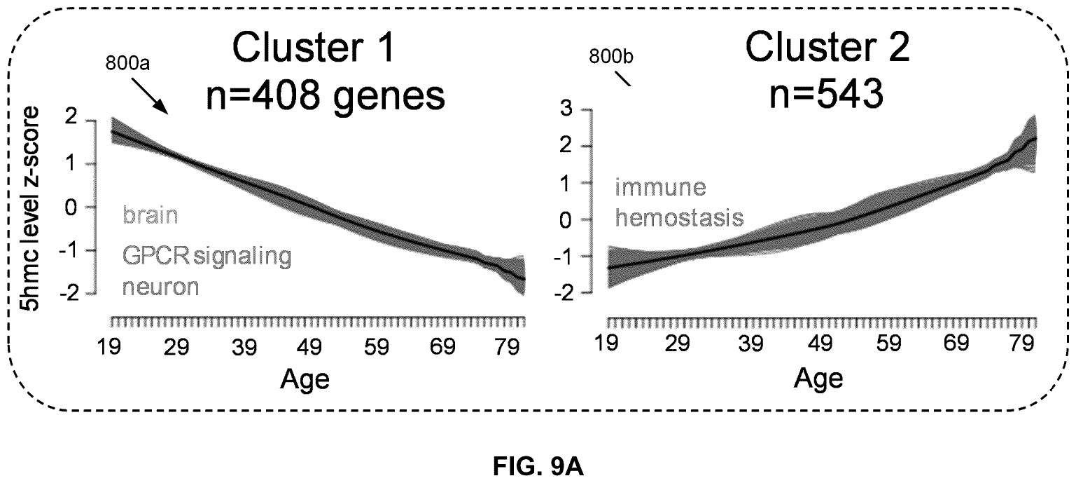

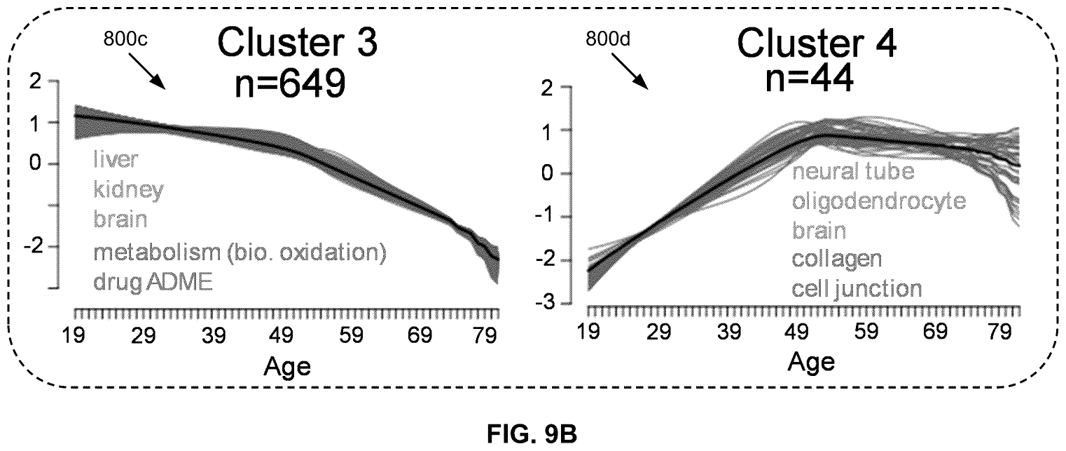

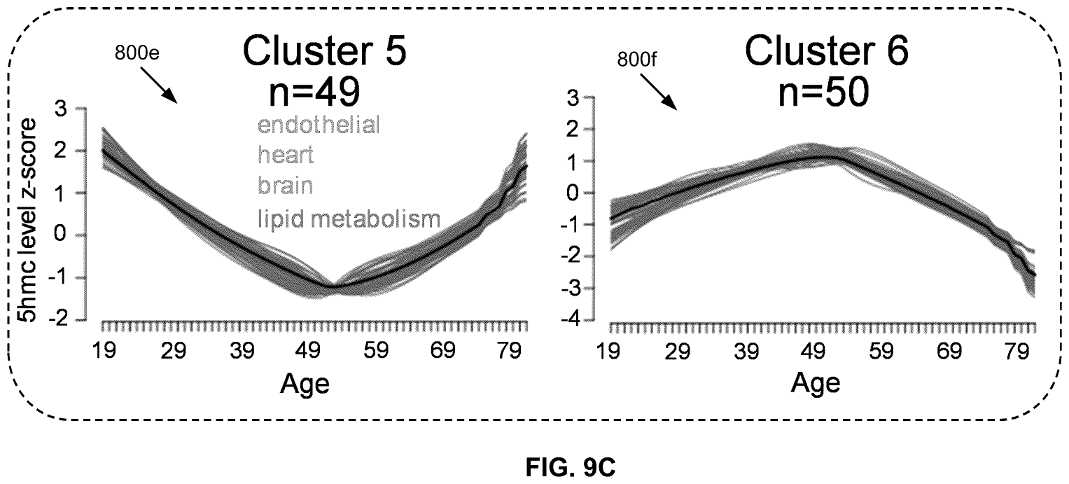

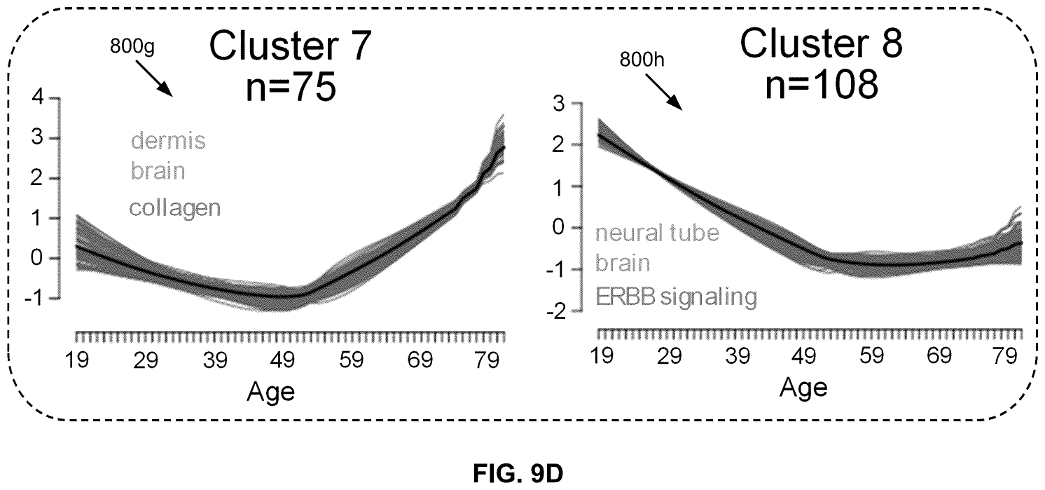

FIGS. 9A-D shows the plots 800a-h of the gene-specific cfDNA 5hmC trajectories, according to embodiments of the present disclosure. That is, FIGS. 9A-D show the 5hmC trajectories of genes in each of the clusters identified used the FCM algorithm. FIGS. 9A-D further includes an indication of the enriched biological pathways and enriched tissues determined for the genes in each of the clusters.

### C. Gene Clusters and Age Indicators

In addition to clusters being related to particular biological pathways and tissue, there can be periods of aging associated with the clusters that exhibit changes in the methylation level trajectories. The periods of aging can be indicated by changing points or turning points in the methylation level trajectories. The changing or turning points can be evident for many or most methylation level trajectories for sets of CpG sites and/or genes in a cluster. A turning point can be characterized by an increase or decrease in slopes of methylation level trajectories for sets of CpG sites and/or genes in a cluster. Additionally, a changing point be characterized by a change in direction of the slopes of methylation level trajectories for sets of CpG sites and/or genes in a cluster (e.g., from positive to negative or from negative to positive).

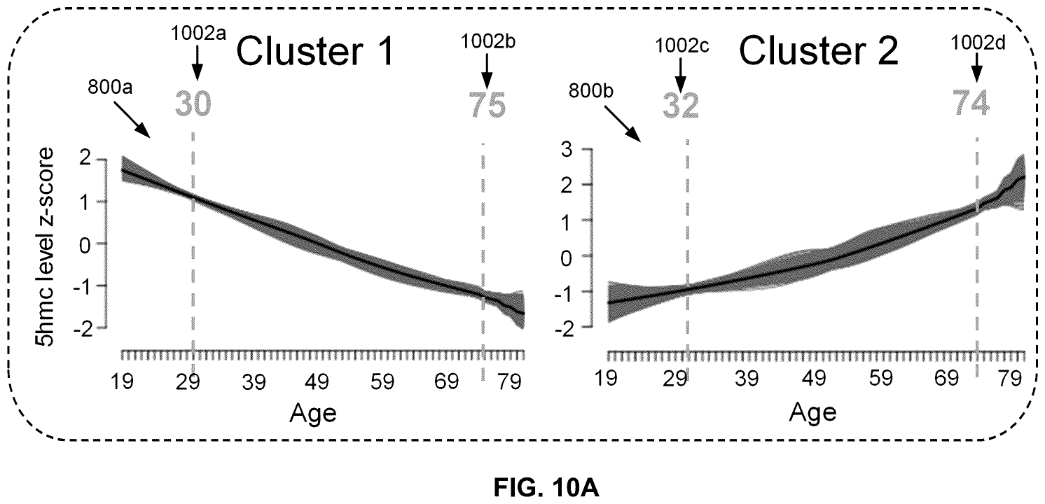

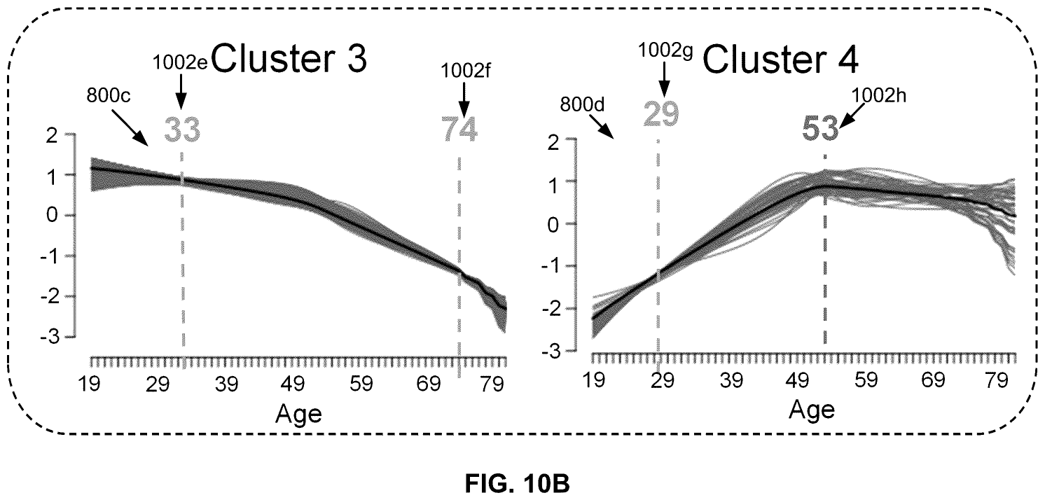

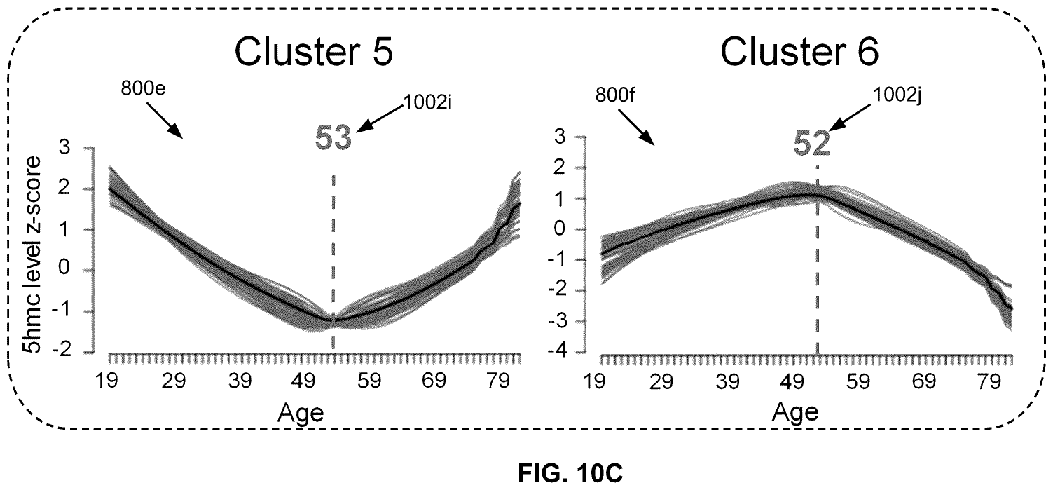

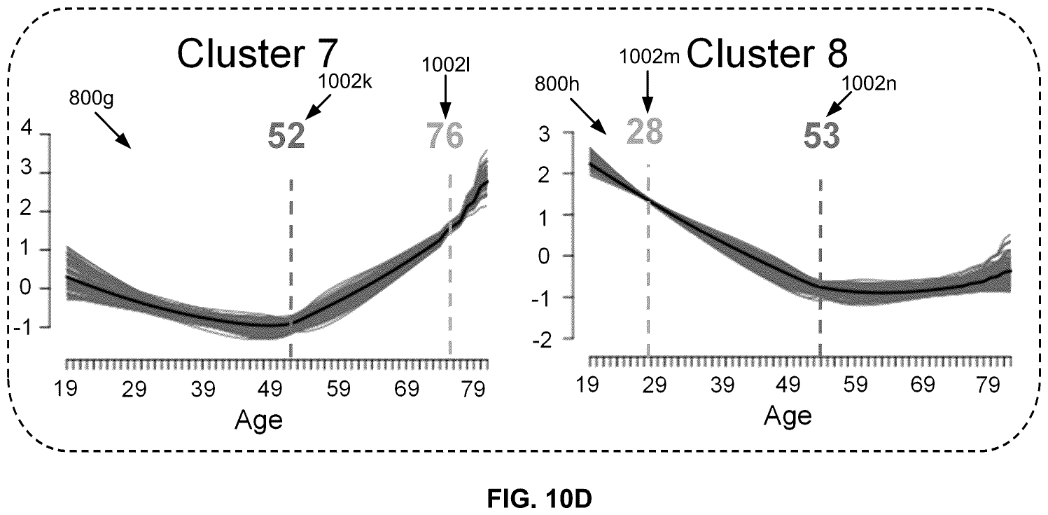

FIG. 10A shows the plots 800a-b of gene-specific cfDNA 5hmC trajectories with age indicators 1002a-d. FIG. 10B shows the plots 800c-d of gene-specific cfDNA 5hmC trajectories with age indicators 1002e-h. FIG. 10C shows the plots 800e-f of gene-specific cfDNA 5hmC trajectories with age indicators 1002i-j. FIG. 10D shows the plots 800g-h of gene-specific cfDNA 5hmC trajectories with age indicators 1002k-n. The age indicators 1002a-n can be positioned at the changing points or the turning points for each cluster.

For example, as depicted in a plot 800a, variance of 5hmC trajectories for genes in a first cluster can decrease around age 30. The change in variance of the 5hmC trajectories around age 30 can be labelled with a first age indicator 1002a. The slopes of the 5hmC trajectories in the first cluster can also decrease around age 75 as is labelled with a second age indicator 1002b. The decrease in variance can indicate that there is less variation in 5hmC levels for genes in subjects around the ages of 30 and 75. Similar decreases in variance of 5hmC trajectories can also be observed for subjects around age 30 the second cluster (e.g., as depicted in plot 800b), the third cluster (e.g., as depicted in plot 800c), the fourth cluster (e.g., as depicted in plot 800d), and the eighth cluster (e.g., as depicted in plot 800h). Additionally, the decreases in variance around age 75 can be observed in the first cluster, the second cluster, and the seventh cluster (e.g., as depicted in plot 800g). Thus, based on the changes in 5hmC trajectories for the first cluster, second cluster, third cluster, fourth cluster, seventh cluster, and the eighth cluster, the 30's and 70's can be identified as age periods with low gene-wise dispersion with respect to 5hmC levels.

Additionally, the fourth cluster, fifth cluster, sixth cluster, seventh cluster, and eighth cluster can have changing points around the age of approximately 50. For example, as shown at an eighth age indicator 1002h and a tenth age indicator 1002j, the 5hmC level in the fourth cluster and in the sixth cluster can increase until around the age of 50. Then, after the age of 50, 5hmC levels for genes in the fourth cluster and the sixth cluster can decrease. The increase or decrease in 5hmc level for genes can be indicated by positive or negative slopes of the 5hmC trajectories respectively. Further, as indicated by a ninth age indicator 1002i, an eleventh age indicator 1002k, and a fourteenth age indicator 1002n, 5hmc levels in genes in the fifth, seventh, and eighth clusters can decrease in subjects up to 50 years old. Then, the 5hmC levels for the genes in the fifth, seventh, and eighth clusters can increase in the subjects older than fifty.

Therefore, based on the 5hmC trajectories of the clustered genes, the periods of ageing in which 5hmC levels change in variation may be around the 30's, 50's, and 70's.

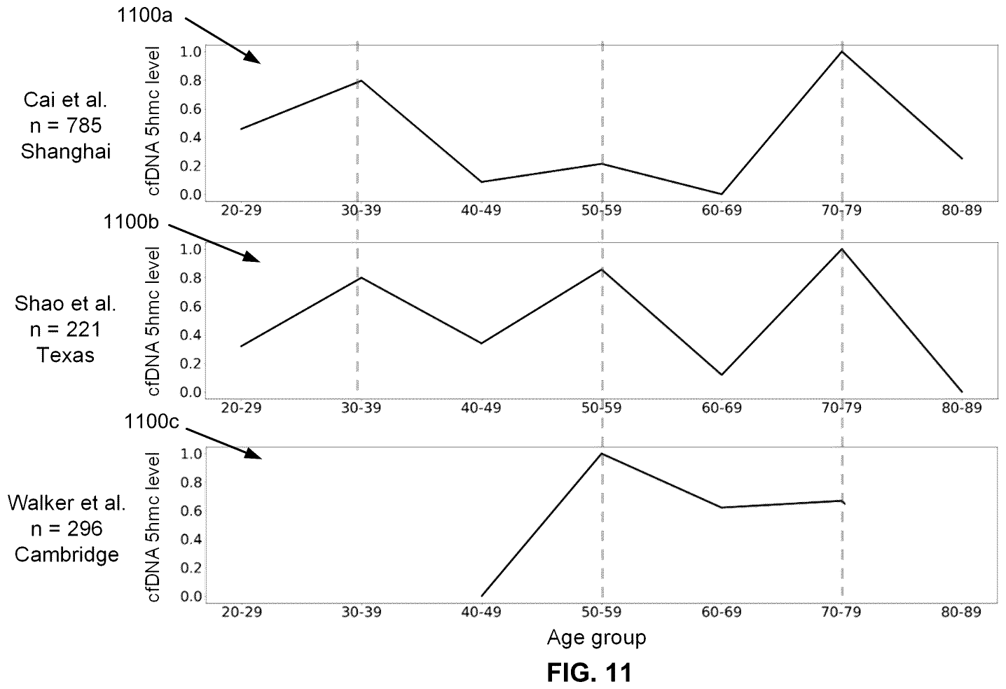

FIG. 11 also shows plots 1100a-c of cfDNA 5hmC trajectories during ageing, according to embodiments of the present disclosure. The cfDNA 5hmc level trajectories depicted in FIG. 11 can be representative of overall cfDNA 5hmc levels for subjects of different ages rather than representative of gene-specific 5hmC levels. As shown in FIG. 11, whole-genome 5hmC levels for cfDNA may exhibit crests for subjects in the 30s, 50s, and 70s. The crests can further be confirmed with data for two additional cohorts (Shao et al., 2022; Walker et al., 2022). The whole-genome 5hmC levels from the two additional cohorts are shown in plots 1100b-c.

### D. Organ-Specific Aging Analysis

Plasma cfDNA analysis can further allow for the assessment of organ aging patterns. In some embodiment, in addition to blood cells, cells from the liver, lung, colon, or other suitable organs can be used in organ specific aging analysis. To perform organ-specific aging analysis, tissue-specific methylation trajectories (e.g., tissue-specific 5hmC trajectories) can be determined. For example, a group of genes with a same shape characteristic can be determined, and an associated tissue can be identified as described herein. Then, using the tissue-specific methylation trajectories, a subject's biological age or a biological age of a corresponding pathway can be predicted.

### 1. Example Model for Organ-Specific Age Prediction

A biological age associated with a specific organ or specific biological pathways may be estimated using cfDNA methylation level data. For example, as determined using the GSEA and as shown in FIG. 9, the first and eight clusters (e.g., corresponding with plot 800a and 800h) can include genes enriched in brain or neural tissues. Therefore, the genes corresponding to the first and eighth clusters can be considered brain-specific genes. In the example shown for FIG. 9, between the first and eighth clusters, there are 516 genes corresponding to the brain or neural tissues.

In the example shown in FIG. 9, the first and eight clusters were established via unsupervised clustering with fuzzy C-means (FCM). In other embodiments, brain-specific or other organ-specific genes may be defined in a supervised way. For example, brain-specific genes can be defined as the genes showing differential 5hmC patterns in the brain relative to other tissues. In another example, brain-specific genes can be defined as the genes showing differential expression, methylation, or histone modification patterns in the brain relative to other tissues. For example, organ-specific genes with linear changes in methylation levels can be selected.

Using cfDNA 5hmC data corresponding to the genes in the first and eighth clusters, a brain-specific ageing model can be generated. In an example, the brain-specific aging model can use LASSO regression to predict age based on the cfDNA 5hmC data. The cfDNA 5hmc data can include 5hmC levels for each of the genes in the first and eighth clusters. The cfDNA data can further be from the subjects without cancer in the first dataset, and can be split into a training dataset and a testing dataset with, for example, a 9:1 ratio.

In an example, brain-specific genes with detectable 5hmC signals in the second dataset may be used to train and test the brain-specific ageing model. Thus, in the example, 462 of the genes from the first and eighth clusters can be used as input features for the brain-specific ageing model. During training, in the example, brain-specific ageing model finally may select a portion (e.g., 180) of the genes usable to predict biological brain age.

After training, 5hmc levels for the selected genes from the testing dataset can be input into the brain-specific aging model. The brain-specific aging model can then output predicted ages, which may correlate with true ages of the corresponding subjects without cancer.

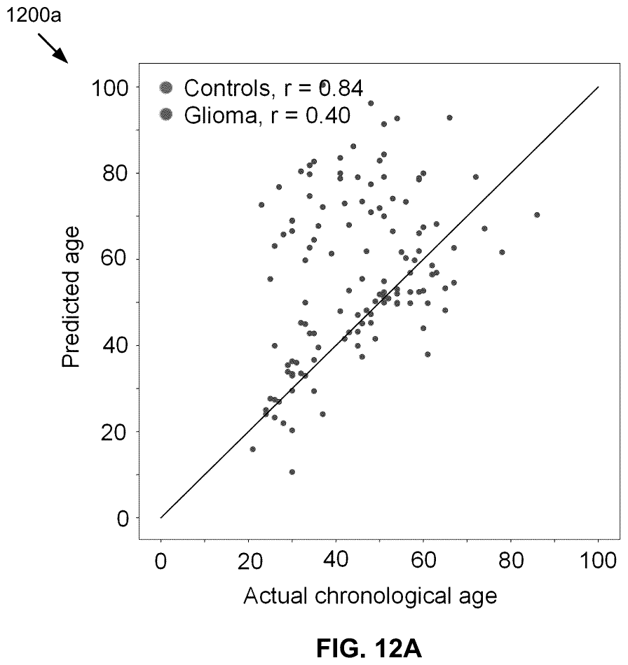

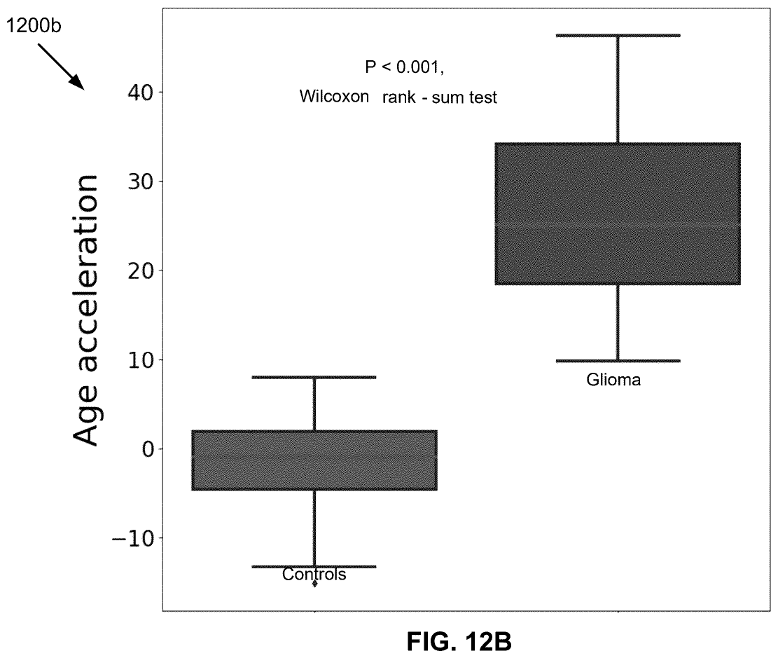

FIG. 12A shows a plot of brain-specific age prediction against true chronological ages, according to some embodiments of the present disclosure. In particular, FIG. 12A can include the predicted ages for controls (e.g., subject without cancer in the testing dataset) vs true ages of the controls. The predicted ages from the brain-specific aging model may strongly correlate with the true ages. For example, the Pearson's coefficient for the predicted and true ages can be 0.84.

Additionally, after training, 5hmc levels for the selected genes from the second dataset can be input into the brain-specific aging model. Thus, the model can be applied for the subjects with glioma. The brain-specific aging model can then output predicted ages for the subjects with glioma based on the 5hmC levels for the selected genes from the second dataset. The predicted ages for the subjects with glioma can have a low correlation with the true ages of the subjects. For example, the Pearson's coefficient can be 0.40.

Furthermore, as shown in FIG. 12A, the predicted ages for the subjects with glioma can be generally greater than the true ages. Thus, it can be predicted that subjects with glioma may exhibit accelerated aging with respect to controls. A Wilcoxon rank-sum test can be performed to determine whether the age acceleration for the subjects with glioma is significantly different from age acceleration for the subjects without cancer.

FIG. 12B shows a box plot of organ-specific age acceleration, according to some embodiments of the present disclosure. As shown in FIG. 12B, a median age acceleration for the brain-specific age predictions for the subjects with glioma is 25.07, while the median age acceleration for brain-specific age predictions for the subjects without cancer is −0.99 years. Additionally, a p value below 0.001 from the Wilcoxon rank-sum test can indicate a significant difference between the age accelerations.

In comparison with FIGS. 5A-5B, the median age acceleration associated with the brain-specific age predictions can be greater than the median age acceleration for the age prediction without organ-specificity. This may suggest that a brain-specific ageing clock (e.g., the brain-specific aging model) can contain enhanced information related to the impact of glioma on brain or neural tissue. 2. Organ-Specific Ageing Clocks

Comparisons between biological and chronological ages for a particular organ can allow for the determination of the acceleration or deceleration in the organ's biological age, which might enable non-invasive disease detection and preventative interventions associated with this organ. To further demonstrate the feasibility of building ageing clocks for individual organs, organ-specific ageing models for K different organs were constructed using organ-specific genes with elevated 5hmC levels. As examples, K can beat least 5, 6, 7, 8, 9, 10, 11, 12, 13, 14, 15, 16, 17, 18, 19, 20, 21, 22, 23, or more organs. For example, brain-specific genes can refer to the genes with high methylation levels (e.g., high 5hmC levels) in brain tissue relative to other tissue types. In another example, organ- or tissue-specific genes can be defined as the genes showing differential 5mC, methylation, expression, or histone modification patterns in a particular organ or tissue relative to other organs or tissues.

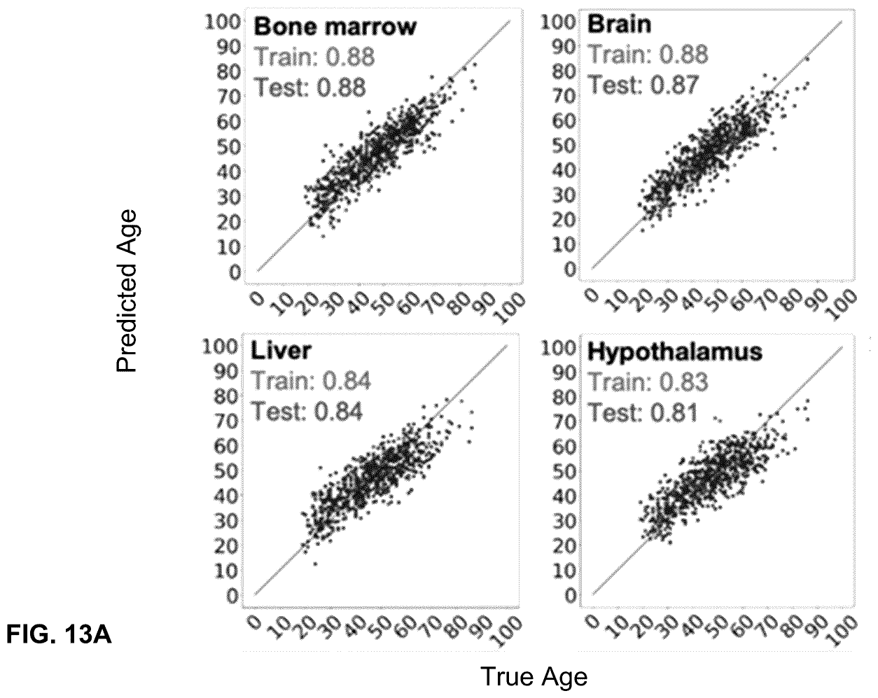

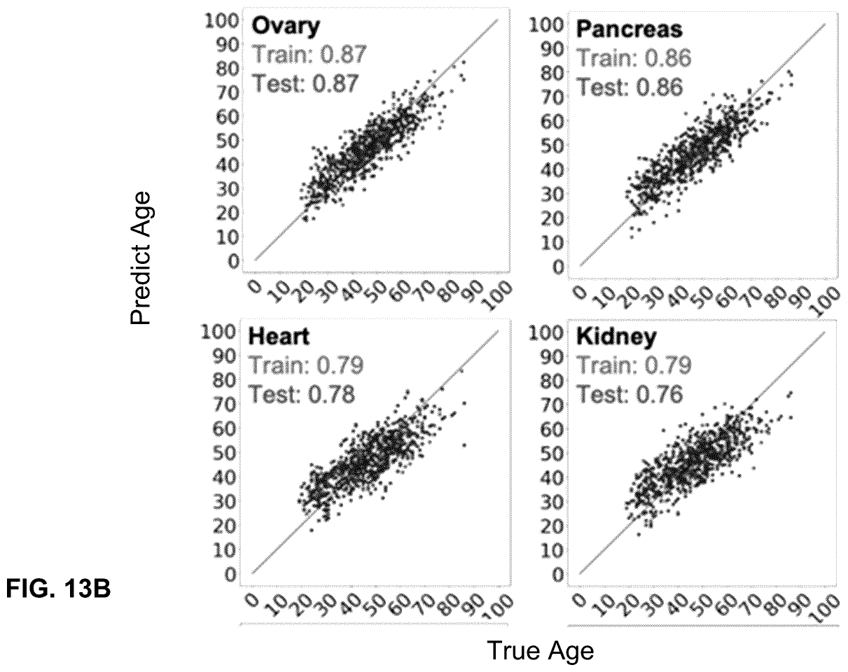

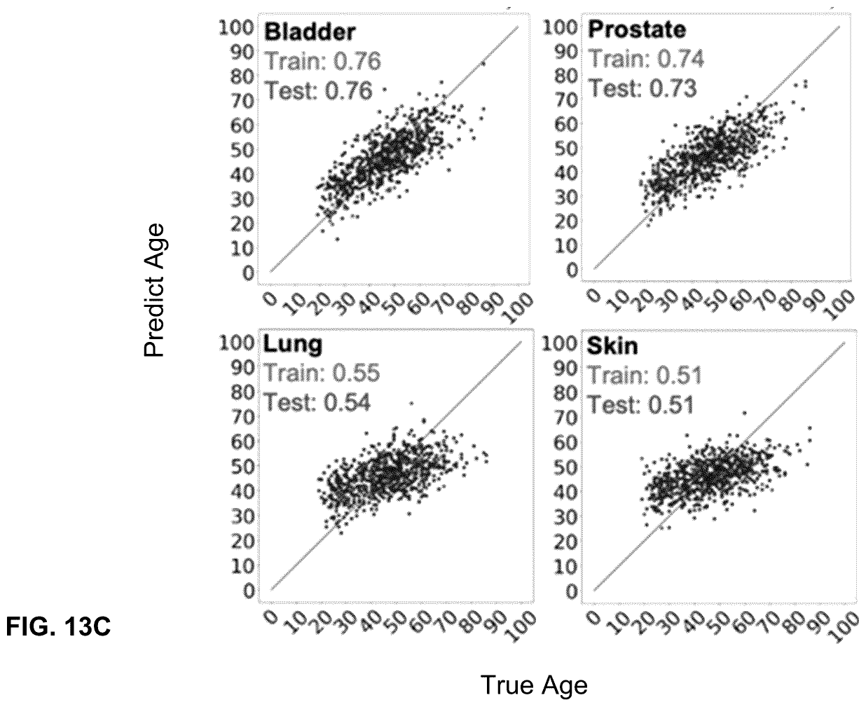

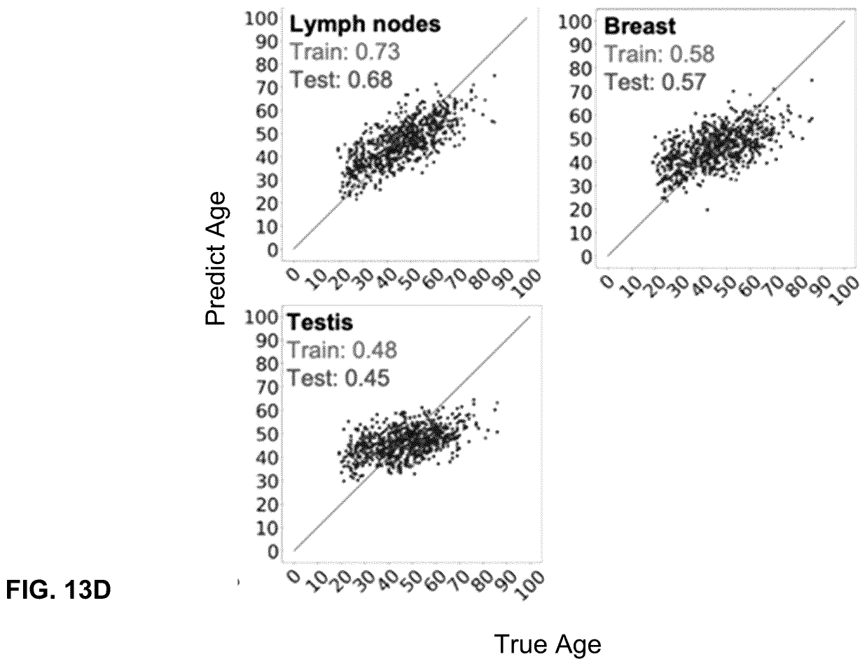

In this example, K (e.g., 15) organ-specific ageing models were developed using LASSO regression based on the cfDNA 5hmC data for the subjects without cancer in the first dataset. Similar to the brain-specific aging model, each organ-specific model can take cfDNA 5hmc data for genes enriched in biological pathways, enriched in tissue, or otherwise associated with the organ. The cfDNA 5hmC data for each of the genes corresponding to each organ can be split into a training dataset and a testing dataset. The predicted ages provided by each organ-specific model using the testing and training datasets for each organ can be depicted in FIGS. 13A-13D. The predicted ages in FIGS. 13A-13D can be plotted against true ages of the subjects without cancer from the first dataset.

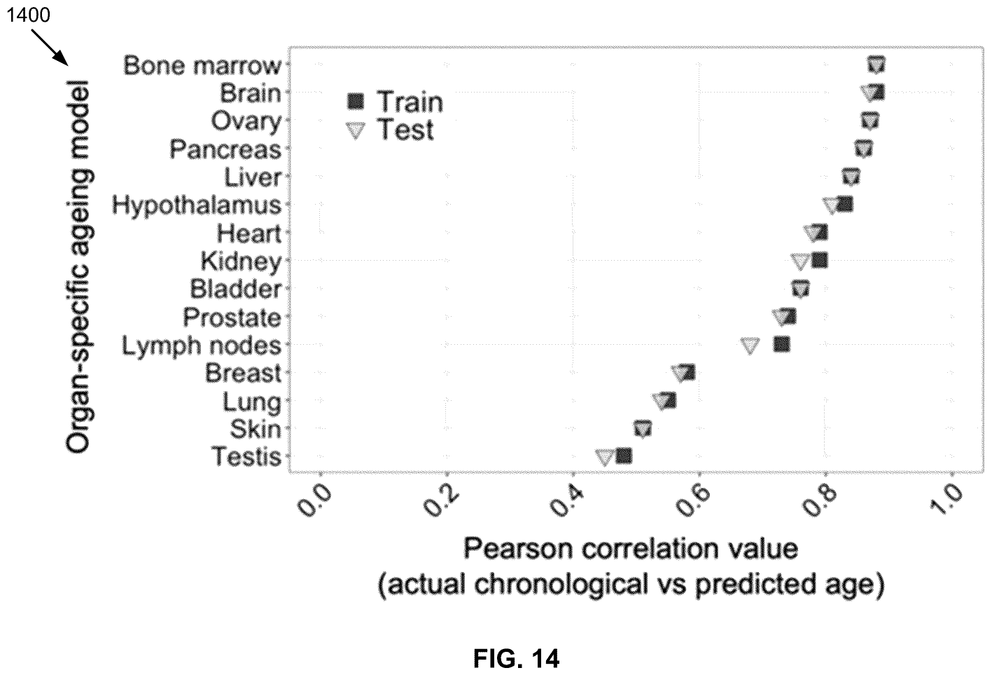

An accuracy of the predicted ages for each organ-specific model with respect to the true ages for the subjects without cancer can be estimated using Pearson's coefficient. FIG. 14 shows a plot 1400 of the Pearson correlation values for each of the organ-specific age models, according to some embodiments of the present disclosure. These organ-specific models can provide insight into aging processes at the organ level, which may allow for more precise assessments of aging processes or disease progression.

In some embodiments, an age can be determined for two or more organs. Such organ ages can be combined to determine a spectrum or landscape of organ-specific ages from different age-specific models (e.g. liver age, brain age, etc.). An overall age from various genes can also be determined and used in the spectrum (e.g., a set) of ages. The person's age spectrum can be compared to the age spectrum of a healthy person or a person with a known pathology.

### E. Method Using Shape Characteristics

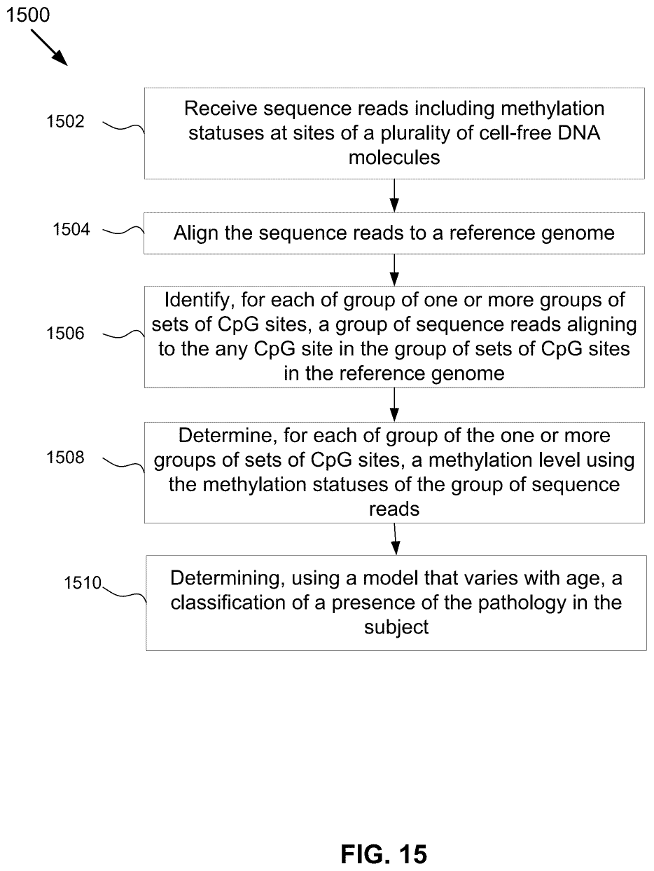

FIG. 15 is a flowchart illustrating a method 1500 for detecting a pathology (e.g., a condition or disorder) in a subject having a known chronological age, according to some embodiments of the present disclosure. Certain aspects of method 1500 can be performed in a similar manner as methods 400 and 600.

At block 1502, the method 1500 can include receiving sequence reads including methylation statuses at sites of a plurality of cell-free DNA (cfDNA) molecules. The sequence reads can include one or more sites for each of the cfDNA molecules. Block 1502 can be performed in a similar manner as block 402 of method 400.

At block 1504, the method 1500 can include aligning the sequence reads to a reference genome. The reference genome can be a representative example of the DNA sequence of a species corresponding to the subject. The alignment can involve searching for a location in the reference genome that best matches the sequence read.

The sequence reads may be aligned to sets of CpG sites in the reference genome. Examples of CpG sites to which sequence reads may be aligned are provided in Table 4. Additionally or alternatively, the sequence reads may be aligned with genes in the reference genome. The genes may include any number of the 3,000 genes corresponding to the block dots depicted in FIG. 2 and provided in table 1. In some examples, at least some of the sets of one or more CpG sites may correspond to genes. Thus, at least some of the sequence reads may be aligned with sets of CpG sites within a gene. Table 4 shows examples of CpG sites that correspond with genes. Block 1504 can be performed in a similar manner as block 404 of method 400.

At block 1506, the method 1500 can include, for each group of one or more groups of sets of CpG sites, identifying a group of sequence reads aligning to any CpG site in the group of sets of CpG sites. The group of sets of CpG sites may include at least 3 sets of CpG sites. In some examples, a set of CpG sites may include at least 4, 5, 6, 7, 8, 9, 10, 11, 12, 13, 14, 15, 16, 17, 18, 19, 20, 30, 40, 50, 60, 70, 80, 90, 100, 200, or 250 CpG sites. Moreover, in some examples, a group of sets of CpG sites may include at least 4, 5, 6, 7, 8, 9, 10, 11, 12, 13, 14, 15, 16, 17, 18, 19, 20, 30, 40, 50, 60, 70, 80, 90, or 100 sets of CpG sites.

Additionally, each set of CpG sites in the group may have a same shape classification for a change in a methylation level with respect to age. The shape classification can be selected from a group consisting of linear, logarithmic, quadratic, and exponential. Thus, each CpG site in each set of CpG sides in the group can exhibit similar changes in methylation levels over time in a manner which may be substantially linear, logarithmic, quadratic, or exponential.

Additionally or alternatively, as mentioned above, one or more sets of CpG sites may correspond to a gene. In such examples, a group of sets of CpG sites may correspond to a group of genes where each gene in each group may have a same shape classification for a change in a methylation level with respect to age. The shape classification can be selected from a group consisting of linear, logarithmic, quadratic, and exponential. That is each gene in the group of genes can exhibit similar changes in methylation levels over time in a manner which may be substantially linear, logarithmic, quadratic, or exponential. For example, the groups of genes can correspond to the clusters discussed above with respect to FIGS. 8-10.

In some examples, each group of sets of CpG sites and/or each gene corresponding to each group of sets of CpG sites can be associated with a particular tissue The association of each set of CpG site or each gene with a particular tissue can be determined based on (1) a biological pathway or (2) epigenetic patterns or (3) expression levels in the particular tissue type being greater than a threshold. The particular tissue type can be an organ. More specifically, the tissue type can include bone marrow, brain, ovary, pancreas, liver, hypothalamus, heart, kidney, bladder, prostate, lymph nodes, breast, lung, skin, or testis.

At block 1508, the method 1500 can include, for each of one or more groups of sets of CpG sites, determining one or more methylation levels using the methylation statuses of the group of sequence reads. In some examples, the methylation levels can be a number of methylated sites for each group of sets of CpG sites for which a group of sequence reads was aligned. Alternatively, in some examples, the methylation level can be a proportion, percentage, or other suitable quantification of a concentration of methylated sites for each group of sets of CpG sites. That is, the methylation levels can be a measure of a number of methylated sites for each group of sets of CpG sites with respect to a total number of sites in group. As examples, the number of groups of sets of CpG sites (e.g., genes) can be at least 2, 3, 4, 5, 6, 7, 8, 9, 10, 15, 20, 30, 40, or 50. Each gene can comprise various numbers of CpG sites, e.g., numbers listed about for other methods described herein.

For example, to determine a methylation level (e.g., a 5hmC level and/or a 5mC level) of a group of sets of CpG sites, an amount (number) of the methylation statuses at the set of one or more CpG sites that indicates a methylation is present or that indicates the methylation is not present may be determined. Thus, in some examples, a methylation level of a group of sequence reads aligning to a group of sets of CpG sites can be an amount of methylation statuses for each of the CpG sites in the group that indicate a methylation is present.

Alternatively, in examples in which at least some of the sets of one or more CpG sites correspond to genes, the methylation levels may be gene-specific methylation levels. For example, the methylation level may be an amount of methylation statuses for each CpG site corresponding to gene that indicates a methylation is present.

At block 1510, the method 1500 can include, determining, using a model that varies with age, a classification of a presence of the pathology in the subject. Determining the classification of the presence of the pathology can use the known chronological age of the subject and the one or more methylation levels. The model can be an age-dependent machine learning model. The age-dependent machine learning model may include a plurality of sub-models, each corresponding to a different chronological age. The model can be generated using reference samples of subjects having known classifications for the pathology.

In one example, determining the classification can include comparing the one or more methylation levels to one or more thresholds, where the one or more thresholds are dependent on the known chronological age. For example, a normal range for a given chronological age (e.g. within a specified number of standard deviations) can be used for the one or more thresholds.

In some examples, determining the classification of the presence of the pathology in the subject can include generating a feature vector from the one or more methylation levels of the groups of sets of CpG sites and comparing the feature vector to a representative reference vector determined using a group of the measured reference vectors that have a same known pathology classification (e.g., a disorder classification). A distance between the two vectors can be determined. If the feature vector is sufficient similar to a first representative reference vector (e.g., distance within a threshold or is the closest representative reference vector), then the subject can be determined to have the first classification corresponding to the first representative reference vector.

The model may generate a first representative reference vector based on reference vectors for sample subjects with the pathology (e.g., glioma) and a same or similar age to the subject, e.g., with a same age range. Thus, the first representative reference vector can include measured (including statistically determined, e.g., averaged) methylation levels for the sets of CpG sites and/or genes for sample subjects with the pathology.

Additionally, the model can generate a second reference vector for healthy sample subjects of a same or similar age as the subject. Thus, predicting the classification of the presence of the pathology in the subject can include comparing the feature vector to each representative reference vector and determining which representative reference vector the feature vector is most similar to. In some examples, there can be any number of representative reference vectors corresponding to any number of pathologies.

Each chronological age or age range can have its own set of representative feature vectors. Thus, the model can be considered to be comprised of sub-models of difference ages, where a sub-model has a set of representative feature vectors for different classifications. In other implementations, the model can determine a distance relative to all other representative reference vectors for all ages at once, but a distance of such reference vectors for a different age can have a weight that scales that dimension, such that the distance to reference vectors for different ages is generally higher than the distance to reference vectors for the same age.

Additionally in some examples, determining the classification of the presence of the pathology can include predicting, using the model, a biological age of the subject, comparing the biological age to the known chronological age, and determining the classification of the presence of the pathology in the subject based on the comparison. For example, to determine the classification based on the comparison, the method 1500 can include determining whether a difference between the biological age and the known chronological age exceeds a threshold. The threshold can be based on the known chronological age of the subject. If the difference exceeds the threshold, the classification may indicate the subject has the pathology (e.g., glioma). The difference can be positive (age acceleration) or negative (age deceleration), each corresponding to a type of age aberration.

Determining the classification of the presence of the pathology can also include predicting, using the age-dependent machine learning model, a biological age of the subject. The method 1500 may then involve comparing the biological age to a known chronological age of the subject, and determining the classification of the presence of the pathology in the subject based on the comparison, type, and wherein the pathology is for the particular tissue type.

### VI. AGE ACCURACY FOR DIFFERENT NUMBERS OF FEATURES

An accuracy of age predictions provided by a model can be determined for differing numbers of features. In one example, the differing numbers of features can be a number of genes. In another example, the differing numbers of features can be a number of CpG sites.

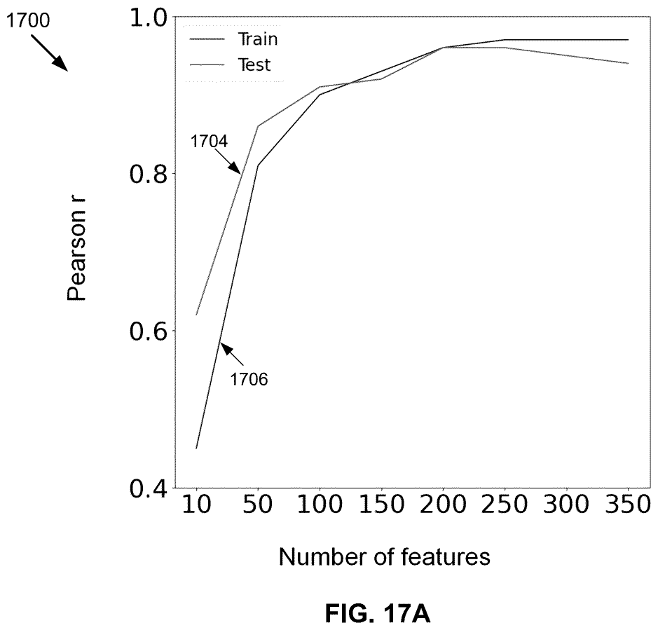

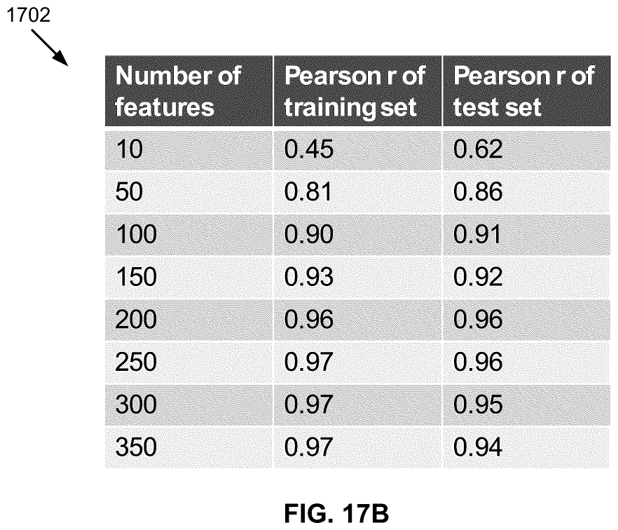

FIG. 17A shows a plot 1700 of Pearson's correlation coefficients between true chronological ages and predicted ages in the training datasets and testing datasets associated with the 785 subjects for which cfDNA 5hmc data was obtained and described above with respect to FIG. 3. The Pearson's correlation coefficients are each plotted against a number of features used in a corresponding age prediction model (e.g., the linear regression model described above) to predict age. Additionally, FIG. 17B shows a table 1702 of the Pearson's correlation coefficients and the numbers of features from the plot 1700.

In plot 1700, the numbers of features are numbers of genes used by the age prediction model to predict age. The genes used in the age prediction models associated with the Pearson's correlation coefficients in plot 1700 were selected from the 3,000 genes with highest normalized standard deviation values as shown with solid black dots in FIG. 2. Any number of genes (e.g., at least, 20, 30, 40, 50, 70, 80, 90, 100, 150, 250, etc. genes) may be used in an age prediction model to predict age based on cytosine methylation levels of the gene, in other examples.

Moreover, in plot 1700, line 1704 shows a trajectory of the Pearson's correlation coefficients associated with age predictions for the training dataset against a corresponding number of genes. In contrast, line 1706 shows a trajectory of the Pearson's correlation coefficients associated with age predictions for the testing dataset against a corresponding number of genes. In one example, as shown in plot 1700 and table 1702, when cytosine methylation levels of 100 genes were input to the age prediction model, the Pearson's correlation coefficients were 0.9 and 0.91 for the training and testing data sets respectively.

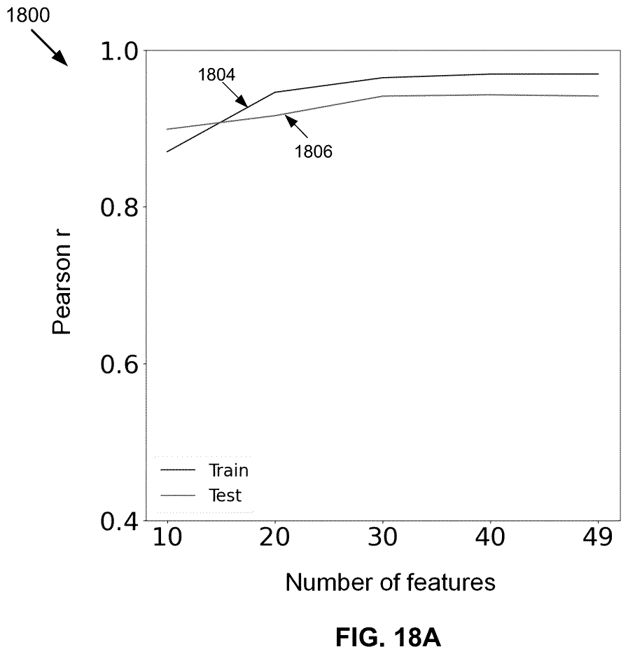

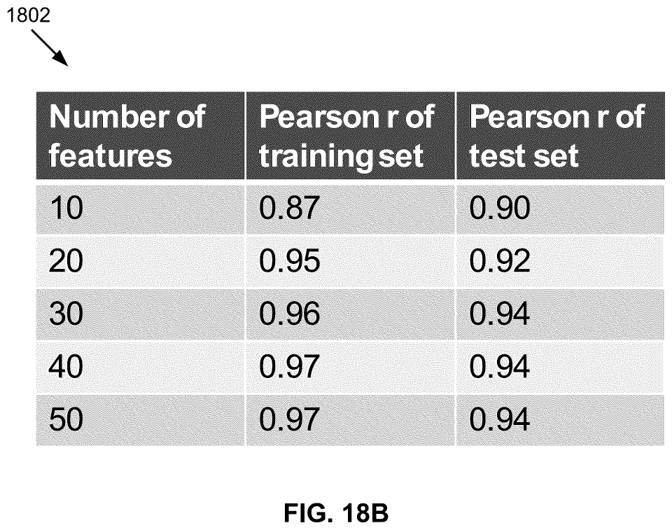

FIG. 18A also shows a plot 1800 of Pearson's correlation coefficients between true chronological ages and predicted ages in the training datasets and testing datasets associated with the 136 subjects. The Pearson's correlation coefficients are each plotted against a number of features used in a corresponding age prediction model (e.g., the linear regression model described above). FIG. 18B shows a table 1802 of the Pearson's correlation coefficients and the number of features from the plot 1800.

In contrast to plot 1700, the numbers of features in plot 1800 are a number of CpGs used by corresponding age prediction models in the age prediction. Thus, line 1804 shows a trajectory of the Pearson's correlation coefficients for age predictions for the training dataset against corresponding numbers of CpGs. Line 1806 then shows a trajectory of the Pearson's correlation coefficients for predicted ages for the testing dataset against corresponding numbers of CpGs. In one example, as shown in plot 1800 and table 1802, when cytosine methylation levels of 30 CpGs were input to the age prediction model, the Pearson's correlation coefficients were 0.96 and 0.94 for the training and testing data sets respectively.

The CpGs used in the age prediction models associated with plot 1800 were selected from the 49 CpGs described above with respect to the aging clock based on cytosine methylation levels. Any number of CpGs (e.g., at least, 5, 10, 20, 30, 40, 49, 50, etc. CpGs) may be used in an age prediction model to predict age based on cytosine methylation levels of the CpGs.

### VII. Example Systems

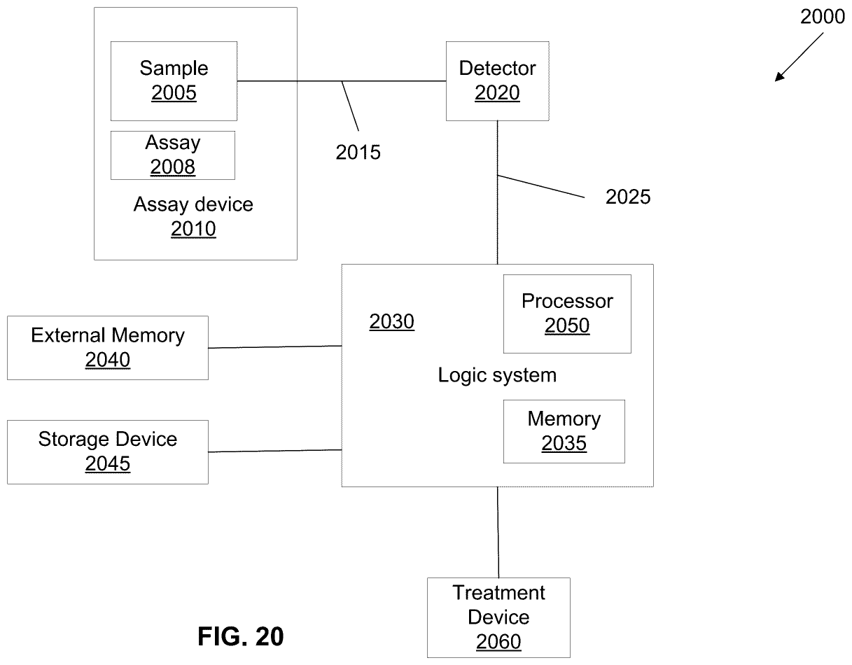

FIG. 20 illustrates a measurement system 2000 according to an embodiment of the present disclosure. The system as shown includes a sample 2005, such as cell-free nucleic acid molecules (e.g., DNA and/or RNA) within an assay device 2010, where an assay 2008 can be performed on sample 2005. For example, sample 2005 can be contacted with reagents of assay 2008 to provide a signal (e.g., an intensity signal) of a physical characteristic 2015 (e.g., sequence information of a cell-free nucleic acid molecule). An example of an assay device can be a flow cell that includes probes and/or primers of an assay or a tube through which a droplet moves (with the droplet including the assay). Physical characteristic 2016 (e.g., a fluorescence intensity, a voltage, or a current), from the sample is detected by detector 2020. Detector 2020 can take a measurement at intervals (e.g., periodic intervals) to obtain data points that make up a data signal. In one embodiment, an analog-to-digital converter converts an analog signal from the detector into digital form at a plurality of times.

Assay device 2010 and detector 2020 can form an assay system, e.g., a PCR system or a sequencing system that performs sequencing according to embodiments described herein. A data signal 2025 is sent from detector 2020 to logic system 2030. As an example, data signal 2025 can be used to determine sequences and/or locations in a reference genome of nucleic acid molecules (e.g., DNA and/or RNA). Data signal 2025 can include various measurements made at a same time, e.g., different colors of fluorescent dyes or different electrical signals for different molecule of sample 2005, and thus data signal 2025 can correspond to multiple signals. Data signal 2025 may be stored in a local memory 2035, an external memory 2040, or a storage device 2045. The assay system can be comprised of multiple assay devices and detectors.

Logic system 2030 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2030 and the other components may be part of a stand-alone or network connected computer system, or they may be directly attached to or incorporated in a device (e.g., a sequencing device) that includes detector 2020 and/or assay device 2010. Logic system 2030 may also include software that executes in a processor 2050. Logic system 2030 may include a computer readable medium storing instructions for controlling measurement system 2000 to perform any of the methods described herein. For example, logic system 2030 can provide commands to a system that includes assay device 2010 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 2000 may also include a treatment device 2060, which can provide a treatment to the subject. Treatment device 2060 can determine a treatment and/or be used to perform a treatment. Examples of such treatment can include surgery, radiation therapy, chemotherapy, immunotherapy, targeted therapy, hormone therapy, and stem cell transplant. Logic system 2030 may be connected to treatment device 2060, e.g., to provide results of a method described herein. The treatment device may receive inputs from other devices, such as an imaging device and user inputs (e.g., to control the treatment, such as controls over a robotic system).

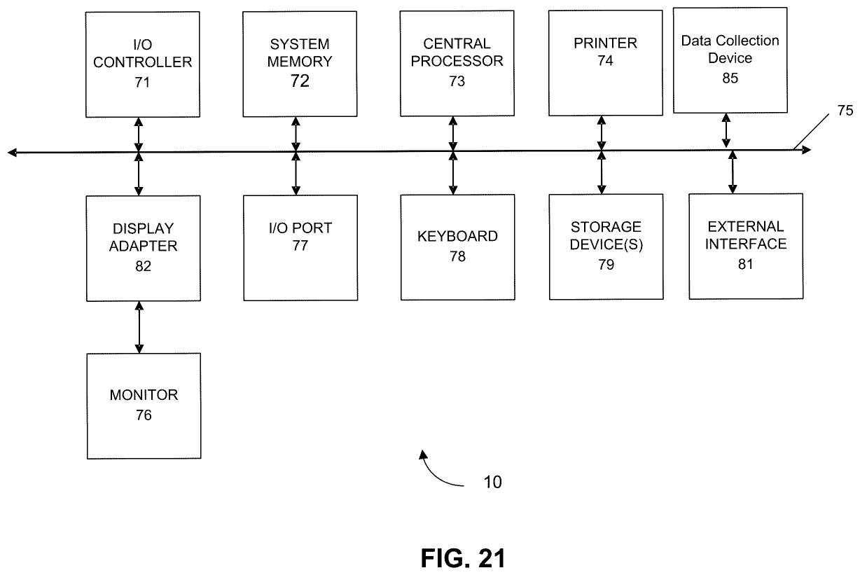

Any of the computer systems mentioned herein may utilize any suitable number of subsystems. Examples of such subsystems are shown in FIG. 21 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 21 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

### VIII. Tables

The genes for the eight clusters (groups) in the examples above are provided in Table 1 below. Various embodiments can use all or fewer numbers of genes for any given cluster.

TABLE 1
Genes corresponding to the 8 clusters from FIGS. 8A-8D.
Cluster 1

| | | | | | |
|---|---|---|---|---|---|
| TMCO5A | CLDN1 | HS3ST3B1 | ADGRL2 | TGFBR3 | RYR3 |
| PLCXD3 | KIAA1211 | ROBO1 | AXDND1 | BANK1 | B3GNT9 |
| CPEB1 | DNAH5 | TEX36 | FAM19A2 | PTGER3 | CEP135 |
| CHRM3 | COL24A1 | TAS1R3 | PRKG2 | LRIG1 | NOX4 |
| TTC27 | NRBP2 | FASN | GCSAML | ASPDH | GNG11 |
| PAPPA2 | ADGB | BSPH1 | AXIN2 | CBLN4 | ATP8A1 |
| ZNF521 | ZNF726 | EFCAB3 | NAA11 | PLAG1 | FRAS1 |
| MORC1 | TNIK | SSBP2 | FAM83H | GNAT1 | RFNG |
| LRP12 | GTDC1 | MMRN1 | COL25A1 | IL33 | ANKS1B |
| KHDRBS2 | CLVS2 | KIF18A | ZNF431 | MAP2 | PKIA |
| PLCL1 | CEP112 | ZFPM2 | ADAM23 | RTN1 | HCN1 |
| DGCR6 | RNF17 | PKHD1L1 | RTKN2 | CDH9 | PRR27 |
| ARHGEF16 | COL18A1 | RAC3 | NKAIN2 | GUCY1A2 | ARHGAP28 |
| NEGR1 | SLC9C1 | LRRC45 | CHODL | TINAG | RP11-432B6.3 |
| DKK1 | SLIT2 | KIF20B | ZNF682 | MEIS1 | MEGF10 |
| PTPRR | HMGCLL1 | SMOC2 | NBPF1 | RGS5 | KCNK2 |
| FGFR3 | RIT2 | ADAMTS12 | SARDH | GPS1 | GPR27 |
| ZNF626 | PHF21B | FAM150A | SNRPN | SLC13A1 | PRKD1 |
| ROBO2 | TEX9 | MAP2K7 | GPC5 | FSIP2 | DYNC2H1 |
| C1orf94 | GRM8 | TH | CATSPERB | NRCAM | MOBP |
| FGFRL1 | FHOD3 | NBPF12 | SLC24A2 | SEMA3C | AMPH |
| STK33 | ZNF382 | ZNF568 | MYRF | MYEF2 | RSPO2 |
| PDZRN3 | CASKIN1 | ZNF679 | ADGRF4 | GYPB | CAMK4 |
| FAM27A | BMPR1B | THEMIS | AGXT | NPFFR2 | ZNF714 |
| TMEM240 | KCNU1 | SYCP2 | SPAG17 | DIRC1 | ZNF540 |
| PQLC2L | FAM19A1 | DCDC2 | KIAA1324L | GLI4 | DSCAM |
| SLC25A22 | MAPK10 | CNBD1 | ZFP30 | LRRC3B | MKRN3 |
| PPP1R9A | KCNH8 | FOXA1 | DUXA | MYH2 | ZNF573 |
| PDE1A | HOXB13 | DEC1 | FAT3 | RGS7 | CADPS |
| LRRTM4 | ADGRF2 | NPAP1 | IMPG1 | NPTX2 | NPSR1 |
| PPFIA2 | JPH1 | UGT2B28 | ZNF66 | ZNF208 | FLG2 |
| ADCY8 | KCNC2 | ADAM29 | HFM1 | ZNF729 | MROH9 |
| RP11-215A19.2 | ASTN1 | SYT10 | ZNF404 | C16orf78 | CALCR |
| GRM5 | LAMA5 | CCT8L2 | CTNNA2 | KLHL17 | TMEM132D |
| ZNF420 | ZNF99 | TRPC3 | KCNE4 | AC010760.1 | CDH20 |
| IRX2 | TAS2R1 | VWC2L | C4orf22 | THSD7B | C12orf50 |
| USP29 | CBLN2 | SLC17A6 | SLC35F1 | PLCH2 | SLC25A21 |
| AGBL4 | GRXCR1 | PAX7 | ZNF536 | H1FX | ZNF676 |
| ZNF708 | SYNPR | GAB4 | GRM6 | ATAD3B | TPTE |
| CH17-140K24.8 | EBF2 | SPAM1 | ZNF479 | KCNH5 | PAX2 |
| UGT2B15 | SLC5A7 | DNAH7 | SYCP1 | NELL2 | FAM86B1 |
| NMU | ZIM3 | ADAM18 | NBEA | CA10 | AC092835.2 |
| ADAM2 | UNC13C | SHISA6 | PTPN20B | NXPE4 | CA8 |
| KCNV1 | FGF10 | NKAIN3 | MCHR2 | ALX1 | TRIM49B |
| SOGA3 | PTH2R | LAMA1 | NLRP5 | KCND2 | TFAP2D |
| GPRC6A | ABCA12 | NPY2R | DHX16 | ADGRB3 | ANKRD45 |
| OR4C46 | TMEM184A | NPIPB7 | RIMS2 | CBARP | SLC27A6 |
| TCEA2 | ZNF610 | ZNF667 | WSCD2 | HS6ST3 | CADM2 |
| MUC5B | PSG7 | OR4N2 | TSPAN19 | RP11-577H5.5 | ANKRD30BL |
| SPAG11A | UNC80 | ASPSCR1 | SLC35F4 | ZNF732 | MYH1 |
| SLC35F3 | NGF | PRB1 | SMIM17 | RIMS1 | HHIP |
| ZNF716 | GRIN2A | MYOZ2 | CCDC39 | CEBPB | MUC2 |
| ANKRD30B | CDH8 | SKOR2 | DEFB116 | HTN1 | FMN2 |
| KLRC1 | FHL5 | WFDC13 | GPR139 | GLRA3 | TMPRSS11F |
| CALCRL | KCTD8 | HTR1A | SLC5A8 | KCNIP4 | ZNF454 |
| ZNF560 | ZPBP | GPR158 | ZNF83 | C21orf59 | RIC3 |
| NXPE2 | BARX2 | C14orf177 | HTN3 | C14orf39 | LRRIQ3 |
| SYT14 | TMPRSS15 | OR6C2 | TTC34 | MUC22 | TRAT1 |
| NDST4 | SNAP91 | EDIL3 | PNLIP | DEFB4A | CSRNP3 |
| B3GALT5 | COL6A1 | B3GAT2 | MYH8 | ZSCAN1 | PRIM2 |
| CSMD3 | FAM171B | VGLL3 | PCSK2 | MYL1 | GNGT1 |
| ADAD1 | GABRG2 | MYO3A | AC017081.1 | TTC29 | PRODH |
| CDS1 | USP17L7 | TMEM26 | CDH18 | GRIA2 | CLDN3 |
| KRTAP6-1 | GRID2 | TMPRSS11E | RRP7A | SLC4A10 | SORCS3 |
| KLHL14 | GLIPR1L2 | STXBP5L | HS3ST4 | IQUB | EPHA5 |
| TPRXL | TWIST1 | NXPE1 | PCP4 | OR4C11 | PPP1R3A |
| MUC16 | FAM19A4 | ZNF578 | LHFPL3 | LCE3C | CCHCR1 |
| ZNF492 | PTPRZ1 | VSTM2B | NMUR2 | WSCD1 | HIST1H1E |

Cluster 2

| | | | | | |
|---|---|---|---|---|---|
| EVC | ESRRG | EPB41L4A | ADAMTS9 | EVC2 | SULF1 |
| PRKCB | MRVI1 | PRKCQ | CACNA1C | SYK | PRR5L |
| PHACTR3 | SLC24A3 | CELF2 | MCTP1 | GRIK3 | UBASH3B |
| ANO2 | GRAP2 | F13A1 | SPSB4 | PIK3R5 | DGKG |
| IGF2R | TMEM163 | HPSE | IKZF1 | EHD3 | PVALB |
| VCAN | TMPRSS13 | MYH6 | ANK1 | ARHGEF3 | TRIB2 |
| RSU1 | APBA2 | KAZN | KIRREL | CD6 | APCDD1 |
| FGF6 | TLDC1 | ARHGAP15 | FYB | SPTA1 | HACD4 |
| APCDD1L | ITPKB | SEC14L5 | TSPAN11 | C1orf95 | ABCC1 |
| CLEC2L | CLEC12A | MYT1L | CRISPLD1 | ASAP2 | GP6 |
| VWF | CLEC1B | KCNQ5 | RAB6B | CHST15 | MDS2 |
| CD274 | STX11 | THUMPD2 | THBS2 | UBASH3A | PARVB |
| PLPP7 | LPAR5 | PRDM5 | RGS10 | SLC37A1 | CYTH4 |
| SLC7A1 | LDB2 | ZEB2 | GAS6 | WNT11 | RGS18 |
| RXRA | IGSF23 | OLFML2A | ITGA2B | KIF19 | ORM1 |
| TSPAN15 | GIMAP5 | ADAM22 | VASH1 | PARVG | COTL1 |
| MEF2C | CMKLR1 | FLI1 | GNG2 | CLC | COL6A5 |
| OSBPL3 | SERPINA1 | NCF4 | GPR20 | ADARB2 | SLC2A3 |
| NADK | FAR2 | GPR63 | HS3ST3A1 | MGAT3 | DUSP22 |
| KCNK13 | LCP2 | PADI4 | P2RX1 | CD47 | PTGS1 |
| CATSPER1 | SMAD7 | CSF2RB | SIPA1L2 | CPXM1 | ATP2A3 |
| BPIFB3 | SKOR1 | PTPRC | RUNX1T1 | ADGRE3 | HMX3 |
| NEDD9 | RASGRP2 | SRC | CD81 | GSDMA | CPXM2 |
| A4GALT | SAMSN1 | SLITRK5 | CTTN | TESC | MFSD2B |
| PEAR1 | IQCE | CD33 | CD53 | DENND3 | ARHGEF15 |
| REM1 | NKX1-1 | TGFB1 | RAMP3 | JAK3 | OPRM1 |
| NCR1 | DOK2 | MS4A4E | FOXD4L1 | ALG1L2 | ALAS1 |
| ABHD11 | AXIN1 | RASSF2 | ABR | NDNF | SPATA16 |
| ADCY6 | GPR35 | NUDT1 | MMP9 | PDLIM7 | UGCG |
| ZNF784 | TFEB | EMB | C2orf57 | SNTN | NRGN |
| SIGLEC9 | SGK223 | FAM90A1 | LGALS2 | PTPN14 | SIRPD |
| DISC1 | TNFSF10 | ADGRL4 | TNNT1 | FAM105A | OSBPL5 |
| MYL9 | CNTNAP3B | NPY1R | GABRD | SCN2A | ATP10B |
| CHSY3 | STK32C | NPIPB15 | OPN5 | TNFAIP2 | CYYR1 |
| RASA3 | IGSF11 | UNC5C | NTSR1 | MS4A6A | SEL1L2 |
| PLAC8 | FREM1 | TLR2 | GLIPR2 | ANKRD36 | APOBEC3A |
| TMTC2 | OR2W3 | GPR12 | ICAM5 | RAC2 | TNFRSF1B |
| DBN1 | NTNG2 | OR2T5 | FAP | RP11-152F13.10 | PIK3CD |
| B4GALT5 | MS4A3 | CNTNAP3 | AF165138.7 | CD200R1 | AL591479.1 |
| TPD52L2 | RHBDF2 | PTPRE | MSRB1 | GPR132 | SCN7A |
| LILRB4 | AP5B1 | PRSS27 | C1orf106 | HIST1H4L | CD300LB |
| CHRFAM7A | EMILIN1 | CD1B | KIR2DL3 | GRAMD4 | C15orf52 |
| FAM78A | TCP11L2 | GULP1 | SPIC | CLEC18B | STK32B |
| AZU1 | RP11-195B21.3 | RP11-294C11.3 | RELT | CERS3 | AL627309.1 |
| LGALS9B | GJB7 | NTRK3 | ZIC1 | BTNL3 | CACNA1E |
| FGR | TRPC4 | MMP25 | SEMA3A | TRIM27 | CD37 |
| LVRN | SLC39A12 | TMEM38A | CLEC4D | BTNL8 | MRGPRX1 |
| SPI1 | HOXD11 | KIR2DL1 | STK32A | HOXC4 | SERPINB10 |
| TMC8 | FERD3L | LAIR1 | PRR16 | PGLYRP1 | LPCAT2 |
| MARCKS | NPIPB6 | LRRC25 | PCDH17 | CALML5 | PI3 |
| ADCY4 | KCNK18 | DMBT1 | TNXB | TMEM52B | CPLX2 |
| KBTBD11 | CPPED1 | FAM198B | ZNF469 | C1QTNF8 | AGTPBP1 |
| SNTG2 | LCN2 | KIR3DL1 | PADI2 | TDRD5 | NBEAL2 |
| KCNJ12 | LILRA1 | POTEG | HCAR2 | SFRP1 | DIO2 |
| NANOG | TLR4 | PITPNM1 | PIWIL4 | TYROBP | AMH |
| GPR42 | SMN2 | FPR1 | RGS17 | FCGR3A | LRRC7 |
| HCK | KRT12 | ADRA2A | MMP27 | UNC5A | IGSF9B |
| NFAM1 | PREX2 | NEUROG3 | GFI1 | KCNAB2 | ARRDC5 |
| CARD9 | RCSD1 | DNER | LILRB1 | ANKRD18A | ENTPD1 |
| ZNF365 | NOTCH4 | KLF2 | ABCA7 | FXYD5 | ABI3 |
| SLC35E2 | KCNA3 | HRH2 | NPIPA7 | FAM151B | TRIM26 |
| CCR2 | TSHZ3 | CBWD5 | TRPM2 | TBC1D3C | TMEM55A |
| CLEC6A | STEAP4 | TPBG | NPIPB9 | TNFRSF14 | MX2 |
| HSPB8 | CCZ1 | AADACL3 | ANKRD20A1 | KRT23 | VSIG2 |
| IL32 | MARCO | RBM38 | HK3 | LAMA2 | RAB11FIP1 |
| PDGFD | MSR1 | EGR2 | AGAP2 | CILP2 | SLFN5 |
| FCAR | C10orf53 | TSPAN16 | SERPINB2 | LRRC37A2 | MYO1F |
| OR3A3 | ISYNA1 | CCDC129 | GALNT3 | GIMAP8 | PARP8 |
| FOLR3 | ADGRE5 | CD300E | VSTM1 | PSTPIP1 | CAMP |
| NLRP12 | ZNF215 | MS4A4A | ANKRD13D | RAB31 | ARL17A |
| IL22RA2 | NCLN | GBA | GAS7 | FMNL1 | LYZ |
| SYNGAP1 | RCVRN | ARC | TRIM49C | RSPH10B | PPP1R17 |
| CEBPE | ADRBK1 | RIMBP3C | CD93 | PLBD1 | KIR2DL4 |
| TARM1 | LSP1 | CTSG | RASEF | POTEM | RPE65 |
| WASH1 | POTED | PARM1 | MS4A13 | AC008132.13 | ITGB2 |
| GATA5 | NOMO3 | AL592284.1 | ST6GALNAC5 | S100A12 | GCA |
| GALNTL6 | LILRA5 | SLCO4C1 | TMTC1 | PCDHB9 | UPK3A |
| MLNR | ATP8B4 | LPL | GABRG1 | EFHD2 | TMC6 |
| TPPP3 | ZNF595 | AHRR | PCDHB8 | CBWD3 | S100A11 |
| NAIP | MNDA | SGCZ | PDE1C | RNF166 | PSORS1C1 |
| SLC36A4 | HIST1H2BO | ADAM8 | CSGALNACT1 | RFPL4AL1 | BPI |
| GABRA4 | TMEM236.dup | RNASE3 | GALNS | CCDC107 | AL353791.1 |
| RIMBP3 | CEACAM3 | NCAM1 | TTN | FAM27E1 | CEACAM4 |
| EFNA5 | FSHR | IVNS1ABP | REXO1L1 | KCTD12 | ZNF804B |
| KCNJ2 | MPEG1 | CLEC4G | SCIMP | DEF8 | TGFBR1 |
| CCDC182 | SLC28A3 | PRAM1 | MMP8 | FCN1 | ADAMTS18 |
| GIF | CDH7 | ZNF770 | HRH4 | PTCHD4 | TRIM64C |
| OR1D5 | TNNI2 | METRNL | LPCAT1 | LTF | SLC8A1 |
| NSUN5 | FCGR3B | HLA-B | KLHDC7B | SDR16C5 | MEFV |
| CELA3A | SGPP2 | LILRB2 | RASA4B | CD177 | KIAA1211L |
| RP11-514P8.6 | ATHL1 | S100A9 | | | |

Cluster 3

| | | | | | |
|---|---|---|---|---|---|
| CHST9 | FAT1 | ADH1B | PROX1 | ADH4 | SDC2 |
| APOB | TF | FMO3 | SALL1 | NUAK1 | CFH |
| GPAM | EXT1 | CNN3 | GATA4 | TBX3 | FARP1 |
| PAH | NR1H4 | ADH6 | TOX3 | SERPINA5 | RAI14 |
| MYO1B | CYP27A1 | ITIH2 | ELOVL2 | CDH2 | TEAD1 |
| GLIS3 | IGFBP1 | HAO2 | SLC38A4 | PRLR | BAIAP2L1 |
| KNG1 | SERPINA6 | ADH1A | APOH | SLC7A2 | IGF1 |
| MAGI1 | HSD17B2 | C6 | A1CF | ACSM2A | PLG |
| MAL2 | TTPA | DTX1 | SLC27A5 | CYP2C9 | LRIG3 |
| ALDOB | BAAT | FAM171A1 | SLC22A3 | CYP2C8 | PTGR1 |
| DOCK4 | KIF25 | CYP3A5 | PTPRK | NPAS2 | NINL |
| TTLL2 | TECTB | RHPN2 | HABP2 | SPP2 | SATB2 |
| CFHR3 | PPARGC1A | SLC7A13 | MROH2B | ONECUT2 | GC |
| PLPPR1 | RP4-608O15.3 | NTN4 | ACACB | CPS1 | AGXT2 |
| SORBS2 | CXADR | PID1 | PTP4A1 | FRK | RND3 |
| ALDH8A1 | MLLT4 | ENPP1 | ADH1C | MET | ONECUT1 |
| TDO2 | TACC2 | SLC22A1 | SERPINA11 | GHR | MTUS1 |
| NHSL1 | TMPRSS2 | FN1 | MLIP | RP11-400G3.5 | ACSS3 |
| GLYATL3 | ACSM2B | TBX2 | SLC13A5 | PALMD | CYP4A11 |
| LIPG | GPR39 | SULT2A1 | C7 | PKHD1 | GPC6 |
| RNF152 | SLC19A3 | TMEM176A | MVK | LARGE | MOCS1 |
| C8A | MTMR7 | UGT2B4 | ACSM5 | SLC6A1 | SERPINA12 |
| TBC1D12 | TRPM8 | FGL1 | HMGCS2 | BTBD16 | CYP1A2 |
| ADM | ABCG8 | MLXIPL | 45545 | SMIM14 | SUCLG2 |
| FBP1 | ADRA1A | CADM1 | RGMB | TJP1 | HNMT |
| KLKB1 | ISM1 | EPB41L4B | FMO4 | FGF14 | IRF6 |
| CNTN3 | C8B | C2orf72 | RIPK4 | LYPD1 | PPFIBP2 |
| HRG | RP11-766F14.2 | MCC | HNF4G | FAM110C | F13B |
| GREM2 | C2CD4D | ARHGEF10L | SHFM1 | C4orf19 | PLCE1 |
| COL6A2 | CYP3A4 | GDA | ADAMTS17 | CYP2B6 | LIN7A |
| THSD4 | RBFOX2 | FMO2 | PON3 | DDC | PCDH1 |
| CTSO | FTCD | ALDH2 | RORA | NIPSNAP1 | HSD3B2 |
| SRD5A2 | CPB2 | ACOT12 | SHH | LMX1A | PNPLA3 |
| ACMSD | CUX2 | AGMO | FGFR2 | CYP4F12 | TBX15 |
| UNC93A | MYH14 | KHDRBS3 | FBXO17 | FOLH1 | DIP2C |
| ZFHX4 | BBOX1 | DACT2 | FREM3 | SEC14L4 | TRABD2B |
| SCGB2B2 | GMNC | PRDM9 | SOX5 | PTPN3 | ABCC9 |
| HUNK | TMEM117 | HAGH | FAHD2A | C14orf105 | FERMT2 |
| MIPOL1 | VSNL1 | ASS1 | USP30 | KITLG | SLC22A9 |
| GYPE | CREB3L3 | LYPD6 | TPRG1 | COLCA2 | GLDC |
| RNF180 | BCHE | LGI1 | SAMD5 | NEK10 | ETNPPL |
| ABCC11 | ZNF703 | TTC6 | MYO5B | ABAT | MTTP |
| PON1 | HAO1 | PTGFR | SNX7 | TMEM82 | C1orf53 |
| SLC15A1 | ARPP21 | SOX6 | NADK2 | CPQ | CTC-360G5.8 |
| ABCB4 | CCDC58 | ALDH7A1 | C9 | COBLL1 | CYP39A1 |
| CDX2 | NTN1 | SMOC1 | SCP2 | SLC22A2 | KCNJ8 |
| FETUB | MRTO4 | C5 | F7 | MUT | BMP4 |
| EMP2 | AFM | TMEM186 | PDLIM3 | CES1 | CDH1 |
| F11 | UGT3A1 | L1TD1 | KCNH7 | TBC1D4 | KIF21A |
| UBXN10 | ODAM | PDE5A | RALGPS2 | C11orf86 | CYP4F11 |
| SLC22A10 | CYP4A22 | RP11-545J16.1 | COL5A2 | GCKR | GAS2 |
| WNT5A | RGPD3 | XKR9 | ADH7 | CFHR5 | ESR1 |
| PDILT | SP5 | SH3D19 | MED23 | TMEM192 | TMEFF2 |
| CDON | RGS7BP | SHROOM3 | WIF1 | SLC41A2 | NDFIP2 |
| PRKACB | RP11-125O5.2 | SHC2 | LCA5 | COL27A1 | LAD1 |
| PVRL3 | PIK3C3 | SCN8A | COBL | IFT57 | KCNJ3 |
| PLA2G4C | ELFN1 | MGST1 | XDH | DSG4 | LGR4 |
| CNTN4 | C1orf168 | EPB41L5 | CFI | COL4A3 | FAM149A |
| SH3GL3 | MMAB | IQCJ-SCHIP1 | MOCOS | APBA1 | AVP |
| ALDH1L1 | IYD | SPTLC3 | PTPRG | F10 | AC137056.1 |
| TMEM200A | SLC15A5 | OGDHL | THRB | PIWIL1 | GDPD4 |
| ENTPD8 | TIMM44 | IFRD2 | BET1 | GPHN | PAPPA |
| VMO1 | MEI4 | PRDM6 | DGKB | CSN3 | EYA4 |
| PTN | SNCAIP | KMO | MACROD2 | DRD4 | PGBD5 |
| MICU3 | PSAT1 | MASP2 | ADGRA3 | SLC45A2 | ZNF385B |
| SYT7 | DYNC1I1 | DNAH11 | LPA | CROT | DSG1 |
| NDC80 | GGH | NWD2 | NAT2 | GRIN2B | SLCO1B1 |
| GAREM | PRKAA2 | NRAP | NOL4 | SLC5A12 | AFP |
| MOGAT1 | ABCA8 | SRCIN1 | CXCL13 | TMEM178B | ASIC5 |
| HSD17B3 | METTL15 | RP5-862P8.2 | KCND3 | PRKG1 | C1orf185 |
| NR3C2 | WDR64 | ASAH2B | CYP7B1 | NUDT7 | RANBP3L |
| THOP1 | NUBPL | AMY2B | IGF2 | PTPRT | ZNF385D |
| PACRG | RDH8 | POMZP3 | PALM2 | GFRA1 | SNTG1 |
| CYP4V2 | ERICH3 | TMPRSS6 | ZNF324 | UGT2A1 | CD36 |
| MPV17L2 | MROH2A | LRP1B | ALCAM | ANO1 | DLG2 |
| GOLT1B | WDR97 | ADRA1B | LMO3 | SLC17A1 | HNRNPCL1 |
| HIBADH | GRB14 | C1orf87 | CYP4X1 | PLOD2 | POT1 |
| SLC7A9 | SLC17A4 | ASB3 | HEPHL1 | CDK1 | GCDH |
| WDR17 | FER | LGSN | SLC3A1 | SHTN1 | PRH1 |
| EXTL2 | LRRC66 | TP63 | CNTN5 | CCDC14 | CLSTN2 |
| SFTA3 | ADGRG6 | PSG4 | MAP9 | LECT2 | KCNIP3 |
| UBE2E3 | AASS | FBXO27 | SPACA7 | FOXP2 | PLPPR4 |
| TENM4 | PSG5 | STPG2 | DNAH14 | ISL1 | ALDH1A1 |
| ARHGAP20 | CELF4 | MPDZ | SLCO1A2 | ATP8A2 | SLC4A4 |
| LRAT | NDST3 | MUSK | CCDC102B | MAGI2 | CNTNAP2 |
| NR2F2 | TYRP1 | APOA4 | DOCK3 | RUNDC3B | CYP4Z1 |
| ANK3 | ISX | ADAM7 | LGR5 | HPGD | NOS1 |
| IMPG2 | PLA2G2C | MMP26 | COL22A1 | HIST1H2BG | SPATA31A6 |
| LUZP2 | C12orf42 | CPED1 | EPGN | KCNH1 | SGCD |
| CNTNAP4 | PPP2R2B | AL162431.1 | FAM160A1 | METTL11B | FLG |
| SMCP | CLVS1 | ZNF248 | HLA-DQB1 | GRID1 | FAM134B |
| FRZB | LRRC9 | C12orf40 | NLRP14 | TSPAN8 | POU6F2 |
| ELAVL4 | HLA-DRB1 | SPAG11B | ASCC3 | OR51E2 | ISPD |
| ERC2 | SPACA1 | CES5A | CCDC190 | SCN9A | COL6A6 |
| DRD1 | MRAP2 | CEP290 | TRIML2 | MUC17 | AKAP6 |
| MTUS2 | C14orf144 | LURAP1L | SCN3A | NYAP2 | ADAMTS6 |
| GLT6D1 | SPON1 | NLRP8 | WFDC8 | WDR72 | ABCA5 |
| SH3GL2 | HLA-DRB5 | C10orf67 | ASPM | NMS | ST18 |
| ANKRD20A4 | OR5M8 | DEFB114 | IKZF2 | CEACAM20 | SYT9 |
| ZMAT4 | ZDHHC11B | POTEC | FGF12 | HLA-DQA1 | CEBPD |
| DAAM2 | NAALADL2 | LEKR1 | GP2 | PLPPR5 | DCHS2 |
| PIK3C2G | TPSG1 | GLYATL2 | ANKRD20A2 | NPB | DAB1 |
| GRM7 | FAM159B | APELA | USP17L15 | C8orf89 | KCNN2 |
| PLCZ1 | KRTAP4-9 | CDH13 | LRIT3 | IER5L | KLRC2 |
| PPP1R1C | ACVR1C | CTNNA3 | UBE2Q2L | RP11-219A15.1 | AJAP1 |
| ZNF429 | KCNA4 | SLCO1C1 | KIAA1024L | CCDC172 | ZBBX |
| OR5B12 | DDX60 | AMER3 | CEP126 | ASTN2 | GOLGA6B |
| ERVV-2 | TEX26 | TBX20 | CACNA1H | TMEM132B | SHANK2 |
| KIAA0825 | | | | | |

Cluster 4

| | | | | | |
|---|---|---|---|---|---|
| MEIS2 | FREM2 | PTPRM | SMCO4 | IDO2 | FEZF1 |
| SYT17 | CCDC170 | ARHGAP42 | LGR6 | COL5A3 | ARHGAP44 |
| OAS1 | PITX2 | CCDC82 | WASF1 | COL6A3 | TMEM196 |
| VLDLR | NTM | EPM2A | GCNT4 | RP11-514P8.7 | CACNA2D3 |
| DGKI | NUTM2E | PTPN20A | ASXL3 | NXPH2 | NCAM2 |
| LCE2A | MRC1 | ASCL1 | CDH10 | ABCC12 | OR10H3 |
| PRAMEF11 | ASB5 | CDH17 | GRM3 | FGF2 | C10orf90 |
| HTR5A | LGALS7 | | | | |

Cluster 5

| | | | | | |
|---|---|---|---|---|---|
| PIP5K1B | VAV3 | TMEM120A | BOK | SLC2A4RG | PTGDR |
| ASB6 | ZBTB47 | SERPINI1 | DACH1 | PRSS50 | SLC35D3 |
| RHOJ | KDR | AGRN | LIFR | TUSC3 | REXO1 |
| PFKL | VWDE | MMP16 | CIRBP | SEPT14 | NPIPA5 |
| GPT | SERTM1 | ANXA1 | SLC9A3R2 | ANO3 | PLPPR3 |
| GPX4 | TRPM5 | ZNF735 | PNPLA2 | TRIM49 | GPR78 |
| PCDH18 | IGSF5 | OPRK1 | ZNF658 | C11orf87 | GRIA4 |
| B4GALNT4 | PIP5K1C | ZNF43 | RIMBP3B | BAHCC1 | IFI44L |
| ZNF705B | | | | | |

Cluster 6

| | | | | | |
|---|---|---|---|---|---|
| FAM20A | APBB2 | CYP2E1 | C7orf72 | KCNT2 | NKX3-2 |
| TLX1 | GPATCH2 | STARD13 | ADCY1 | CPE | TFAP2A |
| SAMD12 | OSTN | NLRP11 | SLC1A6 | HPSE2 | FAM78B |
| B3GAT1 | TMEM8C | SAP25 | CYP2C19 | SYCE1 | RGSL1 |
| STEAP2 | PER1 | DTX2 | PCDH9 | FAM84A | ST8SIA3 |
| MAPK4 | SLC6A13 | NEBL | ADCY2 | CFAP69 | POTEB2 |
| EIF3C | BTBD11 | TENM2 | IFNA14 | HGH1 | TNC |
| C8orf34 | ME1 | CR769776.1 | RIMBP2 | GSTM2 | EPB41L4A-AS2 |
| HMCN1 | FAM27E2 | | | | |

Cluster 7

| | | | | | |
|---|---|---|---|---|---|
| POR | MBOAT2 | WFDC1 | XKR3 | DEPDC1 | HGS |
| HIC1 | MYO18B | P4HB | RECQL4 | GDF2 | TNNT3 |
| MLC1 | ABO | CEP128 | HHLA1 | RNF126 | CACNG3 |
| MRC1L1 | TRAF7 | MARCH1 | SSTR4 | TWIST2 | COL4A1 |
| KCNT1 | ING5 | SLC22A12 | RASSF9 | SCN1A | ARHGDIA |
| SBNO2 | GSDMD | ZNF628 | KLF16 | TCIRG1 | FSTL3 |
| IRF2BPL | ADAM32 | VAT1L | IL18RAP | ELAVL2 | FERMT1 |
| PXDN | CEACAM7 | AC138969.4 | ZNF486 | CSN1S1 | ZHX1 |
| MEGF6 | FAM132A | CORO1B | MMP17 | LY6E | OR6C6 |
| IRF7 | PCDHB1 | MAFG | TYMP | ZBTB7A | DOT1L |
| PIEZO1 | KRT222 | PWWP2B | TOR4A | C17orf62 | TRIM48 |
| SLC39A4 | MGAT4B | MICALL2 | MED16 | POLR2E | NOMO2 |
| CSNK1G2 | OR5P2 | EPPK1 | | | |

Cluster 8

| | | | | | |
|---|---|---|---|---|---|
| GABRB3 | ARMC4 | MPP7 | SPON2 | SAMD11 | GABRG3 |
| FAM83B | EREG | PARPBP | C5orf30 | SLC25A47 | OLFM3 |
| RBFOX3 | EFCAB1 | MKNK2 | CPNE4 | NOVA1 | ULK1 |
| NLGN1 | CAMK2N1 | ACAP3 | DVL1 | LRP5L | MMS22L |
| KCNQ2 | PIGQ | SYT16 | MFSD10 | CLUH | POLRMT |
| SLC9A3 | HYDIN | MRPL23 | C2orf54 | BSG | COL9A3 |
| CROCC | CTC-512J12.6 | TSPAN4 | ANHX | MUC6 | MIB2 |
| SOX11 | COL8A1 | USP17L2 | PLXNA1 | VWA1 | HMX1 |
| DAOA | ZFP42 | CDHR5 | KRTAP19-3 | PANX2 | HEXA |
| PLXNB2 | PCDH15 | KRTAP19-7 | NEU4 | HGFAC | PRB2 |
| ZNF117 | NSMF | TAC1 | ERBB4 | TBX5 | GRIN3B |
| PDE4D | OXER1 | INTS1 | CCDC178 | ABHD16B | TBC1D3 |
| BRINP3 | GPR26 | ZNF471 | PKP3 | PTBP1 | RBFOX1 |
| DEFB107A | NME3 | QRFPR | MC2R | KM-PA-2 | PDIA2 |
| HIST1H4D | IER2 | TERT | OR4C6 | LRRIQ1 | TMEM261 |
| KRTAP20-4 | NLRP13 | HTR1E | MUC5AC | PCDH10 | CNTN6 |
| YIPF7 | BAG6 | HMG20B | FAM50B | OR6C68 | ABCA2 |
| RNF208 | ZNF90 | GAMT | ZFPM1 | TRIM60 | HS3ST5 |

The genes used for the organ-specific ageing models are provided in table 2 below. Various embodiments can use all or fewer numbers of genes for any given cluster.

Bladder

| | | | | | |
|---|---|---|---|---|---|
| MATN2 | MXRA8 | NAP1L2 | LINC01345 | TSHZ3 | ITPR1 |
| SLC26A4-AS1 | MYADM | TIAM2 | CSRP2 | SLC25A4 | KLHL38 |
| WNT5B | C15orf41 | EGLN3 | MICAL1 | SMOC2 | PFKP |
| COL21A1 | AGBL5 | TMEM159 | ZNF443 | LINC01088 | C1orf216 |
| CHRM3 | CCDC136 | FN1 | TSLP | SCGB1A1 | TBX5 |
| FLNC | LINC01788 | MGC27382 | SLC30A8 | SAMD13 | SBSPON |
| CLCA4-AS1 | UBB | COL6A3 | LINC01160 | NGF-AS1 | GSN |
| LAMA4-AS1 | CHRM2 | EBF1 | WFDC1 | HEPH | KLHL21 |
| NRP2 | SMIM1 | ASPN | XCL2 | SVIL | SSC5D |
| IL17B | COX8A | WFS1 | ACKR3 | AKR1B10 | ADARB1 |
| AKR1B15 | MRGPRF | PRSS23 | LOC101928404 | FAM166B | CHRDL2 |
| NCS1 | LOC101928596 | HIF3A | FENDRR | ENTPD1 | LAMC1-AS1 |
| SORBS1 | GEM | HK1 | MMP12 | ACTN1 | CAMKK1 |
| TNS1 | LRFN5 | TXNRD1 | BDNF-AS | MIR29B2CHG | LOC148696 |
| RNU6ATAC35P | GLP2R | SGCA | LAMA4 | G6PD | CHRM3-AS2 |
| SCUBE3 | PRR26 | LINC00703 | CX3CL1 | LINC00710 | CALML3-AS1 |
| NENF | MAP1A | MAGEE1 | LINC01605 | AHNAK2 | PRUNE2 |
| CACNA1C | PKIG | P2RX1 | CAVIN2 | PBX1 | KCNMA1-AS1 |
| LINC00865 | LINC01375 | NMRK2 | CCKAR | RAMP1 | DHX35 |
| SAMMSON | TBX20 | CDH17 | PINLYP | POLD3 | LOC283038 |
| IL4 | NFKB2 | DCP1B | ST8SIA1 | SNORD131 | FSHR |
| MBOAT1 | L3HYPDH | SPON1 | JPH2 | CACNB2 | PRRT1 |
| GAGE1 | FLNB | KLF17 | NDUFA12 | GPRASP1 | RBM24 |
| FAM181A | SELENOW | SRPX | FAXC | MYOF | SLC2A4 |
| LOC338694 | PART1 | P4HA3-AS1 | MTARC1 | WTAPP1 | BCHE |
| LINC02764 | LOC101928847 | DDX53 | LINC02763 | ADGRD1 | PPDPFL |
| PLA2G5 | KCTD15 | ATP13A2 | LINC00942 | LINC00940 | TIMM44 |
| CACNA1C-AS4 | CACNA1C-AS1 | NIBAN1 | A2MP1 | RANBP3L | ADAM33 |
| LMO3 | TUBB6 | SPEG | HEYL | SCHIP1 | SLITRK3 |
| EMP3 | LINC02402 | PTGIS | SNAPC2 | PHYHIP | SHISAL1 |
| MYOCD | AXL | TBX4 | VCL | DHRS7C | CCDC190 |
| CCDC60 | MCAM | SLC8A1 | SSX5 | FYCO1 | LOC101927583 |
| IL26 | PTGFR | CEP295NL | GTSF1 | PID1 | SLC2A8 |
| RASL12 | EDN2 | PPP1R3B | LINC00173 | PRKCA-AS1 | KCNK7 |
| LINC00310 | VPS72 | LINC00367 | LINC00424 | ROR2 | CD44 |
| LINC00445 | LINC00332 | TGFB111 | TCF21 | SERP2 | LINC01198 |
| IL4R | PTGS1 | LINC00561 | ITGA7 | LINC00351 | LINC00454 |
| LINC00565 | LINC02332 | NIPAL4 | CIDEC | CDSN | PPP1R3A |
| LINC02310 | RUSC2 | IGSF9B | LTBR | PDLIM3 | NTNG1 |
| PPP1R3C | EGR1 | DPYSL3 | MSX2 | TNC | PNCK |
| CDC42EP3 | ACTC1 | LAMC1 | LOC101928174 | LOC145845 | SLITRK6 |
| CAMTA1-AS2 | HSPG2 | TNFAIP8L3 | TSPAN2 | LOC101929076 | PELI2 |
| SBF2-AS1 | MPP7 | RAB23 | ANGPTL2 | TWIST2 | HOXA5 |
| CTPS1 | LINC01290 | ADAMTSL3 | MIR1-1HG | LOC100288162 | SNX29P1 |
| KIZ | EPHA3 | NSMCE1-DT | COL4A6 | SNX29P2 | PLPP7 |
| LOC101928343 | MMP28 | AHNAK | KCNA5 | FBXO30 | S100A4 |
| HCCAT5 | DAPK3 | LINC02135 | LINC00917 | SPARCL1 | PLPP1 |
| NPAS4 | GATA5 | RAI1-AS1 | LINC01563 | STON1 | METTL7B |
| ITIH5 | ITGA8 | LINC00520 | LOC101927572 | LOC100288866 | LINC01969 |
| SHROOM3 | TMEM229A | CH25H | KCNK6 | LAMC2 | ECM2 |
| ADAM11 | CD9 | SEPTIN9-DT | LOC400622 | XG | LOC400627 |
| EHBP1L1 | DLGAP1-AS1 | LINC01387 | LOC105372028 | BLID | YBX2 |
| LHCGR | HSD17B6 | PKD1 | HIGD1B | ABCC1 | TMOD4 |
| COL12A1 | PTHLH | ARHGAP23 | PMP22 | KANK2 | PTGER1 |
| TMEM35A | PRSS48 | HSPB8 | FHL3 | TSPAN15 | DM1-AS |
| TACR2 | KCNMB1 | ZNF702P | SEMA3C | GPR158 | DCHS2 |
| TMEM67 | HAND2-AS1 | MAP1B | LOC100287387 | CLEC2L | LMOD1 |
| LINC01460 | LINC01936 | PLA2G4A | LINC01119 | MSC | CBR3-AS1 |
| MIR576 | PYGB | HSPA8 | FILIP1 | CNNM3-DT | DCAF8L1 |
| BABAM2-AS1 | GJC1 | ASB2 | FOXF2 | RARRES1 | LINC01087 |
| MYL10 | HTRA3 | ISLR | MTTP | SDC3 | MAP3K20-AS1 |
| COL4A2-AS2 | LOC100130691 | AARD | MYOSLID-AS1 | TMPPE | SNORA115 |
| LINC00346 | ANAPC7 | IGHMBP2 | CSPG4 | MGC16025 | MIR1204 |
| KITLG | LOC339593 | BTBD3 | MACROD2-AS1 | PIP5K1C | LINC01431 |
| SLC51B | ABL1 | NEURL1 | SNHG17 | GPR37 | KCNK15-AS1 |
| DBNDD2 | LOC105372631 | STAB2 | SLC8A2 | MIR1-1HG-AS1 | PXDN |
| LINC01697 | LINC01695 | SMYD4 | PLCD4 | SERPINH1 | C21orf62-AS1 |
| DENND2B | SYNM | COL18A1-AS2 | WFDC3 | GSTTP2 | LRRC61 |
| UBE2G1 | LARGE-AS1 | PTCHD1 | LINC00229 | LOC642648 | INKA1 |
| PDLIM4 | LINC00922 | AMOTL1 | SEMA3A | RAB41 | TMEM100 |
| KCNIP1-OT1 | OSBPL10-AS1 | KBTBD13 | KCNK4-TEX40 | ADGRA2 | LINC01614 |
| FREM3 | LOC100507144 | MIR8064 | CST5 | COL19A1 | LOC339902 |
| PRICKLE2-AS1 | PRICKLE2-AS2 | PRICKLE2-AS3 | LINC00877 | PDZRN3 | PARM1 |
| LINC00879 | LINC02044 | PARVA | BTC | CNN1 | CCDC63 |
| NXPH3 | SPATA4 | AADACL2-AS1 | TMEM14EP | LINC02006 | LOC101929106 |
| TPRG1-AS1 | SUCNR1 | LOC100130872 | PGM5 | SRC | LOC101929595 |
| IQCM | LINC01197 | CRYGC | GSG1L2 | MRPS6 | LINC01265 |
| OXCT1-AS1 | NNT-AS1 | MANBAL | LINC02731 | ACTG2 | LINC02113 |
| LOC285638 | LYPD6 | LOC105379152 | FOXP4-AS1 | WWTR1-AS1 | C5orf66-AS2 |
| NPY6R | LOC102724404 | LINC01845 | LOC728095 | MKX | LOC100505502 |
| GAS6-DT | ARRDC4 | LOC101928491 | GYG1 | CACNA1H | KCNB1 |
| CCND2 | CASQ2 | HAND1 | LOC101929705 | LOC100506188 | LOC101928540 |
| RNF150 | HHEX | LIN37 | LOC285762 | ATP2B4 | LINC01013 |
| LOC644135 | GPR34 | ITGA5 | ADRA1D | GPRC5A | MOB2 |
| IFIH1 | HOXA2 | PRDM6 | ADAM19 | SLC7A5 | HRAT92 |
| HOXA6 | PEX14 | RBFOX3 | XCL1 | SLC25A6 | SMPD2 |
| TNNC2 | PCA3 | OPN4 | COX6C | HTR1B | FOSL2 |
| RBMXL3 | MT1X | SCARA3 | MCHR1 | COL5A2 | LPP |
| MIR106B | EPHA7 | ROR1-AS1 | LINC00163 | ALDH1B1 | LOC100507516 |
| TPST1 | MIR2052HG | LOC102724804 | LOC101927066 | LINC00536 | HCFC1 |
| LOC101927822 | MYH11 | LINC01235 | C9orf92 | DUPD1 | MAMDC2-AS1 |
| GNA14-AS1 | LINC00476 | SAMD12-AS1 | LOC101928748 | PTGS2 | LINC00106 |
| FAM174B | PTCHD1-AS | RPS26P11 | DOK6 | ZFHX4 | GAS6-AS1 |
| GPC4 | MAP7D1 | ME1 | C14orf28 | ADAMTS8 | KBTBD8 |
| ZBTB44-DT | CUTC | CPXM2 | KCTD21 | ENGASE | QRFP |
| NT5DC3 | NHSL2 | LDLRAD2 | MMP3 | ARSI | LURAP1L |
| ECE1-AS1 | CCDC80 | MACC1 | MIR12128 | RASL11A | SPATA31E1 |
| TRMT61A | PCDHGC4 | CRISPLD2 | TSTD2 | PARM1-AS1 | NPAS2-AS1 |
| LINC00452 | LOC105376353 | IDO2 | COL6A1 | CD27 | EPPK1 |
| ASB5 | | | | | |

BoneMarrow

| | | | | | |
|---|---|---|---|---|---|
| DPEP2NB | LINC00115 | ANKRD30A | ACYP1 | CDC20B | LOC105378586 |
| UBE2E3 | EPDR1 | GPR101 | FCGR3A | CYSLTR2 | LYPLAL1 |
| RNASE1 | C11orf21 | PIK3CD-AS2 | PRR7 | CDCA3 | MANEA |
| SYCP1 | NPPA-AS1 | CCDC112 | OR2L13 | SDR16C5 | SHOX |
| CADM3 | TNFSF14 | CD53 | MYO1A | IL18R1 | ZFP36L1 |
| FCAMR | GLMN | ARHGAP9 | LOC101928163 | DNAH14 | TTC26 |
| UQCRH | LOC101928728 | SPACA1 | FRA10AC1 | FAM47A | TLR8 |
| MAIP1 | KLF18 | MS4A1 | PTGDR | CCR3 | GNG13 |
| TLR7 | SCAND1 | SP100 | LINC01685 | AGAP2 | IGLL5 |
| SMAP2 | TPTE | FAM72B | DYRK4 | TMEM154 | CHAT |
| TRAPPC4 | MCHR2 | C18orf54 | IQCB1 | MYB | ADGRE2 |
| H2AX | SAMD9 | STIL | ZDHHC22 | ODAPH | ZNF530 |
| BRCC3 | SMIM32 | RGSL1 | SS18L2 | BIN2 | CCDC169 |
| CD163 | TRMT61B | SLITRK4 | LINC00324 | TROAP | BCAP29 |
| VSIG4 | TUSC2 | LINC01360 | LINC02238 | SIGLEC10 | ERICH3-AS1 |
| DLSTP1 | LRRCC1 | IL15 | RFLNB | NUP54 | FBXO15 |
| IRF2BP2 | PCDHA5 | MAGOHB | COL24A1 | SFTA3 | PKN2-AS1 |
| CERKL | C1QL2 | CEACAM8 | CEACAM3 | CDK1 | CD69 |
| ARPP21 | CEACAM21 | EXO1 | PEX5 | CMC1 | LOC100129620 |
| FCGR2A | HSH2D | OLFM3 | MIS18A | NBPF6 | ZNF84 |
| GJA8 | ERAL1 | DRAM2 | LMX1A | NOL7 | CHIAP2 |
| LOC101928718 | LINC01750 | ANKRD30B | AP4B1-AS1 | ASPRV1 | MDH1B |
| CCT6B | VAMP1 | EMBP1 | LINC00869 | LOC100996724 | RPE65 |
| OLIG2 | CUTA | LILRB5 | LOC728989 | CMPK2 | MARCO |
| LOC100996740 | EXO5 | LRRC63 | PIK3CG | ZFP1 | ABI3 |
| STK16 | FLG-AS1 | PATL2 | MCEMP1 | FAAH2 | LOC101928009 |
| TMED9 | SAGE1 | TAFA2 | HLA-DRB1 | LILRA6 | COQ2 |
| BARHL2 | GJB2 | PARVG | ARHGAP11A | STXBP5L | CHORDC1 |
| ZFP30 | CD1A | SPAST | SYT4 | PLEK | IKZF4 |
| LILRA2 | TMEM217 | AADACL3 | LOC729867 | FCRL3 | SLC17A3 |
| CD48 | PPP1R35 | FCRL2 | NPL | C3orf67 | ZNF208 |
| ZBTB26 | SIGLECE | RPTN | LOC100505918 | MSR1 | ARAP2 |
| C1orf105 | LOC100506023 | HK3 | CENPL | ENTPD1 | GAPT |
| NDUFAF3 | OR2H1 | MTBP | SIRPB1 | SLC4A10 | ARHGEF6 |
| ECT2 | ERLIN1 | LOC647070 | SMG7-AS1 | PAX4 | MS4A7 |
| ICAM3 | OARD1 | CEACAM18 | ILDR1 | NKAIN3 | ZNF559 |
| TRIM10 | FEM1C | CLEC4C | BDNF | FAM201A | FOLR3 |
| WASF1 | RAB27A | ZNF569 | CCR2 | ZNF10 | LINC00467 |
| GFI1 | SP140 | TAGAP | CDKN1B | LILRB1 | ZNF580 |
| CEACAM4 | ZNF678 | IL10RA | LINC00582 | ZNF727 | CLEC12A |
| ZNF112 | LGALS8-AS1 | IRAK3 | SAMD9L | CENPM | GAS1 |
| IL7 | SPI1 | GLYAT | NOG | ACKR1 | GCSAML |
| ARL6 | ZNF738 | PIP4K2C | LINC02656 | CENPE | ZNF180 |
| PRKCQ-AS1 | PLAG1 | KIF23 | MGAM | CENPK | ZNF81 |
| LRTM2 | KCNA3 | SERPINB8 | SLAMF7 | DDIAS | ACD |
| FAM238A | FAM238B | ADIPOQ | ESM1 | PARTICL | TUNAR |
| METTL23 | ZC4H2 | LPO | CD58 | ARMC4P1 | ADAM18 |
| GPR15 | CCNA1 | EPGN | ZNF534 | LOC101929431 | POC5 |
| TBXT | FAM47C | MIR663A | LINGO2 | LOC441666 | SPSB2 |
| ZNF487 | ADO | PGLYRP1 | ZNF485 | HDC | AIM2 |
| RASAL3 | DUSP22 | CENPW | CYP4F3 | CLEC12B | COX11 |
| ANTXR2 | DDX21 | ZDHHC13 | MIR31HG | MUC22 | CLEC2D |
| USP17L13 | CCL3 | TMEM71 | LINC02671 | LMNB1 | ZFP37 |
| LILRA1 | ADGRL4 | GZMB | SAMD7 | DNAJC9-AS1 | FIGNL1 |
| NETO1 | SLC36A4 | MKI67 | MNX1-AS1 | FAM111B | MCL1 |
| TRPM2 | MBL1P | ZWINT | BCL2L15 | SH2D4B | GJB6 |
| PYHIN1 | PIGP | CXCR4 | RAD51AP1 | TRMT12 | COCH |
| ANO5 | RAB24 | LAIR1 | IL37 | RAB27B | ENTPD1-AS1 |
| CC2D2B | ZNF518A | GLIPR1L2 | CAAP1 | PPP1R42 | LINC01475 |
| TYROBP | LILRA5 | ZNF266 | SGO2 | OSTF1 | CXCL5 |
| TRIM40 | CYB5R4 | CASR | NLRP3 | LHX8 | ZKSCAN7 |
| C19orf84 | CASP8AP2 | ASAH2B | TREML2 | RMI1 | PCDHA9 |
| RPA2 | MIR3663HG | FKBP1B | RNF166 | RUBCNL | SAMSN1 |
| PSMC3IP | SIGLEC5 | COL8A1 | RGCC | CLRN1-AS1 | LOC399815 |
| SHLD1 | KISS1R | HLA-DQA2 | CAPS2 | MUC16 | CRISP3 |
| NDUFB6 | PKIB | ANKRD18B | WDFY3-AS2 | NCKAP1L | SOX17 |
| ORMDL3 | ARG1 | C12orf42 | ZNF253 | ADGRE3 | FCRLA |
| LINC00466 | AXDND1 | SUPT4H1 | OLFM5P | ZNF501 | VSTM2A |
| IL17RA | CRYBA2 | GVINP1 | INPP5D | FSD1L | FBXO5 |
| HMGA2 | FGF5 | NXPE1 | OTX2 | EPB41L4A-DT | HRH2 |
| TLR10 | MRPL11 | FOXD3 | CCDC168 | PRELID1 | ELANE |
| HAL | FANCB | SNHG1 | PGLYRP3 | NBPF20 | TMEM102 |
| ITGB2-AS1 | POTEC | GABRG1 | ZNF829 | SPATA48 | ZNF729 |
| LOC440040 | LOC441601 | MIAT | DNAAF2 | UNC5A | NLRC5 |
| MS4A14 | IKZF3 | LINC00301 | LIPF | NIFK-AS1 | MUC7 |
| KIR3DL2 | KDM4D | CLEC4E | POM121L2 | GZMK | NROB1 |
| CD84 | CAMP | DPAGT1 | MIF4GD | GCNT1 | ARMC12 |
| CBWD3 | LOC100133315 | ZNF684 | CRISP2 | NKG7 | MAGEA11 |
| CYLC2 | C11orf80 | MGAT2 | C2orf78 | PRDM13 | ZNF229 |
| ANAPC15 | TATDN3 | NGB | ACBD6 | UBE2D1 | ZNF19 |
| HLA-DOB | MMP27 | LOC283214 | STARD6 | MZF1 | RSPO2 |
| KRCC1 | BCL11A | NCR2 | EPHA5 | TRIM53AP | TRIM51EP |
| SERPINI1 | IKZF1 | LOC100129203 | LINC01551 | SCEL | IL18RAP |
| DOK3 | SIGLEC7 | C11orf65 | PTPRC | NMBR | C16orf54 |
| CSTF2 | SSNA1 | CDH18 | SYPL1 | HTR3A | LOC101928940 |
| PCDHB16 | ALAS2 | PGLYRP4 | RPS6KA6 | PCDHA2 | GADD45A |
| ZNF200 | MUC4 | MRGPRX2 | HLA-DQB2 | ZIK1 | WDR87 |
| SNCB | MRVI1-AS1 | LOC101929538 | MED21 | TMPRSS11D | MNX1 |
| KNSTRN | S100A9 | SLPI | SOX1 | CREG1 | MMP9 |
| HMGN4 | CD1E | ZNF221 | USP17L3 | ZFP90 | MRFAP1 |
| LOC101927378 | MSRB1 | CEACAM5 | MIR200CHG | NECAP1 | LOC374443 |
| LINC02470 | CLEC12A-AS1 | ELOVL3 | RP1 | HINT3 | KLRA1P |
| MTIF3 | ZPBP2 | CLCA2 | SLCO1C1 | CD40LG | VWDE |
| FYB1 | LINC01572 | HMSD | SYCE2 | ADAM28 | FUJ13224 |
| SFTPD | FOXJ2 | CCL18 | LINC00938 | SOX4 | MARCHF8 |
| LRRD1 | AKAIN1 | IRF4 | MFSD1 | MAD2L1 | EOMES |
| BCDIN3D-AS1 | HPSE | ANKRD30BL | ZNF439 | KRT73-AS1 | CRYGS |
| TESPA1 | HLA-DMB | C16orf95 | TFCP2 | PPT1 | HPF1 |
| CCND3 | NBPF19 | HCK | OTP | ERG | ZNF583 |
| KYNU | FRAT2 | SSBP1 | GYPA | ABHD5 | HTR1F |
| GSX1 | LINC01465 | POLE3 | FPR3 | IGFL4 | FOXD1 |
| DACH2 | CLEC17A | TRHDE-AS1 | IL1RL1 | SGCZ | LINC02458 |
| TGFB1 | SELL | ATP2B1-AS1 | MMP8 | LOC101928617 | GTF2A2 |
| ZNF616 | TRPC3 | LNCOC1 | PPOX | ZNF596 | FAM133B |
| SLAMF6 | CBLN4 | VAMP4 | IDI1 | GRK6 | NPY2R |
| SLC27A2 | DALRD3 | ZNF224 | ZNF75D | GPN3 | FAM238C |
| IL10 | CEACAM7 | P2RY12 | LHX3 | NEDD1 | ZMAT2 |
| CBLN1 | EXOSC9 | PSMB9 | LOX | LINC02347 | LOC100128276 |
| ZNF257 | TPTE2 | ANKRD20A9P | ANKRD26P3 | RFC3 | MIPEPP3 |
| PLUT | PRKAG1 | ZNF268 | CA1 | EXOSC1 | MCM8 |
| INSYN2B | BARHL1 | LINC00437 | FOXA1 | ZNF560 | TLX1 |
| CKAP2 | LINC02338 | TOMM6 | OR7E156P | TLR2 | RBM11 |
| ADCYAP1 | RBM26-AS1 | LINC00410 | LOC101927284 | GLI1 | DOCK9-DT |
| GFY | ZNF43 | THAP2 | TTK | LINC00368 | LINC00354 |
| LINC01043 | CEPT1 | ICAM5 | GABRA2 | KLRC1 | DBF4 |
| PIP4P1 | ZNF492 | TENT5C | ADAMTS20 | VGLL2 | ADAD1 |
| NANOG | NKX2-1-AS1 | CHEK1 | NXF3 | LINC00645 | FRG2 |
| LOC101927124 | ZNF449 | LINC00648 | LOC105370489 | LILRB4 | ZNF720 |
| NAA20 | NAT16 | OTX2-AS1 | TMEM215 | AMTN | ATF4 |
| PCDHA10 | ZNF682 | KIF22 | TMEM196 | CXCL10 | LOC646548 |
| LOC101928075 | TFEC | ACOT6 | CCDC103 | ZNF724 | CCNB1 |
| NME8 | NEUROD1 | RTL3 | ABCA13 | EGR4 | USP17L8 |
| DKK2 | SNHG10 | LOC107984703 | LINC02325 | LINC02312 | HACD4 |
| SELENOH | JUN | LILRB2 | IL1B | IL1A | HOXB7 |
| ZNF480 | ADAM6 | LINC00221 | CHEK2P2 | NF1P2 | LINC01193 |
| LINC02203 | FAM30C | LOC646214 | CXADRP2 | PWRN2 | IPW |
| ZNF382 | CLEC5A | ZNF215 | NCR3 | FBXL15 | LINC02249 |
| OSGEPL1 | HERC2P10 | NMU | LINC02256 | PPIAL4H | CDC25C |
| CYP4F2 | KDM7A | GINS3 | IRAIN | TRAF3IP3 | ST8SIA6 |
| TSN | FCGR1B | LILRA4 | NFE2 | MPEG1 | OCSTAMP |
| PAX1 | PFKFB2 | LYSMD2 | TARP | CBLN2 | MARCHF4 |
| WDR77 | LYZ | LOC107984784 | KCTD12 | CGAS | HLA-DRA |
| LOC101928988 | SLC66A1L | DZIP3 | JAK3 | SYT16 | LY6H |
| URAD | HMX3 | ZWILCH | HMMR | DHRS9 | FAM216B |
| LTA | ZNF595 | TLE7 | CKAP2L | C2orf76 | SLC5A7 |
| SHCBP1 | C6orf52 | BAD | ZCCHC3 | MGAT4FP | KLRG1 |
| ZNF85 | TMED3 | UBE2Q2P2 | FOLR2 | ADAM2 | ZNF420 |
| SPX | DYNLRB2 | FOXN2 | TUBA4A | MIR9-3HG | SLCO4C1 |
| TCL1A | IRF2BPL | PRC1-AS1 | CRAT37 | LINC02207 | NPIPB8 |
| PRKXP1 | SRP14 | ZNF165 | KIFC1 | WNT8B | HCFC1R1 |
| MMP25-AS1 | ZNF286A | MS4A6A | FOXQ1 | ARMH1 | ASCL2 |
| MYEF2 | HOMER1 | KLHDC1 | SYT10 | GAD2 | ZNF585A |
| CCNYL3 | NFE4 | LOC101927814 | EFNA3 | IL21R-AS1 | ANKRD31 |
| RHAG | RUNX3 | TEX38 | ZNF688 | MBL2 | MIR762HG |
| PRDM1 | SULT1C3 | TRIM49 | LINC02190 | YBX3P1 | LOC105369266 |
| ENPP7P13 | ANKRD26P1 | MUC19 | AREG | CES1P2 | QRSL1 |
| CD244 | GABRG2 | ZNF624 | CNR1 | FAM122A | LINC02141 |
| GRK1 | RHOH | TREM1 | ZMYND10 | PROKR1 | ZNF582 |
| RGS18 | LOC104968399 | HCST | LRAT | ARHGAP19 | C5orf51 |
| SIGLEC12 | CD2 | CD1D | TTC14 | EEF1AKMT3 | LRRIQ3 |
| KIFBP | LIPJ | LIM2 | NOA1 | FBXO4 | SMC2 |
| PRMT5 | CCIN | CD79A | WDR53 | CDCA8 | LOC100287072 |
| FUJ36000 | ALG3 | S1PR1 | SLC25A21 | CD3G | CMTM8 |
| SCN10A | AOAH | ID1 | GYPE | RAB13 | ASPHD2 |
| STK26 | PTPN22 | NIPSNAP3A | PLAC8 | CEBPE | THCAT158 |
| C2orf15 | NKX2-1 | LOC101927166 | LOC102724596 | VAV1 | FAM76B |
| RB1-DT | NXPE4 | TNFAIP6 | MSX2P1 | USP17L15 | ADAT1 |
| LAPTM5 | CRH | EFCAB3 | ZNHIT2 | GCA | ZFP69 |
| CCNYL4 | ARHGAP15 | CITED2 | KCNJ2-AS1 | HTR2C | FCGR3B |
| CXCL9 | DUS2 | SLC6A14 | RUNX1 | HEXIM1 | BTLA |
| ZFAS1 | YEATS4 | C5orf47 | MEIKIN | CAGE1 | CD177 |
| KLHL1 | DEFA3 | CBX3P2 | SLCO6A1 | LINC00667 | TOPAZ1 |
| LINC-ROR | KCNC2 | LOC644669 | SLITRK1 | C9orf43 | TTC39C-AS1 |
| PRPS2 | ATOH1 | CYP26A1 | ZNF830 | GCSAM | GPR174 |
| EDA2R | ITGAM | CD5L | ZNF780A | LINC01924 | LINC01538 |
| CXCR2 | LOC643542 | TARM1 | LINC01541 | MRPL40 | EPOR |
| PLA2G7 | LOC284241 | ANP32E | ZNF502 | PTTG1 | CLEC11A |
| ZNF391 | DHFR | ZNF300 | SERTM1 | HMBS | ADGRE4P |
| PCDHA6 | CD33 | NPEPPSP1 | STK17B | WAS | ZNF700 |
| KRTAP10-11 | KRTAP10-7 | MISP3 | NHEJ1 | CIB3 | GPR65 |
| LY9 | GPR88 | ZNF273 | ZNF626 | JUNB | LOC400684 |
| C8orf34 | NUDT12 | LINC01838 | NCR1 | MYO3A | SIRPB2 |
| MPO | PCLAF | ZNF571 | FCER2 | SLC6A16 | CLEC1A |
| FCRL1 | LYPD4 | C3AR1 | HTRA2 | LOC100505715 | RAG1 |
| ZNF233 | OR51B5 | ANKRD18A | PROKR2 | S100A8 | CARD8-AS1 |
| KHDRBS2 | PSMB10 | PTPN6 | TBC1D8B | LOC400710 | RGS19 |
| ITGA4 | CYBA | SIGLEC17P | SIGLECL1 | LINC01872 | CXCR1 |
| RELL2 | JTB | KIR3DX1 | LILRP2 | CD163L1 | HEPACAM2 |
| CHIT1 | NLRP13 | ARHGAP36 | CDRT15L2 | LINGO3 | ARHGAP11B |
| LINC01249 | LINC01247 | LOC100506274 | SKOR2 | FAM30A | RSRC1 |
| SLC18A1 | LINC00276 | FLG2 | BET1 | ADGRE1 | MS4A3 |
| KCNK12 | ASCL1 | DRGX | ZNF223 | RRS1 | PIK3R3 |
| HS3ST4 | KLRD1 | HES7 | LINC01833 | ID2 | LOC100129434 |
| MIR4432HG | LINC01800 | LOC100996318 | SNX3 | TMEM203 | M1AP |
| FUNDC2P2 | CCDC15 | ST3GAL5-AS1 | OR7D2 | ANKRD36BP2 | LOC101927050 |
| FAM95A | ANKRD20A8P | LINC00342 | CALCR | NCAPG | CADM2 |
| USP17L9P | RGPD4-AS1 | DEPDC1 | CX3CR1 | SH3RF3-AS1 | PCDHB6 |
| SPRY3 | NDUFS2 | ZNF781 | PGM5P4 | RPL23AP7 | CNGA4 |
| LOC107985820 | COA6 | PRG3 | NDC80 | HBA1 | LINC01854 |
| FAR2P1 | MADCAM1 | FAM201B | LOC101928386 | PABPC1P2 | DUT |
| ADAM20 | ADAM21 | CREBZF | CD1C | BCL2A1 | PRSS38 |
| ZNF182 | FAM133DP | CCDC148 | LOC101929532 | SCN1A-AS1 | MPZL3 |
| CDCA2 | HELT | SIRPG | RSPO3 | NLRP4 | GSDMC |
| TMEM243 | TAF1L | LINC01934 | FAS-AS1 | ZNF222 | MS4A13 |
| SRGN | EFCAB9 | NEFM | TIMM29 | LINC01790 | LKAAEAR1 |
| ANKRD44-IT1 | CCNYL2 | LOC100288203 | PRAMENP | CTXN2 | MNDA |
| CLEC4D | PDE3B | SPAG16-DT | SERPINB10 | GABRB2 | LYSMD1 |
| GATA1 | ARF6 | CT75 | MED12L | H2BC21 | OR4N2 |
| MGAT4D | RNF141 | CYTH4 | ST8SIA4 | ECEL1P2 | ZNF23 |
| DCST1-AS1 | SLFN12L | HJURP | LINC01881 | CD300LB | SIRPG-AS1 |
| CLEC4G | PLK4 | IL7R | SLC25A51P4 | BTG1 | BRS3 |
| ZNF337-AS1 | LINC01733 | FAM182A | FRG1BP | BPIFA4P | RALY-AS1 |
| RPL23A | PCDH8 | SLFN14 | BHLHE23 | ZIC2 | FGF23 |
| SLC12A5-AS1 | ZNF334 | NHLH1 | FCMR | NBPF4 | LOC729296 |
| MIR646HG | TREML4 | TRH | SPDL1 | ANKRD30BP2 | ABCC13 |
| BMP10 | LINC01684 | TRHR | POU4F1 | LINC00945 | CSNKA2IP |
| RUNX1-IT1 | G6PC2 | DSCR10 | MS4A4A | BRWD1-AS2 | ERMN |
| TRPM2-AS | ZNF567 | H2AZ1 | LINC01311 | NME3 | WNT1 |
| ORC6 | FCRL5 | PASD1 | FAM71C | APBB1IP | HMX2 |
| FAM133A | TEX28 | ODF3B | NUP50-DT | NEXMIF | OR1F1 |
| RPL23AP82 | LINC01266 | POU3F4 | MRPS34 | NEUROD2 | TECRL |
| TEC | LINC00690 | ZNF736 | GABRA6 | GABRA4 | C3orf35 |
| GFRA4 | MFSD3 | MOXD2P | DUSP28 | TBX21 | LARS2-AS1 |
| BAGE | AZIN1-AS1 | GPR141 | TNIP3 | CA3-AS1 | SOX18 |
| HES4 | ZMAT1 | FPR2 | ZNF716 | DLX6 | NKX2-3 |
| FOXC2 | FLG | CTSG | FCN1 | CSN1S1 | CR2 |
| CNR2 | CKS1B | MAGEF1 | SUCLG2-AS1 | ESCO2 | LINC00971 |
| FAM172BP | LINC02085 | CLDN5 | LINC01205 | LINC02042 | H1-10 |
| CEACAM6 | C1orf56 | NUP210P1 | DNAJB14 | PCLO | CD37 |
| GKAP1 | C10orf53 | LOC339874 | NPHP3-AS1 | LINC01210 | TMEM158 |
| STXBP5-AS1 | JCHAIN | GNS | LOC646903 | TEX13D | SERPINB2 |
| TIPARP-AS1 | LRRC77P | EGFEM1P | LOC100128164 | ZNRD2-AS1 | KRT9 |
| ELOVL4 | LINC02015 | KLHL6-AS1 | KRTAP5-9 | SLCO1A2 | ADIPOQ-AS1 |
| RRAGA | FCRL4 | LINC01793 | DARS-AS1 | MLNR | CLECL1 |
| ANKRD18DP | ZNF718 | NDST3 | LINC01097 | CPEB2-DT | GP9 |
| G2E3 | LOC105370708 | LOC105370687 | LINC02261 | LOC439933 | SLC4A1AP |
| TPD52L3 | LOC401127 | PTX4 | LINC02265 | POU4F2 | LINC02232 |
| FTLP10 | IL17C | TNF | RAB32 | ZNF572 | CST7 |
| LOC101928978 | S100P | CXXC4-AS1 | RPL34-AS1 | GPR139 | IFITM3 |
| PCDHA8 | C14orf39 | OLIG1 | STPG2 | UGT3A2 | PSTPIP2 |
| C11orf97 | METTL14-DT | ALG10 | KLRF2 | LINC02465 | LOC101927359 |
| LOC107984036 | CRNDE | OTUD1 | MRGPRX4 | NEFL | MIR4453HG |
| PCDHB9 | NLRP14 | ATP23 | C1orf74 | TTC9B | C9orf40 |
| LINC02427 | RAG2 | MIR3945HG | LINC01093 | LINC01667 | SSTR3 |
| MRPL41 | CTD-2194D22.4 | LOC401176 | LINC02218 | LINC02241 | LINC02109 |
| LINC00909 | CAPSL | LINC02119 | LINC02108 | PCDHA1 | ZNF491 |
| ZNF441 | EID1 | CTD-2201118.1 | LINC00461 | HOXA10 | BARX1 |
| ARRDC3-AS1 | MCTP1 | PRICKLE4 | RGMB-AS1 | LINC00491 | LOC101927190 |
| MTNR1B | ANKRD34C | CBWD5 | SKA1 | UBQLNL | ZNF471 |
| PCDHB17P | PCDHB18P | PCDHB19P | TCN1 | IMP3 | SIGLEC8 |
| ZNF300P1 | SERP1 | LOC100128059 | ANKRD34B | POU3F2 | UGT3A1 |
| GOLT1B | CTC-338M12.4 | LINC00639 | LOC101927950 | LOC730183 | C1orf158 |
| C8orf37 | MAFA | LOC285819 | ZSCAN12P1 | ZSCAN16-AS1 | ZNF230 |
| LINC01015 | HLA-F-AS1 | ZNRD1ASP | NEUROG3 | SIKE1 | MICB-DT |
| TBX18 | LOC100294145 | HCG24 | HCRT | NPBWR1 | XCR1 |
| GCLM | ST8SIA3 | CEACAM22P | CXCL1 | ADCY10P1 | TREML3P |
| USP17L19 | USP17L17 | TALAM1 | AMPH | ZNF519 | PDX1 |
| GABARAP | CCR1 | MANEA-DT | LOC101927314 | ZNF66 | GPR6 |
| ADGB | RNF217-AS1 | LOC153910 | LOC101928661 | RAC2 | SLC25A38 |
| TEX13C | LOC101929297 | LOC100289495 | TEX12 | AMPD1 | CCDC175 |
| DBX1 | PRSS16 | INSM1 | LHX1 | PKHD1L1 | CHMP5 |
| LY86-AS1 | THAP10 | GRK2 | DDIT4 | POU4F3 | ZSCAN5C |
| LHX5 | DUSP12 | GABRA1 | USP17L2 | SMIM30 | POM121L12 |
| GZMH | KCNV1 | ZIC3 | KCNQ5 | NCR3LG1 | DNAJB9 |
| XCL1 | PAX9 | FAM98B | JAZF1-AS1 | ZNRF2P1 | NPSR1-AS1 |
| AOAH-IT1 | FAM183BP | LUARIS | BMP3 | UTF1 | VWC2 |
| LOC401357 | LOC102724738 | LOC100128885 | LOC101929322 | GS1-124K5.4 | WDR36 |
| PRELID3B | AVPR1A | C5orf34 | EPB41L4A-AS1 | LINC01555 | LOC100128317 |
| DPY19L2P4 | LOC101927446 | TLX3 | SLC25A40 | NCF4 | NPIPB6 |
| KMT2E-AS1 | NXPH1 | SCRT1 | SLC2A13 | LINC02476 | POT1-AS1 |
| CA10 | NOP10 | CHSY3 | HTR1A | HOXD13 | HOXA13 |
| TMEM253 | FOXE1 | CLEC9A | OR2A1-AS1 | NPY5R | C17orf77 |
| B3GAT2 | BTK | RIPK2 | LINC01003 | MYOD1 | MEFV |
| BLACE | CYTIP | GALR2 | GPR84 | FSCN3 | ZNF831 |
| LOC101929294 | UOX | LINC02209 | TCHH | SLC22A16 | ASNSP1 |
| LINC00293 | PCDHA4 | RPAP2 | CCR5AS | EREG | LOC100130298 |
| LOC102724612 | LINC01289 | LOC392232 | FCRL6 | DEFA4 | ZNF677 |
| SLC17A6 | KCTD8 | LOC105375690 | LTC4S | RAD21-AS1 | LOC101927543 |
| PCDHB7 | LINC00861 | PTCSC1 | ZNF354C | SLC18A3 | HASPIN |
| UBQLN2 | ZNF484 | LAIR2 | ZNF658 | DEF6 | CCDC182 |
| ZNF252P | PELO | LRIF1 | PTPRD-AS2 | ATP8B4 | SIX6 |
| KIF20A | PRAMEF2 | ZNF658B | GXYLT1P3 | LOC101928381 | LOC107984035 |
| FGF7P6 | LOC100132249 | PTGER4P2- | FRG1JP | LOC101927069 | RHOB |
| | | CDK2AP2P2 | | | |
| SMC5-AS1 | NEUROD4 | TRIM58 | C4orf17 | CARNMT1-AS1 | RPSAP9 |
| LOC101927450 | LOC105376114 | LOC642943 | PTMAP11 | LOC286359 | LINC01410 |
| SMC2-AS1 | CIP2A | TXNDC8 | B3GNT10 | COX10 | LOC100505588 |
| LRRC38 | CLVS2 | CCNA2 | C1GALT1C1 | BBS12 | OTUD6B-AS1 |
| CHGB | FAM230C | LOC100505874 | SOX11 | PEX3 | PCDHB14 |
| PCDHB13 | NEUROG2 | HOXB8 | RAD21L1 | LINC01560 | MIR137HG |
| CARNMT1 | TACR3 | UTP18 | XK | MANF | CHMP1B2P |
| NXF4 | NKAPP1 | ANXA1 | HTR5A | DNTTIP2 | TRPA1 |
| MARCHF11 | IL12B | ZNF613 | SLC32A1 | LOC388813 | FRG1 |
| ZNF286B | PRSS45P | LOC112268124 | CCDC173 | FAM81B | EMB |
| SNHG31 | DOCK8-AS1 | S100A7A | SCPEP1 | PCDHB11 | PCDHB10 |
| SOGA3 | SOWAHD | MGAM2 | BLOC1S4 | PCP4L1 | CDC14C |
| LRRIQ1 | NRARP | PRAMEF12 | ADRB2 | LIPK | SHISA3 |
| UNCX | ACSM4 | ZNF99 | OR2T5 | OR13A1 | PPM1N |
| DBX2 | CCDC158 | PLPPR5 | SYPL2 | GJB7 | IL22RA2 |
| MIS18BP1 | TCHHL1 | LINC00612 | LRRC26 | LOC100507412 | ZNF599 |
| THEGL | SPTA1 | CA13 | NKX1-1 | SOX14 | ZNF470 |
| LOC339862 | LOC401478 | TFAP2D | OR7C1 | C11orf87 | CCDC140 |
| COL25A1-DT | SEC31B | IER5L | PRR27 | OR10P1 | GMCL1 |
| ZNF709 | TMPRSS11B | BHLHE22 | BIRC8 | ENTHD1 | ITLN1 |
| ELMOD2 | FEZF2 | LAMTOR1 | TMPRSS15 | A4GNT | CYP4Z1 |
| P2RY8 | RNASEL | H3P4 | H2BP2 | CDK5R2 | ZNF93 |
| LOC101929141 | TMEM271 | RNF175 | CYBB | LINC02875 | ITK |
| GLA | | | | | |

Brain

| | | | | | | |
|---|---|---|---|---|---|---|
| MCF2L | OXCT2P1 | DSCAM | UQCRHL | KIF3A | ASTN1 | CNTNAP4 |
| OPRL1 | KLHDC4 | NFAM1 | ADGRL3 | TBC1D10C | ZIC4 | RPH3A |
| KIF1A | SEZ6L | EPHB2 | SHTN1 | USH1C | FAM161A | MARCHF1 |
| CABP5 | ZNF365 | WDR76 | KIRREL2 | LINGO1 | KCNJ2 | RBP3 |
| DNMT3B | ZNF415 | ACVR1C | SFTPC | RSU1P2 | LMF1 | FUT9 |
| ATF3 | KIAA1324L | ABCA12 | ADCK1 | SMYD1 | TMEM63C | NAV2-AS5 |
| KLK6 | FSTL5 | EFNA2 | CARD11 | FRMPD2B | TRIM49C | AP3B2 |
| MED27 | ADGRE2 | CRB1 | TEX14 | CLEC12B | SORCS3-AS1 | NCKAP5 |
| GSG1L | SNORA110 | WFIKKN2 | RNA5S9 | SH3GL3 | MMP16 | PAUPAR |
| LINC01647 | TMEM53 | GRIK2 | SLC6A1 | CNDP1 | CSRP3 | HNRNPKP3 |
| OPALIN | ACTRT2 | SLC5A11 | SPECC1 | BMS1P2 | DOCK10 | PCDHAC2 |
| RGR | ZDHHC22 | YPEL4 | GRM8 | PLPP4 | APBA1 | ADAM23 |
| KAZN-AS1 | IDNK | IER3 | COBL | FRMPD2 | PCDHA9 | MEGF10 |
| AGAP1 | LMO1 | INSYN2A | NRXN1 | ARHGAP22- | MYO18B | CADPS |
| | | | | IT1 | | |
| C10orf90 | NOS1 | C12orf56 | CFAP53 | ARHGAP22 | MIR3663HG | MYO16 |
| LOC440570 | XRCC4 | GABRB3 | TEKT3 | RTN1 | DCT | DNAH10 |
| ST18 | PEX5L | PLD5 | IL25 | STX4 | GAL3ST1 | ZDHHC11B |
| LINC01654 | LOC101926964 | DPF1 | LINC00200 | ILDR2 | PTPRD | ADGRA1 |
| TIAM1 | BRINP1 | LOC100422212 | ADARB2-AS1 | TIMM23B | DMBT1P1 | BRINP2 |
| PPP2R2B | OPCML | ARAP2 | LINC00700 | ANKUB1 | KIAA0556 | TMC7 |
| PPFIA2 | PIN1P1 | APBA2 | LINC00701 | NPAS3 | NOVA1 | NMUR2 |
| PPP2R2C | DPP6 | LINC01657 | TMEM132D | PTPN11 | LINC01163 | LYZL4 |
| NFASC | CENPJ | DENND1A | FAM205C | CLIP2 | LINC01164 | EFCAB8 |
| TMTC4 | FBXO15 | LINC01645 | PRKCQ-AS1 | DNTT | PLA2G4C | KIF19 |
| CELF4 | PCDHA3 | P2RX7 | LINC02648 | RPS6KA2 | TMEM171 | SLC4A8 |
| SLC39A11 | PCDHA5 | FAM131B | BMS1P20 | ADCY8 | PINX1 | C4orf47 |
| GAB4 | CD207 | TMEM145 | PCDH15 | WNT9A | LINC01166 | CACNG4 |
| DDR1 | SLC1A6 | CTNNA3 | LRTM2 | KCNK10 | ADGRA1-AS1 | SLC5A4 |
| C21orf62 | HAPLN3 | CD22 | ARMC3 | PPP3CB-AS1 | KCNG4 | DCAF4L2 |
| SPOCD1 | ARPP21 | SCN1A | NCAM2 | NETO1 | CFAP161 | GHRH |
| ZNF488 | MAL | ZNF806 | CDH19 | CLRN3 | DNAJC12 | CFAP97D1 |
| OTUD7A | NRXN2 | NRXN3 | TUBB4A | RPEL1 | GRIN2A | MC4R |
| AKAP6 | CACNG6 | ASB4 | ACSL6 | SLC8A3 | TGFA | ELAVL3 |
| NELL1 | EPHB1 | SYT3 | DLGAP2 | GRIA2 | ADD2 | SHANK2-AS1 |
| CLDN24 | PPP1R17 | PRAME | NENF | USP17L7 | IGSF11 | FLJ42102 |
| MYT1L | CADM1 | LMLN2 | GPR15 | GRID1-AS1 | DMBT1 | NGB |
| LUZP2 | OTOP3 | TMCC2 | LINC01517 | KLHL32 | VSTM2A | TOX2 |
| CSMD2-AS1 | KCNQ2 | NOVA2 | LINC00837 | MOBP | LRRN1 | KCND2 |
| CTNNA2 | CCNQ | SLC26A9 | GRIA4 | IGSF21 | PDE6D | ADGRB3 |
| ANLN | CRACD | EVI5L | GRM5 | RTF2 | KCNJ10 | HS3ST5 |
| EPHA10 | SRCIN1 | CACNA1E | SNORC | CLCA4 | CACNA1B | XKR6 |
| RGS7 | GPAT2 | LRP1B | CCDC144A | SLIT1-AS1 | DSCAML1 | ADARB2 |
| TRIM53AP | MYT1 | TMEM132B | COL22A1 | TMEM114 | LOC101559451 | NCAM1 |
| C2orf92 | MYBPC2 | LINC00943 | NAT16 | ZNF536 | LOC339166 | SALL3 |
| SLC10A2 | BPIFA1 | LINC02405 | LOC101927690 | TGFBRAP1 | ACAN | FAM135B |
| PPP2R3B | KCNJ9 | LINC02393 | SUMO1P3 | RTP2 | ZNF280A | TTYH2 |
| LINC01551 | SPP1 | TMEM132D- | LINC02322 | PTPRN2 | AGTPBP1 | DSTNP2 |
| | | AS2 | | | | |
| SCEL | PTPRZ1 | LINC02415 | PCDHA10 | CPEB1-AS1 | KCTD13 | PAK5 |
| GLTP | SPATA16 | LINC02414 | WASHC5 | LOC338963 | ANKRD24 | EIF2S3B |
| PCSK6 | PNLIP | GRK6P1 | LRP12 | LINC00052 | ELOA2 | LINC02446 |
| MGAT3 | KIF4B | LINC00463 | DACT3 | NTRK3-AS1 | IGLON5 | ENPP2 |
| CDHR1 | CECR2 | LINC01053 | GRID1 | LINC01580 | USP32P1 | CACNG3 |
| PHACTR3 | CNTN2 | CHRFAM7A | PREX1 | KLRC3 | KRT16P2 | CTNND2 |
| HMX1 | CDH26 | RALGDS | GNG2 | BRSK1 | KRT17P1 | FAM238C |
| TF | CAPN9 | LOC101929259 | PSAT1 | LOC100507472 | TBC1D27P | LIPE |
| NCAM1-AS1 | PRKCQ | PTN | USP17L4 | LMF1-AS1 | KRT17P2 | TAFA5 |
| WASF3 | RASAL1 | FAM124A | USP17L1 | BPIFB6 | CCDC144CP | CDH7 |
| PKMYT1 | MIR124-2HG | KRT6B | LINC00642 | GPA33 | NOS2P3 | FAM170A |
| ACTR3B | ABCA2 | CTAGE11P | UNC79 | ABCG1 | LOC100287072 | PDE6B |
| HTR3A | MMD2 | ATP10B | WNT7A | NPSR1 | CDH6 | CNTNAP5 |
| KREMEN2 | PLPPR1 | LOC101927248 | LINC02318 | FGA | CDH22 | CDH10 |
| GRIK5 | LINC02762 | KLHL14 | ACTC1 | LINC02177 | CDH12 | VSTM2B |
| KCNH1 | CSPG5 | NALCN-AS1 | PTPRS | ETV5 | DNAJC6 | PCYT1B |
| ABRAXAS2 | INPP5A | DPYSL5 | TMIGD2 | TMEM151A | CAMKMT | MDGA1 |
| PCDHA2 | TSGA10 | TEX29 | TMEM150B | ATP13A4 | TNFRSF13B | MDGA2 |
| SYNDIG1 | SORCS3 | LINC00354 | LINC00929 | LINC01567 | C1orf94 | ANO4 |
| RDH8 | HHATL | SOX1-OT | PDCD6IPP2 | RHAG | PRKN | SYT16 |
| PANTR1 | SCN3A | LOC100506016 | AK5 | CHRNA1 | CPPED1 | STMN4 |
| GOLGA6L7 | CACNG8 | LOC101928730 | SPHKAP | DNAI2 | PPP1R16B | INSYN1 |
| LOC101929473 | FUT8 | GRID2 | LOC646652 | CALN1 | TBC1D14 | JAKMIP3 |
| LOC101929653 | BRI3BP | ST6GALNAC3 | LOC105370757 | CLCC1 | RUNDC3A- | SLC22A6 |
| NTM | LRIT2 | JAKMIP2 | CFAP20 | LINC02133 | NALCN | PKP4 |
| LOC283177 | TULP4 | LXN | FAM222A | NPFFR1 | CLVS1 | A1BG |
| TUBB3 | TRPV4 | ADAMTS20 | ASB10 | XKR4 | LINC02075 | LINC02125 |
| KLK7 | TRIM64 | TFF3 | FAM189A1 | GABRG2 | SPPL2C | SPRED3 |
| TMEM161B | FAM222A-AS1 | SPOCK1 | RASGRF2 | CNR1 | LINC02071 | AMER3 |
| MXD1 | C22orf34 | AJAP1 | PLPP2 | GPIHBP1 | LINC02072 | CDCA7 |
| FAM171A1 | HARBI1 | DSCAM-AS1 | NLGN4X | MHENCR | WSCD1 | RELN |
| ELFN2 | GNAZ | ZIC1 | PHF21B | BPIFB3 | LINC01520 | IL12RB1 |
| LINC01561 | HPN-AS1 | FOXO6 | ZNF71 | CDH8 | HSF5 | DNAH11 |
| MEX3D | TMPRSS5 | UNC5C | LOC100289473 | PRIMA1 | SEMA3E | SEC14L5 |
| KCNH8 | PLEKHH1 | ACSBG1 | CSMD1 | LOC101928107 | CST1 | PBOV1 |
| LOC101928251 | CDH20 | CADM2 | PRNT | DSCR4 | HAPLN1 | IRGC |
| LINC00469 | LINC00320 | LINC01102 | LINC00658 | TIGD4 | CAPN13 | KCNA10 |
| JAKMIP1 | CHP2 | LINC01103 | LINC01713 | LINC01671 | CCDC8 | EDAR |
| HID1-AS1 | PSG8-AS1 | LINC01114 | LAMP5-AS1 | SMOX | SHISA7 | LOC284395 |
| SLITRK5 | TEX101 | LINC01159 | LAMP5 | LINC00334 | LINC00971 | THEG5 |
| PSMB11 | APC2 | GUSBP15 | LINC02109 | LRIT1 | C5orf64 | CCL25 |
| LOC100505716 | LOC101927870 | PACRG-AS2 | ZBTB8A | CDH4 | PSD2 | CHST6 |
| UTS2 | EML2-AS1 | B3GAT1 | BPI | MEGF9 | LSAMP-AS1 | LOC105374454 |
| DNAH17-AS1 | SNAR-C3 | PRR36 | ERBB4 | DGCR9 | ZFHX2 | ATCAY |
| LINC01978 | PLA2G4C-AS1 | FAR2P1 | ZSCAN21 | POM121L4P | IGSF11-AS1 | LINC01121 |
| LOC101928855 | RPS6KA1 | LINC01120 | TRPV3 | C19orf18 | LRRC1 | TRIM64B |
| PDHA2 | GRM3 | NCKAP5-AS1 | LINC01733 | VAX1 | IDUA | HAPLN2 |
| EPB41L3 | ZNF610 | PRKCG | LOC100134868 | FAM71C | PRSS54 | LRFN1 |
| LINC01254 | ACTL7A | CDH9 | NOL4L-DT | PVRIG | SERTM2 | LOC100129175 |
| KCNS3 | SHANK1 | DTHD1 | BPIFA4P | IL1RAPL1 | ASTN2 | CFAP65 |
| C2CD4C | TMEM178A | GRIK4 | IPO13 | LOC100506271 | TMEM108 | KRT83 |
| DNAH17 | SPEM1 | CSMD3 | C6orf118 | EBF4 | TNFSF9 | PRKCB |
| KLRC2 | FCHO1 | SYT14 | DPYSL4 | CNTNAP2-AS1 | CERCAM | MYH14 |
| WBP11P1 | LOC100128398 | C10orf120 | FEZ1 | TMEM132C | HEPACAM | LINC01749 |
| KC6 | A1BG-AS1 | AHCTF1P1 | BPIFC | ELOVL6 | DOHH | LINC00029 |
| BBS2 | LINC01250 | ACP7 | MKRN7P | LOC105373051 | GABRG3 | LINC01056 |
| LINC01416 | SILC1 | ZNF713 | NIPA1 | LINC01310 | SLC7A14-AS1 | FLJ16779 |
| CRB2 | LOC400940 | TTBK1 | F11-AS1 | LINC01266 | UCN | DOCK3 |
| LERFS | ADGRB1 | FAM167A | FAR1 | LOC101927394 | LOC101928882 | SUN5 |
| LINC01922 | GACAT3 | SPO11 | LINC01524 | SPATA19 | LINC01206 | WDR62 |
| LINC01898 | DNAJC27-AS1 | RAB40B | SLC8A2 | LINC00606 | LINC00888 | GLB1L2 |
| LINC01029 | LINC01381 | LINC01923 | BMP7-AS1 | SLC6A1-AS1 | YEATS2-AS1 | DCC |
| LRRC31 | IGDCC3 | LINC01877 | CNMD | PRDM16 | SLCO1A2 | GPR142 |
| PCDHA6 | IAPP | DMRTA2 | MIR646HG | GPR12 | TUBB8 | LOC105374338 |
| GPR75 | KCNK12 | VWC2L | LOC100128310 | LRRC4B | LINC02012 | FAM53A |
| SDS | SALL1 | ENO4 | LOC100506937 | PTMAP11 | LINC02789 | GUSBP3 |
| LINC02495 | CAVIN3 | SCD5 | CNTNAP2 | GAS2L1P2 | DIPK1C | LOC101929599 |
| CEP170P1 | LOC729080 | SYT11 | SRRM5 | ARFGEF3 | CTAGE6 | LOC441081 |
| EMC10 | ELOA3D | PMP2 | LOC100286906 | KIAA1958 | SYT9 | CYP27A1 |
| PTPRH | MFAP3L | KCNK9 | LOC285889 | LINC00858 | FXYD4 | PMCHL2 |
| AJM1 | LINC01470 | HECTD4 | LOC101927914 | TUSC7 | H3Y2 | CRSP8P |
| SEPTIN8 | PADI2 | ZNF648 | LOC286083 | CRYGA | CACNG7 | LOC100133050 |
| YEATS2 | LINC01411 | GPR179 | LOC101927815 | GLT6D1 | SMOC1 | CINP |
| SRD5A3-AS1 | CDR2L | ZNF80 | LINC00599 | LOC101928797 | BPIFB1 | GPRC5D |
| IGFBP7-AS1 | LINC00639 | ATP9B | LOC100129129 | HTRA1 | BTBD17 | NPY6R |
| FLRT1 | TIE1 | MYBPHL | LOC649352 | CLVS2 | OR13A1 | PACRG-AS1 |
| SIX3 | NANOS2 | ENDOD1 | TRPM6 | SHC2 | QRFPR | KIF25-AS1 |
| TPPP | FABP1 | MLANA | CLRN2 | RNFT2 | LMTK3 | LOC285804 |
| LOC284412 | NDE1 | OTOR | LINC00511 | TEX44 | SHISA2 | LINC01624 |
| SEC11C | BPIFB4 | C20orf203 | SGTA | BPIFB2 | ZNF835 | COLGALT2 |
| PCDHA8 | NPBWR2 | KRT81 | LOC101929550 | OXCT2 | VHLL | FBXL16 |
| DIPK2B | FRMPD1 | GPR87 | PCDHA12 | LOC101928786 | ARSI | FLJ40288 |
| RASGEF1C | LARP6 | LRRC72 | SBF1P1 | PPP1R2B | C2orf27B | MYBPH |
| PCDHA7 | ARHGAP33 | TFAMP1 | TUBB8B | TMEM225B | LOC401478 | LHFPL3-AS1 |
| ZNF775 | LOC100144595 | CLK2P1 | LINC00588 | RUNDC3A | ODF3L2 | GMCL2 |
| LOC101927359 | LOC101928936 | WFDC2 | NKAIN3-IT1 | EDIL3 | KRT74 | DOCK4-AS1 |
| LINC02511 | BRINP3 | TOMM20L | LINC01299 | ZNF705A | ZNRF4 | LINC02476 |
| ZIM3 | PNLIPRP1 | ZNF653 | KCNG1 | COL20A1 | ELOVL2 | CEP72 |
| ASPHD1 | PNKY | HECW1-IT1 | REXO1L2P | HCN2 | B4GALNT4 | LOC105375483 |
| NPHS1 | BVES-AS1 | FAM71A | CLEC19A | ATP13A5 | BIRC8 | LINC01189 |
| ANXA2P1 | OSTM1-AS1 | RPL13AP17 | NSG2 | FRMD8P1 | FFAR1 | LOC389765 |
| HMGB4 | TRAPPC2B | GJC2 | BCAN | OCM | PABPC3 | C9orf153 |
| GK3P | HDAC2-AS2 | PGBD5 | LINC01591 | PABPC1L2B- | EEF1A2 | TRIM43B |
| | | | | AS1 | | |
| SPOCK3 | LOC149373 | HID1 | SUGT1P1 | KCNMB4 | SOX21-AS1 | LOC158435 |
| LINC01098 | NKAIN2 | HDAC4 | SEPTIN14 | ZFHX4 | NCAN | APLP1 |
| GUSBP1 | HMGA1P7 | KLHL40 | FA2H | LOC101928336 | LOC100134391 | IL12B |
| LINC02109 | NRON | PNLIPRP3 | FGF7P3 | RHOXF2 | GLRA4 | LOC102724995 |
| LAMC3 | OC105371031 | C17orf102 | SLC24A2 | RAB6C-AS1 | LRRC74A | LG13 |
| ANGPTL7 | SLC7A14 | ZNF561-AS1 | | | | |

Breast

| | | | | | | |
|---|---|---|---|---|---|---|
| OR1K1 | MUC1 | ALPL | NTN3 | PCDH19 | LOC729296 | ICAM4 |
| SFRP2 | PRRX2 | MXRA5 | ST8SIA2 | NEUROG1 | LAGE3 | RBP5 |
| LINC01777 | NEBL-AS1 | NTM-AS1 | PIK3R6 | KLK1 | SLC7A3 | JSRP1 |
| C1orf159 | DOCK2 | C6orf15 | NRSN1 | PTGER1 | ARX | SPAAR |
| WNT11 | FOXI1 | TNNT3 | LINC00968 | FMO6P | LOC110091777 | CROCC2 |
| SHOX2 | AHDC1 | SUSD2 | THEG | KRTAP10-2 | TNFRSF18 | LINC00589 |
| LINC01141 | LOC105378269 | APLNR | RXFP3 | SMIM33 | PIK3R5 | GPR42 |
| RGMA | LHX6 | PRR20G | SMIM10L2A | CPXM1 | LOC101928126 | ODF1 |
| PHLDA2 | C10orf71-AS1 | FOXD3-AS1 | STMN4 | GPR88 | LINC01426 | KRT5 |
| PEBP4 | USP50 | SOX8 | LINC02204 | KCNA1 | IZUMO1R | LOC101928674 |
| MEOX1 | TSHZ2 | TBX1 | WNT6 | SLC17A7 | LINC00323 | LINC01443 |
| IL17RD | TAZ | GRASP-AS1 | LOC283731 | ID2-AS1 | GSC | GATA6-AS1 |
| BHLHA9 | GJD4 | CISTR | HOXA11-AS | EBF2 | COL18A1-AS1 | WNT10A |
| S100A1 | WNT10B | HOXC13-AS | WAKMAR2 | SIX3-AS1 | BCRP2 | LINC02285 |
| LINC01763 | MAP7D2 | HOXC-AS2 | RNF186 | LOC100507006 | RNF225 | LINC02314 |
| HAUS7 | LINC02622 | LOC101927583 | LOC105369266 | AKAP3 | HOXD3 | LINC00605 |
| APLN | PEAR1 | ADAMTS12 | HAGLR | TMEM59L | GUCY2D | TMEM273 |
| MAGEA4 | AGAP2-AS1 | RNF165 | MMP2-AS1 | FOXL2NB | COL14A1 | SP6 |
| PRSS21 | PRSS55 | LINC02401 | STAC2 | LINC01964 | FSCN2 | NRN1 |
| ISLR2 | RTL5 | RBP4 | MIR3183 | CRLF1 | KLK5 | FAM181B |
| TMEM151B | HS6ST2 | LINC00540 | RIN1 | PRRG3 | SOST | EDN3 |
| FBLN7 | FANK1 | ROR2 | KRT17P1 | COL5A3 | LINC00696 | APCDD1L |
| LMX1B | LINC01150 | MAGEL2 | KRT17P2 | TEX33 | ESRG | HHIPL1 |
| BARHL2 | KCNQ1DN | OPN1LW | C16orf82 | LYL1 | FOXC2 | HOXC9 |
| YPEL4 | IL11 | KRT6B | LRRN2 | CYP11B2 | COL2A1 | LINC01744 |
| HLA-G | CALHM3 | OLIG3 | CRABP2 | SBK2 | TMEM200C | UCMA |
| BOC | CHRD | ARHGEF7- | AWAT2 | ADGRF1 | LINC02044 | MIR200B |
| | | AS2 | | | | |
| PTGES | TTLL10 | EGR3 | KCNB2 | HOXC10 | TBX2 | LGALS7B |
| KCNAB2 | FAM180B | ICAM5 | PLET1 | MAGEA3 | TRIM72 | TMEM119 |
| CHRDL1 | KRT71 | PLXNA1 | RTL1 | DERL3 | LGALS7 | DBET |
| MMP13 | PHF7 | NKX1-2 | LINC02075 | SPATA31A3 | SPTSSB | EGFLAM-AS4 |
| PLAU | MYH13 | TREX2 | RSPO4 | PGM5-AS1 | PADI3 | ANO1 |
| GJD3 | SRPX | SPATA32 | TSPOAP1-AS1 | ALX4 | LINC01396 | SCARF2 |
| SPATA31A1 | GHRH | DMRT3 | PLP1 | LOC101928881 | ADAMTS2 | CLCN1 |
| DUSP13 | LOC338694 | KLK9 | LINC01482 | TMEM239 | INSRR | KRT78 |
| JAG2 | TMEM200B | GSC-DT | LINC02003 | MRC2 | CEROX1 | HOXD1 |
| KLK8 | BTG4 | MEG9 | VPREB3 | IRX3 | IL13 | FBXO2 |
| KRT79 | SPATA31A6 | FIBIN | C1QL4 | NALT1 | LPCAT2 | MYOZ3 |
| TBX18 | SEMA3G | FOXD4 | DBX2 | ETV3L | SIX2 | ARL5C |
| IZUMO3 | LOC101929470 | CALHM1 | EN1 | FAM239B | METTL27 | FZD10 |
| PTGER2 | LINC02692 | OSR1 | SAMD5 | MT3 | TMEM30B | C3orf80 |
| CAV3 | DEGS2 | FMNL1 | GRIFIN | LOC100129291 | KLHL13 | NXPH4 |
| LOC101929122 | CPA1 | PNMA8B | MMP3 | PTPRVP | FOXD2 | DRD2 |
| AGPAT4-IT1 | FNDC10 | KRT15 | FOX12 | KRT6C | FAM43B | LOC105375115 |
| LINC00602 | DDN | PHOX2A | GSDMA | TDGF1P3 | UTS2R | WDR86 |
| LINC02544 | MAFA-AS1 | DLX3 | BICDL2 | KRT6A | CAVIN1 | LOC100506178 |
| DTX1 | PRR35 | TIFAB | TAS2R41 | LCA10 | WDR86-AS1 | C7orf57 |
| LINC02247 | FGF7P6 | LOC401557 | GAL | CGB7 | BARX1-DT | LOC494127 |

Heart

| | | | | | |
|---|---|---|---|---|---|
| CAP2 | TRMT13 | ZNF551 | ADRA1A | WDR37 | ZNF256 |
| SPSB4 | LRRC39 | NDRG4 | JPH1 | TTN | EXTL3 |
| PCDHGC5 | TUBA8 | LRRC37A6P | ZNF232 | SPEG | PNMA3 |
| MOG | PFKM | USP28 | SBK3 | PLXNA4 | KCNH2 |
| EXOG | PPP1R17 | ZNF213 | HRH2 | NEBL | SPEGNB |
| LY86 | AKR7A2P1 | RPUSD4 | JPH2 | MYOCD | LOC105370361 |
| ATG4A | ZSCAN16 | ZNF772 | CACNB2 | NUTM2D | SCN5A |
| UQCR11 | NGF-AS1 | GNRH1 | ARHGAP29 | MYL2 | GIMAP8 |
| TANGO2 | CHRM2 | SMYD1 | PAX6-AS1 | MYPN | MCF2L-AS1 |
| ANK2 | TTN-AS1 | CACNA1C | ADAM23 | SLC20A2 | MIR8075 |
| MEF2C | PRNP | LINC02664 | MSN | DNAJB6 | LRRN4 |
| RDH13 | OXTR | ARHGAP22-IT1 | NKX6-3 | PCDHGB2 | ABCC9 |
| CD36 | SMIM27 | HCLS1 | TRIM54 | SLC8A1 | CDCA7L |
| ACTG1P20 | PCDHGB3 | SLC16A7 | FAM181A | JAM2 | PPP1R3A |
| PDE1C | DMD | CLEC2D | FASTKD5 | DPY19L2 | SLC5A1 |
| AKAP6 | DISC2 | TMEM71 | BRINP2 | LOC101928002 | IGSF9B |
| NRP1 | HCN4 | LRRTM3 | PCDHGA2 | PRDM16-DT | LINC02322 |
| STOX2 | TNNT1 | CDH13 | ARL11 | MRS2P2 | LOC100506321 |
| CASP9 | UNC45B | RASGRP3 | GPRC5B | CCDC59 | CAMK2B |
| ANKS4B | OR51E2 | KRBA2 | LINC01488 | PCDHGA3 | SEMA6C |
| PPP1R13L | ARHGAP42 | NMRK2 | CMYA5 | TUBE1 | DDX60 |
| BZW2 | TMEM106C | RAD51AP1 | MIEF2 | CASC18 | NAP1L5 |
| COX411 | MYOT | MCRS1 | NCAM1 | LOC105369980 | CCDC197 |
| CLLU1 | HIF3A | NEURL1B | PPP2R3B | ARHGAP10 | PCDHGA8 |
| HDHD5 | LINC01732 | SLIT1-AS1 | MITF | TBX5-AS1 | RGS6 |
| GDAP1L1 | PAM | ASTN1 | PCDHGA4 | CCDC70 | GJA3 |
| CACNG1 | TSPAN9 | GYPC | NCAM1-AS1 | MTRNR2L8 | OTOG |
| MIXL1 | ZNF548 | CFAP61 | AFAP1L1 | MTUS2-AS1 | PALMD |
| SKINT1L | DNAJC5G | CDH2 | FHOD3 | PCDH9 | SPHKAP |
| SERPINB4 | WDR60 | COL4A1 | MLEC | CA1 | IDH3A |
| TBX5 | PCDHGB7 | NUTM2A | ZNF550 | FRY-AS1 | DPH6-DT |
| FLNC | PPARGC1A | LINC02620 | LOC341056 | DAPK2 | CSNK1A1P1 |
| PPFIBP1 | ZNF112 | CSRP3 | C10orf71 | LINC01050 | IDI2 |
| DPF3 | EPHA4 | LOC101927692 | ZNF304 | LINC00390 | CFAP20 |
| DET1 | NIF3L1 | MYO18B | TENM2 | OBSCN | GINS3 |
| GSPT2 | PSD3 | FITM2 | MIR4697HG | CLIC5 | FBXL7 |
| ABCB7 | LOC101930421 | PKIA | LOC105369595 | LINC02338 | FGF12 |
| LRRC53 | LINC00703 | RBFOX1 | LINC00940 | CCT2 | PIK3R6 |
| COX6B1 | LOC101928841 | RWDD3 | CACNA1C-AS4 | ITGA7 | MGST3 |
| PCDHGA12 | MAGI1 | TRIM55 | A2M-AS1 | LINC00382 | ZNF17 |
| PCDHGB6 | CELF2-AS2 | MLF1 | MLIP | FSD2 | TNNT2 |
| PCDHGB5 | CELF2-AS1 | LCN10 | TMEM40 | KLHL7 | PWAR6 |
| CD8A | CORO6 | LINC02560 | ZNF462 | UCN | MYH6 |
| GOLGA2P10 | ASB8 | FBXO40 | MIR1-1HG-AS1 | RYR2 | E2F8 |
| DNAAF3 | MPP3 | LINC01954 | KRT32 | GHITM | THSD4-AS1 |
| ZNF805 | SCN10A | PCDHGA7 | ZBTB46-AS1 | LINC00888 | KCNMB2 |
| LINC01672 | METTL7B | CENPA | TRH | ADIPOQ-AS1 | TYRO3P |
| LINC00930 | MYADML2 | ATCAY | CEP192 | FGF12-AS2 | PTPRM |
| LINC00923 | LRRC2 | DDI1 | GNAT3 | MGC2889 | MAPK4 |
| GALNT17 | CDH5 | RPL23AP32 | LOC101928398 | SDHAP2 | LOC101927189 |
| MIR3176 | PGAP4 | CCDC85A | SYNM | UQCRC2 | GRAMD4 |
| ATP1A2 | ZNF638 | TOMM7 | LRRC3-DT | LINC01182 | LOC101928540 |
| CUL4A | LPAR3 | FBXO38 | LOC101928796 | PLCL1 | POPDC3 |
| RAB12 | LINC02563 | MEX3B | SNRPC | LOC101929577 | GPR52 |
| GOT2 | MTUS2 | DLC1 | SLCO3A1 | PCDHGA11 | LIN37 |
| HOMER1 | LINC02092 | ATP5MPL | LINC00207 | PCDHGA9 | CAV3 |
| NUTM2E | MYOM3 | PCDHGA1 | FAM3D-AS1 | LOC101929762 | TRDN-AS1 |
| ASB14 | NDUFB8 | STARD7-AS1 | XIRP2 | KLHL34 | CPTP |
| DNAI2 | NRAP | FILIP1 | PRDM16 | ADAL | ECT2L |
| IRX4 | LOC100192426 | PRRG3 | TECRL | BCAR3-AS1 | PACRG-AS3 |
| PXDNL | C18orf15 | LINC02145 | PLCL2 | PCOLCE2 | DKFZp451B082 |
| ZNF843 | NDUFA8 | GJC1 | DIRAS1 | LOC100506688 | MRPS30 |
| ESRRB | GUCA2A | LARP7 | GABRA4 | MRPS6 | MAD2L1BP |
| SLC6A10P | LCLAT1 | DSC2 | TSSK1B | LINC02112 | NDUFB9 |
| PTTG2 | LINC01630 | HADH | WDR62 | LINC02150 | GABRP |
| LINC02140 | NINJ2 | GAB3 | KBTBD13 | TMEM86A | ABCC12 |
| ARHGAP31 | APOBEC4 | C3orf33 | KCNK4-TEX40 | LOC100506639 | FETUB |
| KCNA5 | MAST2 | SRL | LINC02009 | LOC101928505 | REC114 |
| ABLIM3 | ADRA2B | MRPL39 | PRSS42P | LINC02101 | NUTM2B |
| MAB21L1 | A2M | ATP1A3 | HECA | LOC101929599 | CRPPA-AS1 |
| CHRNA6 | KRTAP10-8 | KCND3 | ACTN2 | LOC441081 | LINC00997 |
| SGCD | GALNT8 | PFDN4 | LNCAROD | C6orf163 | DPY19L2P1 |
| WT1-AS | SEC14L5 | PCDHGB4 | GRPEL2 | LINC02208 | LOC401324 |
| DAPK3 | KCNJ12 | ZNF71 | MOGAT2 | LAMA2 | CILP2 |
| LOC101928446 | ZNF154 | LINC00654 | CKMT2 | DUSP27 | VWC2 |
| LOC101928417 | TMEM182 | LINC01722 | PTPRG-AS1 | PCDHGB8P | LINC01372 |
| ZNF417 | ZFPM2 | PCDHGA10 | PCDHGB1 | C5orf15 | FAM200A |
| SLC25A24 | ZNF571-AS1 | ASB15 | SIPA1L2 | GPRIN3 | MB21D2 |
| GBE1 | PCDHGA6 | HUS1B | MRPL27 | LINC02202 | NAV1 |
| SRXN1 | LOC100505715 | TOR4A | ATP5ME | LINC01845 | FGF18 |
| MRPL15 | ZNF671 | CD300LG | DTX3 | CTB-178M22.2 | ZNF597 |
| MYOM2 | KCNJ4 | SH3RF2 | GIMAP6 | LOC100505502 | FADS3 |
| LOC100128006 | NR2E3 | MUL1 | LOC105376731 | ING4 | KLHL30 |
| SMC01 | ZNF586 | ADAMTS15 | CHCHD3 | UQCRC1 | IER2 |
| GOLGA8S | UBE2CP5 | LPL | LRRC77P | FABP4 | FLNC-AS1 |
| LOC100129129 | MYOPARR | BEND2 | NDUFS6 | TAS2R31 | RTRAF |
| LINC00208 | PRSS44P | PCDHB13 | LINC01505 | MIR12136 | CRIP1 |
| LINC00681 | ASB18 | TAS2R13 | SMCO4 | OR10P1 | UTS2R |
| LOC101929237 | SMCO2 | SMPD5 | NDUFV1 | LRRTM1 | NGH |
| ROR1-AS1 | LRRC30 | ATP13A5 | LOC613206 | ADAMTS7 | NDUFA9 |
| CCNB1IP1 | RBM12B | SAMM50 | DSG1 | CCDC127 | MMP15 |
| STK35 | SLC38A8 | IQCF6 | TRAK2 | PCDHGC4 | DCAF4L1 |
| PKIA-AS1 | TAF7 | SWSAP1 | PPARGC1B | UBAC1 | FAM110B |
| FLJ46284 | FAM229B | KRTAP10-9 | LOC101928932 | EEF1A2 | ZNF780B |
| LOC104054148 | PLAC8L1 | MTRNR2L2 | SLC25A53 | KCNA4 | PLCXD3 |
| TMEM38A | SAMD5 | MTRNR2L4 | LINC00685 | LINC01170 | KLHL31 |
| FER1L6-AS1 | DCUN1D2 | KBTBD8 | HEATR5B | CYCS | KRT74 |
| LOC105375773 | NCRNA00250 | PCDHGA5 | SLC22A7 | TESC | ISOC1 |
| TIMM21 | | | | | |

Hypothalamus

| | | | | | |
|---|---|---|---|---|---|
| PPP1R9A | SPATA6 | FAM171B | GRIA2 | MYO16 | MXD1 |
| KIF1A | NOS1 | LRP1B | LOC101929662 | DNAH10 | MEGF10 |
| KLK6 | BRINP1 | ADGRL3 | STOX1 | SPIRE1 | PPP3CB-AS1 |
| LINC01647 | DPP6 | SHTN1 | GRID1-AS1 | SORCS1 | CACNA1E |
| RGR | FBXO15 | MIR4454 | MOBP | ADGRA1 | SYCP2L |
| KAZN-AS1 | PCDHA5 | SLC6A1 | CLCA4 | TMC7 | PAK5 |
| AGAP1 | HAPLN3 | SPECC1 | LMF1 | KIF19 | EIF2S3B |
| C10orf90 | ARPP21 | NRXN1 | TRIM49C | SLC4A8 | ENPP2 |
| ST18 | MAL | LINC00200 | PRSS55 | CACNG4 | CTNND2 |
| PAQR8 | NRXN2 | ADARB2-AS1 | CAVIN4 | SLC5A4 | KCNJ9 |
| LINC01654 | CACNG6 | LINC00700 | SORCS3-AS1 | ZNF565 | HCG22 |
| TIAM1 | EPHB1 | FAM205C | MMP16 | MC4R | PTPRZ1 |
| RIMS2 | PPP1R17 | ZRANB3 | CSRP3 | LSAMP | PNLIP |
| PPP2R2B | CADM1 | LINC02648 | DOCK10 | FUJ42102 | CNTN1 |
| NTRK3 | KCNQ2 | PCDH15 | RYR3 | KCND2 | CECR2 |
| PPFIA2 | SRCIN1 | LRTM2 | GAL3ST1 | ADGRB3 | OBI1-AS1 |
| PPP2R2C | LINC01649 | KIT | PTPRD | MIR4300HG | CAPN9 |
| SLC6A15 | DSCAM | RORB | DMBT1P1 | KRCC1 | UNC93A |
| NFASC | LOC101927468 | ELMOD1 | PINX1 | ADARB2 | PRKCQ |
| TMTC4 | AGA | ARMC3 | LINC01166 | NCAM1 | MIR124-2HG |
| SLC26A5 | ABCA12 | NCAM2 | ADGRA1-AS1 | SALL3 | MMD2 |
| ZNF2 | EFNA2 | CDH19 | CFAP161 | FAM135B | INPP5A |
| SLC39A11 | BARHL2 | TUBB4A | DNAJC12 | IZUMO2 | HHATL |
| C4orf50 | WFIKKN2 | NENF | GRIN2A | SLC10A2 | SCN3A |
| LRRTM4 | GRIK2 | GPR15 | ADD2 | SCEL | NRG3 |
| AKAP6 | SLC5A11 | GRIA4 | LINC00466 | GLTP | FUT8 |
| MYT1L | ELAVL4 | KIF3A | KCNJ10 | CDHR1 | BRI3BP |
| LUZP2 | INSYN2A | LINGO1 | WDYHV1 | PHACTR3 | LRIT2 |
| CSMD2-AS1 | C12orf56 | RSU1P2 | OTX2 | HMX1 | TULP4 |
| CTNNA2 | GABRB3 | SH3GL3 | DSCAML1 | GTF2F2 | TRIM64 |
| ANLN | PLD5 | CNDP1 | CNTNAP4 | WASF3 | C22orf34 |
| RGS7 | APBA2 | GPM6A | RPH3A | KCNH1 | TAFA5 |
| OXCT2P1 | LINC01657 | FRMPD2 | MARCHF1 | SYNDIG1 | NPIPB15 |
| SATB2 | TMEM145 | SEC61A2 | SLC16A14 | IFIT1B | PDE6B |
| KLHDC4 | CTNNA3 | RTN1 | RBP3 | PANTR1 | NHLH2 |
| SEZ6L | DGKI | ILDR2 | FUT9 | GOLGA6L7 | CDH10 |
| CLEC4A | SCN1A | NPAS3 | AP3B2 | LOC101929653 | TMEM132B |
| ZNF415 | NRXN3 | PTPN11 | NCKAP5 | NTM | LINC00943 |
| KIAA1324L | NRF1 | DNTT | PAUPAR | LOC283177 | LINC02393 |
| FSTL5 | ASB4 | RPS6KA2 | ADAM23 | KLK7 | TMEM132D-AS2 |
| ACTRT2 | SYT3 | DNAJC22 | COL11A1 | HIVEP3 | LINC02415 |
| ZDHHC22 | CLEC4F | KCNK10 | ASPA | TMEM161B | GRK6P1 |
| LOC101927248 | LINC00928 | DNAH17-AS1 | GOLGA8M | LOC101928107 | LINC00463 |
| DPYSL5 | KLRC3 | LOC101928855 | TRIM64B | TIGD4 | LINC01053 |
| FAM155A-IT1 | NPSR1 | DLGAP1-AS5 | DSCAM-AS1 | SMOX | LOC101929259 |
| LOC105375131 | TMEM151A | LINC01287 | CADM2 | CDH4 | PTN |
| LINC00354 | ATP13A4 | LINC01543 | CLRN1 | GRXCR2 | ATP10B |
| SOX1-OT | LINC01567 | DNAH17 | LINC01102 | POM121L4P | LINC02026 |
| LOC100506016 | GAD1 | WBP11P1 | LINC01103 | CFAP73 | LINC02012 |
| GRID2 | DNAI2 | SCRG1 | LINC01114 | IL1RAPL1 | LOC105374338 |
| ST6GALNAC3 | KIAA1549 | LHCGR | LINC01159 | LOC100506271 | LINC02495 |
| JAKMIP2 | CALN1 | BBS2 | GUSBP15 | ELOVL6 | CEP170P1 |
| GPR45 | TRIM49 | NTRK2 | B3GAT1 | LINC01310 | SHISA9 |
| HS6ST3 | VANGL2 | LINC01630 | EFCAB6 | MRPS9 | YEATS2 |
| AJAP1 | CNR1 | GOLGA8J | DTHD1 | LOC100130207 | RASGEF1C |
| VSTM2B | SLC22A6 | OACYLP | CSMD3 | LOC101927394 | PNMA2 |
| MDGA2 | LINC01568 | LINC01922 | SYT14 | SPATA19 | LOC101927359 |
| COL22A1 | LINC02125 | LINC01029 | DUSP18 | LINC00606 | LINC02511 |
| SUMO1P3 | LOC339166 | GPR75 | ZNF713 | GPR12 | SCOC-AS1 |
| WASHC5 | AGTPBP1 | RELN | FAM167A | LRRC4B | COL9A1 |
| LRP12 | ELOA2 | IL12RB1 | SPO11 | SUN5 | ASPHD1 |
| GRID1 | PCDH20 | GALNT8 | LRRC4C | GLB1L2 | ANXA2P1 |
| CLUL1 | IGLON5 | LOC284395 | VWC2L | DCC | HMGB4 |
| PSAT1 | NOS2P3 | CHST6 | SLC7A14 | GPR142 | SPOCK3 |
| USP17L4 | LOC100287072 | GNAZ | KRT83 | WSCD1 | ERVH48-1 |
| LINC00642 | PPY2P | PLEKHH1 | REELD1 | LINC01520 | LINC01098 |
| UNC79 | CDH6 | CDH20 | PRKCB | CST1 | LOC100506858 |
| SERPINA2 | CDH12 | LINC00320 | NLGN4X | HAPLN1 | LINC02112 |
| PDCD6IPP2 | CCDC62 | APC2 | CSMD1 | MDFIC2 | GUSBP1 |
| AK5 | CPPED1 | SNAR-C3 | LINC00658 | SHISA7 | C5orf64 |
| LOC646652 | LRRC8B | TMEM231 | LAMP5-AS1 | PSD2 | LOC101928651 |
| LOC105370757 | RUNDC3A-AS1 | GRM3 | LAMP5 | DUBR | GUSBP3 |
| TMCO5B | NALCN | SHANK1 | KIZ-AS1 | LSAMP-AS1 | LOC340090 |
| WDR47 | CLVS1 | GPR158 | ERBB4 | ZFHX2 | RANBP17 |
| FAM189A1 | LINC02072 | MROH9 | LINC01733 | PRSS54 | PREX2 |
| CBLN2 | LYZL1 | LINC01250 | LOC100134868 | ASTN2 | CINP |
| RASGRF2 | IL3 | SILC1 | C6orf118 | TMEM108 | LOC101927421 |
| SYT16 | FAM171A1 | LOC400940 | KCNH7 | HEPACAM | SNHG4 |
| STMN4 | ERICH2 | GACAT3 | FEZ1 | TIAF1 | GRIK3 |
| JAKMIP3 | KCNH8 | DNAJC27-AS1 | NIPA1 | LDHAL6B | GPR32 |
| ZNF536 | LOC101928251 | IAPP | LOC100128310 | LINC01206 | LOC729080 |
| LINGO1-AS1 | JAKMIP1 | LOC100505716 | LINC01056 | LINC01994 | STK32A-AS1 |
| LINGO1-AS2 | RNF125 | DISP3 | DOCK3 | YEATS2-AS1 | JAKMIP2-AS1 |
| PTPRN2 | SLITRK5 | LOC105374454 | BPIFB3 | LINC02052 | MFAP3L |
| NTRK3-AS1 | PSMB11 | SIX3-AS1 | CDH8 | SLCO1A2 | LINC01187 |
| TRIM43B | RPS6KA2-IT1 | SYT11 | KIAA0087 | BPIFB4 | PADI2 |
| LOC340512 | COLGALT2 | PMP2 | RPL13AP17 | NPBWR2 | LINC00639 |
| RNF182 | CTTNBP2 | ZNF648 | PGBD5 | FRMPD1 | LINC00599 |
| LINC00858 | CCDC175 | GPR179 | LHFPL3-AS1 | BRINP3 | TRPM6 |
| TUSC7 | DSPP | CER1 | LOC105375483 | LOC101927314 | CLRN2 |
| LOC101928797 | MAP2 | ATP9B | POT1-AS1 | PNKY | ZFHX4-AS1 |
| SUDS3 | SLC24A2 | CLEC1B | CNTNAP2 | LOC149373 | NSG2 |
| TEX44 | NANOS2 | ENDOD1 | SRRM5 | NKAIN2 | BCAN |
| BPIFB2 | FABP1 | GPR87 | LOC100286906 | HMGA1P7 | RGS9 |
| OXCT2 | NDE1 | TFAMP1 | LOC101927815 | PACRG-AS2 | C9orf62 |
| LOC101928786 | CA5BP1-CA5B | ZFHX4 | PABPC3 | ZNRF4 | BTBD17 |
| PPP1R2B | LOC102724995 | MOB3B | RFTN2 | ATP13A5 | SLC38A8 |
| RUNDC3A | DIPK1C | RHOXF2 | SOX21-AS1 | PABPC1L2B-AS1 | QRFPR |
| EDIL3 | SYT9 | APLP1 | LRRC74A | ZNF835 | SHISA2 |
| BPIFB1 | CACNG7 | FAM210B | IL12B | | |

Kidney

| | | | | | |
|---|---|---|---|---|---|
| LINC01342 | SLC25A10 | ATP6V1B2 | FAM245A | RIPK4 | FXYD6 |
| STOML3 | LYG1 | KHK | SGK2 | HCN3 | CDH16 |
| WDR18 | SLC36A2 | LINC01116 | RAB35 | GJA4 | GAS8-AS1 |
| CES3 | SETD3 | RFWD3 | INPP5J | TUBAL3 | TMEM117 |
| TNFRSF14-AS1 | TMPRSS4 | GGT2 | RRAD | SLC26A6 | WWC1 |
| ADAMTS9 | LINC01761 | SFTA2 | MORN4 | TMEM101 | LLGL2 |
| NDUFS7 | TMPRSS2 | MISP | CFAP61 | PALM | TMEM213 |
| NEURL2 | GRM2 | TRMU | ASAP2 | GLIS2 | PAX8-AS1 |
| C1orf195 | ANKZF1 | HOXC9 | IFIT3 | FGFR4 | KCNE3 |
| KLHDC7A | MAL | PPARGC1A | TTLL6 | SHANK2-AS1 | FLJ12825 |
| ARHGEF10L | SEMA4A | GALNT14 | CASR | SHANK2-AS3 | LOC100240735 |
| FCAMR | C12orf43 | GPAT3 | SH3TC1 | MPC1L | LACTBL1 |
| KNG1 | CEBPA | ADGRG1 | MRI1 | EIF4E2 | BICDL1 |
| KCNJ16 | LDAH | LOC101930421 | DGCR5 | HOXA1 | ST14 |
| IYD | PLAAT4 | MYO15B | GAL3ST1 | STX16 | G6PC |
| SLC13A3 | OGDHL | HIP1R | ACOT11 | SLC7A8 | TINAG |
| ANKRD33 | POU5F1 | PALM3 | EDRF1-DT | TSPAN33 | SORCS3 |
| ESPN | ATP1A1-AS1 | CGNL1 | TMEM171 | CYFIP2 | PTGER3 |
| ZNF488 | CWH43 | FOXN1 | LINC01167 | NAA60 | GDF7 |
| NELL1 | SLC22A13 | POMGNT1 | CYP8B1 | FBXO34 | PGAM1P5 |
| TNFRSF21 | PAX2 | CA12 | CDPF1 | CRYL1 | ZNF697 |
| PROZ | FGFR3 | MME | SFXN5 | RGL4 | GOLGA2P5 |
| CLCN5 | IQSEC3 | AIF1L | TRPC2 | TNFRSF11B | UPP2 |
| TP53AIP1 | GALNT9 | STON2 | PIGQ | LOC101929340 | KCCAT198 |
| HNF1A | BEND5 | USH1C | LOC283299 | LOC100507283 | TRPV4 |
| CTCFL | AGPAT2 | LINC01518 | DPPA2 | DTNBP1 | C9orf116 |
| GPRC5C | CHMP1A | RASGRP1 | SPAG7 | TBC1D7 | PRR5 |
| ZBED2 | SNX29 | CTSLP2 | GGTLC1 | DPEP1 | FBXO17 |
| SNORA110 | WDR54 | SLC5A9 | GPR31 | LOC574538 | IGLL1 |
| RAP1GAP | SLC17A3 | FAM133CP | SLC22A24 | LOC101929384 | LINC02361 |
| PAX8 | C12orf65 | SLC16A9 | VTN | SLC6A13 | LOC100130238 |
| PPP1R16A | TOP1P1 | CARD10 | CIT | PDSS1 | AMFR |
| IDNK | SMIM21 | SCTR | SLC5A10 | DSTNP2 | TOX3 |
| ZNF618 | AMBP | LINC02671 | DSCAML1 | SLC7A9 | NINL |
| TRPM3 | TRPC7 | DNAJC22 | CD86 | HMOX2 | BASP1P1 |
| SLC25A3P1 | LINC01350 | ADAMTS16 | RBM47 | MPST | SLC6A12 |
| HSD52 | PLG | HNRNPCL2 | CLDN10 | KRT18 | CA4 |
| HOXC4 | HNF1B | LOC101928994 | SKA3 | RNF152 | SELENOM |
| FRAS1 | DAB2 | RAC3 | TMEM51 | ARHGEF28 | OVOL1 |
| RIPOR3 | PANK1 | THSD7A | EVC | SLC13A2 | ITGB2 |
| MIR425 | ILDR1 | EPB41L1 | C9orf57 | SLC22A8 | SLC25A30 |
| MUC20 | ERLNC1 | ZP2 | NANP | FCGBP | KLF15 |
| ATP6V1B1 | TRIM10 | ELAVL1 | UNC5CL | SPP1 | SCT |
| BCAM | LOC652276 | FAM215A | ABO | FAM131C | FAM3D-AS1 |
| C10orf62 | BSND | FLRT3 | SLC17A4 | PCK1 | SOSTDC1 |
| LINC00410 | ACOX2 | MAPT-IT1 | MIR200A | PCGEM1 | DNMT3L-AS1 |
| CLYBL-AS2 | LINC00689 | KRT85 | HOXB1 | STPG3 | HSPA12A |
| GGACT | WDR72 | CUBN | LINC00906 | CNPPD1 | KIF12 |
| ARHGEF7-AS2 | GPX2 | LTB4R2 | LINC01532 | SOX9 | MIR126 |
| MCF2L-AS1 | LRRC75B | RSAD1 | FADD | FIGNL2 | DAO |
| THBS1 | ISM2 | PGAP4 | LINC00665 | ELOA3 | FGF9 |
| GHDC | LOC100128079 | KAZN | FAM20C | LOC100286922 | CST5 |
| SLC22A12 | CLUHP3 | PARD6B | DDC | AGAP1-IT1 | CHCHD4 |
| NDUFB2 | CASC16 | DRD5 | ACVR1B | MYBBP1A | SULT1C2 |
| SLC34A1 | PLA2G15 | NBL1 | RAG1 | MRPS12 | LINC00488 |
| RPL13AP3 | XKR4 | CYSRT1 | ACSM2A | DNMT3L | UGT1A1 |
| AVPR1B | CMTM3 | LINC01561 | LINC01872 | LZTS3 | RNF183 |
| LOC101928123 | MMP24 | LINC01497 | KCNK1 | MIOX | NUAK2 |
| TEX51 | EVPL | LINC01028 | PKHD1 | FUT5 | SULT4A1 |
| OXR1 | SKAP2 | KCNJ15 | ACSM2B | BSG | AP1S1 |
| CPNE6 | LINC02745 | NRTN | LMNTD2 | KIAA1671-AS1 | ADRA2C |
| HOXC11 | RGL3 | SLC25A48 | TRIM15 | TMEM176A | FAM83C |
| PWAR5 | SLC22A6 | APOBEC2 | LINC00570 | ZBTB46 | TMEM37 |
| COQ9 | LRP2 | GPER1 | HOXC5 | STX3 | HSPB9 |
| LOC100130111 | AKAP4 | IGSF3 | CYP4A11 | ADAMTS15 | FAM106C |
| TEX52 | LOC100506281 | SLC4A11 | SLC47A1 | F11-AS1 | AGXT2 |
| CERNA2 | RNF133 | LOC727896 | SLC22A2 | NKAIN4 | CGN |
| CCDC198 | SHH | TOPAZ1 | FOXH1 | PLXNB1 | HOTAIR |
| KCNH6 | EPHX2 | NYX | RAB11FIP3 | SIAH3 | SLC9A3 |
| PAQR5 | LINC01996 | BHMT2 | THNSL2 | AMN | HAVCR2 |
| LOC145694 | ALDH4A1 | TMEM132E | GGT8P | NRAD1 | LINC02038 |
| DRAIC | ELOA2 | SNHG22 | NEURL3 | LINC00112 | LINC00887 |
| CELF6 | DNASE1L2 | CARMIL3 | SLC25A47 | WNT9B | XXYLT1-AS1 |
| PPY | ARPC4 | ANO9 | LOC107105282 | GGT3P | ALDOB |
| AHSG | ZSCAN18 | CLEC18A | PRR36 | FBXW4P1 | LINC00955 |
| SLCO4C1 | DNAJC11 | SLC3A1 | PIGR | ZNRF3-AS1 | CHAD |
| CARD19 | PYY2 | LINC01544 | RMST | MYL3 | SHISA9 |
| NPR3 | CDH6 | GBA3 | MARCHF9 | C16orf58 | IRS1 |
| NR2F2-AS1 | GGT6 | PTH1R | SLC4A1 | LINC02558 | ELF3 |
| KLHL25 | SLC23A1 | LINC00908 | AOC1 | ISX-AS1 | FLRT1 |
| HSP90B2P | MAB21L2 | PDZD3 | LOC401021 | FAT1 | PCSK1N |
| LOC100287175 | ATP4B | LRRC19 | HELT | LOC100506271 | OPLAH |
| LINC02714 | LHX1-DT | METRN | DERA | SULT1C4 | CTSV |
| GRB7 | YWHAEP7 | LOC101928844 | ERBB2 | MPPED2 | PLCL1 |
| THY1 | AOC4P | PRODH2 | METTL1 | KCTD6 | EFHD1 |
| LINC01095 | LINC00671 | HAVCR1 | GAL3ST2 | CRNN | WSPAR |
| CD83 | FOXJ1 | DMGDH | FAM151A | EMX1 | RHPN2 |
| LOC729558 | TPI1P3 | ARHGEF34P | MRO | LOC105374378 | DDC-AS1 |
| LINC01179 | KCNV2 | LOC728743 | RNF113B | BBOX1 | PMS2P4 |
| LINC02275 | RPS18P9 | SPAG5 | PAPLN | SYNDIG1L | SPATA24 |
| FAM92A1P2 | NTN4 | LOC101928058 | C1QTNF8 | ANGPTL8 | FMC1 |
| SLED1 | ABHD14A | PPP1R26 | CLEC18C | FAM83G | SLC6A18 |
| MRPS33 | BCL7A | SMTNL2 | NAGS | SDC4 | GGT1 |
| SLC9A3-AS1 | SMIM24 | APEH | ENTPD8 | SLC9A4 | FGF17 |
| CTD-3080P12.3 | DHDH | PCDHA12 | KANSL1L-AS1 | DNAJB3 | SSTR2 |
| LINC01020 | LOC105378098 | SAMD11 | ATP11AUN | CYS1 | SLC5A12 |
| NDUFA11 | LOC729681 | C4orf19 | FREM2 | DCAF4L1 | LHFPL3-AS2 |
| TMEM86A | LOC101929420 | FER1L6-AS2 | ALDOC | C1QL3 | TNFRSF17 |
| LINC02198 | HOXC8 | FUT6 | SLC17A1 | SLC6A19 | SLC13A1 |
| GTF2H2B | LHX1 | SLC22A7 | FUT3 | DENND6B | LOC407835 |
| PRKAB1 | MYF6 | LOC105375800 | SIM1 | FNDC9 | LOC101928782 |
| NROB2 | ACKR2 | MIR429 | SAFB2 | LOC101927972 | PAPPA |
| CKMT2-AS1 | HNF1A-AS1 | GLIS3-AS1 | HOGA1 | RHPN1 | LINC01139 |
| C16orf96 | PLEKHA6 | LOC105379807 | LINC01942 | RNF208 | RNF213-AS1 |
| KCNK5 | GCGR | FAM27E2 | AACSP1 | LINC02530 | HOXC6 |
| DCDC2 | PRDM7 | GAS2L1P2 | CLEC18B | LINC01108 | TCAP |
| ASCL3 | C11orf86 | LRRC37A5P | UGT3A1 | MRPL36 | COL4A4 |
| ELOA3D | ADIG | TFCP2L1 | ASS1 | TBC1D3L | IQCF6 |
| MFAP3L | LOC101927391 | TAS1R3 | SOWAHA | LOC730338 | CELSR1 |
| CCDC153 | PCDHA13 | STPG3-AS1 | TMEM89 | TPT1-AS1 | |

Liver

| | | | | | |
|---|---|---|---|---|---|
| LOC729737 | ABCC6 | ZNF618 | ACVR1 | TOP1P1 | CXADR |
| ALDH1L1 | CAMSAP3 | SMIM32 | AKR7A2P1 | STXBP2 | GLYAT |
| LINC01770 | NOP9 | TRIB3 | GCAT | SCP2 | NOG |
| TRIM26 | RCE1 | ECHDC2 | INS | ALG11 | ADH1C |
| SPSB3 | TBC1D2 | EIF2D | CWH43 | CP | AKR1C6P |
| MASP2 | UQCRH | LOC100507564 | PFN1P2 | DYNC2LI1 | AKR1C8P |
| HYAL3 | GATA4 | CD163 | MROH2A | LINC01741 | LBP |
| CES3 | CCL16 | XAF1 | LINC01719 | PRKAG2 | ARRB2 |
| LINC01134 | ABCA6 | FMO3 | NBPF13P | MEIOB | HAO2 |
| PNPLA2 | ESPN | ACSL1 | BCO2 | PLG | SPP2 |
| HDAC10 | RNASE4 | STK31 | PXMP2 | LINC00862 | PALM3 |
| AFP | HSD17B7 | HMGCS2 | TRAF7 | LOC150051 | LPCAT3 |
| NEURL2 | NIT2 | CYP1A1 | FGFR3 | HMGCS1 | CMBL |
| EML3 | NSUN2 | NEK4 | B3GALT6 | SLC7A10 | BMP2 |
| RAN | RBIS | PRG4 | ZFP1 | SNAPC4 | KCNT2 |
| PSD4 | MYO1B | C1S | ACVR1C | SORBS2 | VAV2 |
| CHN2-AS1 | OCLN | SUV39H1 | AGPAT2 | MASP1 | MYORG |
| TRPV1 | PROZ | JMJD4 | AKR1C2 | PANK1 | TM7SF2 |
| NPPA-AS1 | SHD | MUC20 | EFNA2 | ACO1 | FAM99A |
| MSRA | CLCN5 | SERPINA12 | RASSF7 | PEPD | FAM99B |
| GLB1L | TCEA2 | SLC29A2 | NPY4R | ERLNC1 | ASB13 |
| FGL1 | ZFAND2A | TTLL2 | PMPCA | RMDN3 | SYT7 |
| TSSC4 | HNF1A | SLC25A10 | NECAB2 | TGDS | NGEF |
| C1RL | ANKS4B | NR112 | RASGEF1B | FAM20A | SERPINA1 |
| AIF1 | ACSF2 | MVK | DCST1 | CYP2A7 | PAH |
| KLHDC7A | VPS37A | ABCB4 | GJB2 | KHK | PNMA6A |
| CROCCP2 | SMAP2 | LOC389831 | MDH2 | LOC91548 | ZFAND4 |
| MST1P2 | RANBP1 | RUSC1-AS1 | UGT2B10 | LPGAT1-AS1 | HDC |
| ESPNP | SCN9A | GEMIN8P4 | ACMSD | SLC26A1 | HP |
| LOC102724562 | MRTO4 | DUX4 | NUP88 | NR113 | CYP4F3 |
| ARHGEF10L | C15orf62 | LOC101928118 | HABP2 | PROX1-AS1 | RAPSN |
| TNFSF14 | UGT2B4 | HNF4G | WDR54 | RNU6ATAC35P | GPS1 |
| AKR7L | TMEM53 | VEGFA | CBY3 | LINC02257 | SLC25A22 |
| PON3 | FN1 | ABCG5 | COL18A1 | SFTPC | CYP4A22 |
| GPANK1 | CNPY3 | SSTR1 | DENND1C | TBCEL | PUS3 |
| DPYS | CFHR4 | SRD5A1 | SLC17A3 | SORD | USF2 |
| GSDMB | C2 | ERRFI1 | CFHR5 | NSUN6 | SGMS1-AS1 |
| SLC19A1 | ASGR2 | C12orf43 | CYP21A2 | HNRNPA1 | TMEM63B |
| ZNF436-AS1 | IDNK | ELP2 | DAB1 | CFH | SIGMAR1 |
| KNG1 | TSLP | LDAH | APOL1 | LINC01736 | CCL3 |
| AKAP1 | MRS2 | RASSF10 | HPR | SLC6A1 | PRLR |
| PCYT2 | GC | EPHB1 | IL1RN | SLC22A9 | SLC2A10 |
| NR1H4 | ALDH8A1 | OGDHL | IL6R | DGAT2 | CDO1 |
| SNAP29 | CFI | PAX6-AS1 | FGFR4 | CDKN1C | TUBE1 |
| RAC3 | ARL14 | SNHG1 | MRGPRF-AS1 | MYT1 | MIGA2 |
| DMRTA1 | AGTR1 | GRB10 | POP4 | GHR | NUDCD2 |
| ST6GAL1 | MAP3K13 | PGLYRP2 | LINC02584 | EI24 | EME2 |
| CLRN3 | SLC39A14 | NBPF20 | SHANK2-AS3 | CCL18 | CBLN4 |
| GREM2 | DGCR5 | SNAPC5 | LRCOL1 | SOX4 | BLZF1 |
| CBSL | SERPINA4 | ERBB3 | EIF4E2 | LOC105369747 | SLC27A2 |
| ZNF503-AS2 | LOC399815 | SORL1 | PIGH | STIMATE | IGFBP1 |
| ADSS1 | CPS1 | ENPP1 | LOC283214 | C8B | RMDN1 |
| HLX | ERMAP | ADGRG6 | AQP9 | CPB2 | ALKBH2 |
| LINC02679 | NELFE | CPLX1 | FAM135B | CHRNA4 | SLC2A9 |
| RAB4A | SPRNP1 | UNC5CL | ACSM5 | H6PD | C9orf116 |
| MBL1P | TMEM106A | RIPK4 | PCSK6 | TMEM41A | PHGDH |
| LINC01519 | NIPAL1 | HCN3 | RORC | UNC93A | C7orf33 |
| CYP3A4 | CDPF1 | GLYATL1 | MGMT | TLR4 | NCMAP-DT |
| SGK2 | LOC143666 | LOC283194 | ESR1 | LLGL2 | PPP1R3B |
| PEX11A | NR5A2 | SCD | BCHE | MIR1180 | PSTK |
| HECTD2-AS1 | LOC171391 | KIN | PON2 | SNX10 | LHX3 |
| TOLLIP | PROX1 | ARL6IP4 | TF | FGB | LRWD1 |
| ARHGEF40 | SFXN5 | OVGP1 | OIT3 | ZPR1 | GSTZ1 |
| MRPL55 | ARG1 | THAP11 | PGM1 | PLPPR4 | LOC102724720 |
| NEU4 | IGF2-AS | EDF1 | LINC02753 | CPT1B | FBXO17 |
| IL37 | CYP4V2 | PPFIBP2 | IFT46 | HPF1 | LINC00939 |
| AGT | IL31 | SELENBP1 | MUC3A | NBPF19 | LOC100128276 |
| THOP1 | ENTPD5 | DRD1 | ADM | TH | LY75 |
| EEF1D | HHIPL2 | RPS9 | FGFRL1 | OASL | F13B |
| NOP53 | ADRA1A | FXYD5 | UGP2 | DCST2 | GATM |
| QDPR | LRRC45 | PKLR | TMEM186 | A1CF | SLC30A10 |
| CDH2 | SPAG7 | MYL6 | ACADS | LRP1-AS | ANG |
| F10 | SHMT1 | NKX2-8 | MGST1 | GLYCTK | TOX3 |
| MFSD2A | GPR31 | RHBDL1 | LOC101929384 | KYNU | SERPINA3 |
| PRKAB2 | RREB1 | SSTR5 | SLC6A13 | G6PC | NUGGC |
| ITIH2 | MTHFD1 | RDH16 | DUSP10 | ETNPPL | AQP3 |
| TDGF1 | IGF2 | AFM | ZNHIT1 | PLPPR1 | SLC6A12 |
| HAGH | ZFAND6 | RNF43 | ZNF503 | IL27 | AKR1C3 |
| FNDC5 | C3orf22 | TFR2 | KDM8 | SLCO2B1 | STRAP |
| LINC02624 | FOXD3 | TMEM101 | HSCB | CYP2D7 | MTRNR2L8 |
| HS3ST3B1 | UGT2B7 | CLDN9 | C1RL-AS1 | ACAD9 | CELF5 |
| IL1RAP | NAV2-AS5 | TNFRSF25 | INSIG1 | ABAT | COPS7B |
| CDKN1A | LOC100126784 | F7 | SNX17 | ACACB | TSKU |
| GALNT18 | SARM1 | ATP2B2 | SLC7A9 | GDF7 | IRF6 |
| HSD3B2 | EHHADH | NADK2 | SLC38A4 | PID1 | TPTE2P5 |
| ITGB2 | HAL | PCK2 | MLIP | ARMC5 | SLC13A5 |
| CPB2-AS1 | HPN | AKR1C4 | GPX2 | CD1D | PLA2G12B |
| TLX1 | ATF4 | AVP | LRRC75B | SHH | IGSF8 |
| BAAT | NBPF14 | LIPC-AS1 | COQ6 | EPHX2 | SARDH |
| GFRA1 | INSM2 | GABRQ | MRPS26 | ARL2 | MREG |
| PPP1R1A | IHH | GRIN3A | SLC15A5 | LOC105371485 | CPPED1 |
| GOT1 | ATF7IP2 | CEND1 | SLC2A2 | DHCR7 | PEX6 |
| SEBOX | PHYH | ITIH1 | ABCC6P1 | TRO | LINC00671 |
| C10orf62 | TFG | MYCNOS | POLR1E | HRNR | C4BPA |
| RNF123 | LOC100506358 | SLURP1 | IQGAP2 | RND1 | CTXND1 |
| ENPP7 | MLYCD | CKAP2L | PROSER2 | SULT1B1 | NBR2 |
| ZNF497 | ABCC3 | GLT1D1 | SLC7A5P2 | ALOX12P2 | MIR2117HG |
| MYO18A | PCNA | ARMC6 | PRR3 | ALDH4A1 | ARSL |
| LINC00449 | DGCR6L | TYRO3P | EPHA1 | MTARC2 | PCAT6 |
| MARK4 | TMPO | TMEM114 | FTCD | CLSTN3 | TBC1D14 |
| FGF14-IT1 | SERPINA5 | MICOS13 | CPN2 | ATP6VOD2 | RANBP10 |
| RAD54L2 | GCKR | HNF4A | SMG1P6 | LRP1 | CYP2B6 |
| APOE | PSAT1 | FBXL6 | DIRAS2 | ZSCAN22 | IDH1 |
| UROC1 | CROT | HNMT | TAF1A | OSGIN1 | NKX2-1 |
| NPC1L1 | MAD2L2 | POGLUT2 | SUCLG2 | C6 | TGM3 |
| LINC00399 | CABP2 | ACTL6B | ABCA8 | NUBP2 | NRBP2 |
| CDHR5 | SERPINA13P | RNF151 | CES1P1 | HAPLN4 | GLYATL3 |
| LINC01070 | CPNE6 | KLC4 | DKFZP434H168 | CNTFR | CAPN5 |
| AKR1D1 | LINC00677 | SS18L1 | VKORC1L1 | F9 | F11 |
| F10-AS1 | H3-4 | KLHL25 | ABLIM3 | TSHR | LINC01482 |
| GRTP1-AS1 | SLC17A9 | HSP90B2P | NKX2-4 | MAGOH2P | CASC17 |
| LRRN4 | REREP3 | DCAF11 | NLRP6 | TMEM220-AS1 | MT1G |
| ABCC9 | BOD1L2 | LOC100507472 | C5 | CD2BP2 | ZCCHC9 |
| LOC254028 | RAPH1 | PCSK6-AS1 | PPDPF | LRP5L | SLC66A2 |
| GPR182 | ADGRG7 | IGFALS | CPN1 | PARD6A | SNTB1 |
| PFKFB1 | PRR15L | REN | SMG1P7 | ALG3 | MIR23B |
| APOH | SOX5 | SLC16A2 | CYP3A5 | S1PR1 | FAH |
| FGF21 | ZNF646 | AKR7A3 | VGF | ZNF444 | GLDC |
| FRG2 | RNASE13 | HCFC1R1 | A1BG | RIBC1 | BHMT |
| ADH1B | LCP1 | GOT2 | STEAP3 | ASGR1 | ACTG1 |
| PDIA4 | CSNK1A1P1 | FOXQ1 | GDA | AOX1 | ROCK1P1 |
| CYP2C19 | CYP4F2 | CAMK2N2 | HPX | COLEC10 | SULT2A1 |
| CYP2C8 | ABHD17B | ACOX2 | KCNJ3 | CYP1A2 | C3 |
| LRR1 | BAIAP2 | NID1 | CYP2C18 | MRNIP | GACAT2 |
| MLXIPL | SLC25A15 | WDR72 | LOC101928737 | PLET1 | LDLRAD4-AS1 |
| CREB3L3 | IFRD2 | CUX2 | CA5A | MOCOS | SAA1 |
| CHST13 | ETS2 | FGG | PRR18 | KDF1 | LY6E |
| SYT17 | SORD2P | FBP1 | NAXD | FBXO39 | DSG2-AS1 |
| CYP26A1 | ONECUT1 | F2 | EPS8L2 | SEMA4G | NUDT22 |
| LRP3 | EDAR | TMEM203 | C1QTNF12 | SLC22A25 | SLC12A7 |
| DEPDC7 | LOC102724908 | SLC1A1 | PRAMENP | LAMA5-AS1 | TTC6 |
| BHMT2 | CES1 | LRRC3 | SEMA6B | MYH14 | FTCD-AS1 |
| FOXA2 | GGH | POFUT1 | EXOC3L4 | PC | VNN1 |
| CYP7A1 | HPN-AS1 | ST3GAL5-AS1 | SYT12 | LOC100130587 | F5 |
| SNHG22 | OXT | THNSL2 | HSD17B2 | PHLDB3 | TEX30 |
| CARMIL3 | FAM20C | CYP4F11 | CPS1-IT1 | LINC00189 | PTGDR2 |
| ANO9 | PROS1 | LOC389602 | GBP7 | AMN | SLC6A1-AS1 |
| HSD17B6 | DDC | GMNC | DBP | INA | MAP2K1 |
| GBA3 | CYP2G1P | RPL10A | KCND3 | SEC14L3 | TMPRSS6 |
| MINCR | CYP2B7P | TTC38 | GPAM | PSMD4 | ACADSB |
| SIRPA | LRRC8E | SLC25A47 | PREB | WWP1 | DRG2 |
| LINC01924 | GYS2 | ACY3 | NHSL1 | LRRC3-DT | ZBED6CL |
| ALDH2 | TRPM8 | CCNYL1 | LOC100286922 | LINC02575 | LINC01988 |
| LINC00683 | MAZ | LPA | IGFBP6 | TSPEAR-AS1 | KCNK17 |
| LINC01002 | ACSM2A | PEX19 | HGFAC | LINC01424 | GSTA1 |
| FGF19 | CYP2C9 | STEAP3-AS1 | PRAP1 | LINC01547 | HES4 |
| EGFL7 | ACSM2B | LOC107105282 | CERS2 | MEGF9 | MIR8064 |
| ABCC11 | PIPOX | ASL | NUP210 | LOC284865 | AMDHD1 |
| VM01 | LMNTD2 | DLL4 | PDYN-AS1 | LINC00896 | MIR126 |
| JAML | TRIM15 | ZNF467 | SERPINE1 | ARID3C | HSF5 |
| VASN | FOXD2-AS1 | NOTUM | CISH | HAO1 | MAP2K7 |
| PRKAG3 | RAMAC | IGF2R | LINC01713 | C6orf47 | MOGAT2 |
| PRODH2 | COL6A2 | SLC45A2 | LINC01428 | FOXA3 | DAO |
| ASIC5 | PHF10 | ALDH1A1 | SEC14L4 | LOC100507599 | SLC31A1 |
| FM05 | SHPK | CYP39A1 | ADM2 | FGF3 | IFNLR1 |
| TTR | LGI1 | CFAP57 | FUT5 | NAGA | PECR |
| TTPA | CGREF1 | MTTP | KCNG2 | APBB1IP | ABCC2 |
| SPATC1 | EMG1 | RAPGEF4-AS1 | ETFB | CCS | LOC101927374 |
| C3P1 | ASCL1 | SERPINA10 | GDF15 | LINC02558 | TOMM70 |
| ADH6 | MSMO1 | LRCH4 | LOC284788 | CPM | KCNJ8 |
| A2M | MGAT4B | ADGRA3 | NPAS2 | C1orf210 | CLDN5 |
| ACP2 | LINC01320 | GAL3ST2 | APOL6 | PPL | SLC38A3 |
| KRTAP10-5 | LINC01913 | PBLD | PNPLA3 | POU3F1 | KMO |
| LIPG | CDHR2 | TTBK1 | RPL23A | HPD | PON1 |
| ABCG8 | LOC100132215 | GEN1 | RBM46 | TEDC2 | UGT1A1 |
| C11orf24 | CYP4A11 | XDH | LOC100287792 | CNGA1 | IDUA |
| SLC17A4 | SLC47A1 | PCK1 | HSPA1L | MROH2B | SPDYC |
| AMACR | CRYBB3 | HYLS1 | SLC12A5-AS1 | NDUFA6-DT | NLRP11 |
| OPN3 | XYLB | LKAAEAR1 | SC5D | ELOVL6 | SULT4A1 |
| PITPNM2 | FOXH1 | CCDC150 | LIPC | KCTD6 | ZNF324 |
| GPR88 | ALMS1P1 | NAT2 | ADCY1 | RNF130 | ELP1 |
| NUDT7 | ZIC1 | AOX3P-AOX2P | PIK3C2G | FAM149A | CRP |
| F12 | SERPINA9 | AGXT | TSPO2 | ALG1L | PMS2P1 |
| DENND2D | SULT1E1 | CYP27A1 | PPIF | MEP1B | KMT2E-AS1 |
| MOGAT3 | CST7 | PMCHL2 | LDC1P | ZNF367 | SLC22A10 |
| LOC100507389 | TBX10 | CD3D | GSTA7P | ASPG | MAL2 |
| HEPACAM | SKIV2L | LOC101929109 | LOC101927136 | HNF1A-AS1 | ASS1 |
| ADRB1 | BDH1 | NCRUPAR | SELE | ZBTB5 | TEKT1 |
| FAM83C | TAT | GMPPB | PFN3 | SRD5A2 | DMGDH |
| TMEM37 | TDRP | ORAI3 | LINC02535 | TTLL4 | LOC155060 |
| SLC17A2 | CYP4F22 | C9 | CS | SLC7A5 | RFNG |
| FAM106C | SESN2 | LINC00514 | PTP4A1 | PLEKHA6 | PROC |
| AGXT2 | TPPP2 | NSUN7 | SAP25 | EPO | LOC100506302 |
| CGN | THORLNC | ADH1A | LINC02532 | GCGR | LOC157273 |
| FFAR4 | HIRA | LINC01848 | LINC00222 | ANGPTL3 | PPP1R26 |
| KLHL6-AS1 | FMO4 | LOC101927078 | TPI1P3 | PRDM7 | CLRN2 |
| LINC01991 | APMAP | TAB3 | ACOT12 | RPS28 | HAAO |
| KLHDC10 | KLKB1 | KCNK5 | ZBTB39 | C11orf86 | RCN3 |
| MIR570HG | F2RL2 | MTNR1B | RTL9 | MLANA | APEH |
| B4GALT1 | TSPEAR | MIR3936HG | LOC101928140 | CAT | ALDH1B1 |
| LINC02012 | SHROOM1 | FBXL21P | TAAR3P | FASN | DHODH |
| ALDOB | GK3P | LOC102724265 | ABCA1 | ELFN1-AS1 | AKR1C1 |
| ALAD | CHST5 | ABCB1 | FAM221B | FETUB | CTSO |
| FAM157A | LRRC42 | SALL1 | SMLR1 | LOC101927391 | FGF4 |
| NOS1AP | TTC9B | MAT1A | HSD17B13 | AZGP1 | REEP6 |
| H3C6 | LINC02363 | MMUT | CYP2A13 | PDE6C | HGD |
| SDS | LINC02362 | TNFRSF12A | CYP2A6 | LOC105375304 | C4orf19 |
| ECHDC3 | POR | XAB2 | ABHD14A | GJB3 | LTC4S |
| CHAD | SLED1 | IMP3 | SERPING1 | DKFZP58611420 | PIK3AP1 |
| GNAT1 | PLIN4 | ONECUT2 | LPAL2 | OXER1 | MAL2-AS1 |
| FH | DBET | TM6SF2 | LINC01558 | RHPN2 | LOC101927543 |
| USP30 | VNN3 | ITIH4 | LINC00574 | GNPNAT1 | LINC01151 |
| GNAO1 | CTD-3080P12.3 | GATA6 | PTGR1 | CCT6P3 | FAM83A-AS1 |
| LOC344967 | ITIH3 | EFCAB12 | ZNF408 | INAFM1 | PROCR |
| LINC00682 | LINC01018 | UGT3A1 | INHBA | PZP | FUT6 |
| CCL19 | GPR61 | SLCO1B1 | MYF6 | PMS2P5 | CPOX |
| DCUN1D4 | HRG | ELOVL2-AS1 | DYNLL1 | OAF | PKD2L1 |
| DECR2 | TENT4A | CFHR1 | ABCB11 | DTX2P1- | DHTKD1 |
| | | | | UPK3BP1- | |
| | | | | PMS2P11 | |
| HCN1 | CYP4F12 | MAFA | NEUROD6 | PMS2P9 | CDC42P3 |
| ELF3 | LOC257396 | CACNA1H | APOB | FBXW5 | MIR4664 |
| LOC105377267 | ANO1 | C2-AS1 | APOBR | SOCS6 | C1orf220 |
| DNASE1L3 | ECHS1 | CYP21A1P | ACKR2 | LECT2 | C8A |
| LBX2 | DHCR24 | SERPINA6 | PAPSS2 | AZGP1P1 | ISOC1 |
| OPLAH | UPB1 | NPBWR1 | PCSK9 | LOC105375423 | SLC7A2 |
| CHDH | LOC101928858 | ST8SIA3 | NAMPT | SLC25A20 | TPH2 |
| TECTB | LOC101929372 | RTN4R | IGSF23 | SLC22A1 | CEACAM16 |
| LINC01239 | MST1L | NAGS | LAG3 | SMPD5 | MMP15 |
| TBX15 | LNP1 | AMIGO3 | NR2F6 | CLPTM1L | KRTAP5-7 |
| CYP2E1 | SMOC1 | ENTPD8 | LOC100129034 | ZFPM1 | C2CD4B |
| LOC101928195 | SLC19A3 | PLIN5 | TAS1R3 | LINC00659 | DEFB132 |
| MTMR4 | LOC644090 | LEFTY1 | DBH-AS1 | CALML3 | TUBA4B |
| AQP7P1 | TMC3 | MST1 | LINC02247 | ITPR2 | SP5 |
| CLDN6 | FAM83G | LINC00261 | OMP | APOA4 | TBC1D3B |
| PCAT7 | ANKRD34A | KANSL1L-AS1 | PRSS53 | GPC6 | SPATA46 |
| BCL3 | ESD | LINC00319 | GCH1 | GTSF1L | DNAH8-AS1 |
| PEX16 | SERPINA11 | SPTBN2 | DNAJB3 | FEN1 | ENHO |
| ERG28 | TDO2 | SOX15 | SPRYD4 | RNASE8 | MIR12136 |
| LRRC37A5P | SDC4 | LINGO4 | RGPD1 | SLCO1B7 | SIGLEC15 |
| DNAJC25 | PPM1N | SGF29 | PCSEAT | RTL8B | MIR10398 |
| CCL22 | CUTC | LOC105371566 | PAQR9 | EMX1 | ELFN1 |
| SMCO4 | CCDC158 | SLC17A1 | TTC31 | ISM1 | ERAS |
| GSTA2 | KRTAP10-4 | FAM83H | MTRNR2L2 | UQCRQ | BHLHE22 |
| SOCS1 | KRTAP10-6 | | | | |

Lung

| | | | | | |
|---|---|---|---|---|---|
| RHOJ | LDB2 | NEDD9 | CCR5 | ZEB1 | CTNNAL1 |
| TFPI | TLR8 | OCLN | RBM45 | VIPR1 | NPNT |
| LINC02861 | CALCRL | EFNB2 | CLDN18 | NOX3 | CLCA3P |
| GBP1P1 | PRL | IL5RA | GJA5 | GOLGA6L22 | DNM3OS |
| FENDRR | LMCD1 | FAM177B | LINC01606 | GBP1 | VSIG2 |
| DACH1 | HLA-DQA2 | RAPGEF5 | CCM2L | ARHGAP29 | CASS4 |
| TRIML1 | DISP1 | LOC101929538 | LINC00987 | ERG | MROCKI |
| ARHGEF15 | TDRD10 | C3orf36 | ICAM1 | ICAM5 | BTNL8 |
| SYT17 | CXCL8 | HACD4 | CCDC102B | SMAD6 | LOC101928988 |
| EWSAT1 | LOC101929586 | IL2RA | PAPOLB | CDKN2B | TEK |
| IFNGR1 | LOC101927727 | RALA | VIM | SLC34A2 | CARD6 |
| KCNJ15 | LINC02735 | KCNS3 | LINC01478 | CYP4B1 | LINC01929 |
| ADGRF5 | EMP2 | PTPRB | A2M | ABHD17C | TENM1 |
| KHDRBS2 | RPL23AP32 | DLC1 | SH2D1A | ADGRF5-AS1 | SLFN5 |
| RASIP1 | CFLAR-AS1 | S1PR4 | PRNT | IL7R | C5AR1 |
| LINC01430 | LINC00589 | CYYR1-AS1 | NDNF | MGP | TM4SF18 |
| LINC00690 | TNIP3 | CRYBA4 | STX11 | LINC02475 | RAB32 |
| TACSTD2 | LINC02516 | BTN3A3 | HMCN1 | LINC02160 | KDR |
| SPRY4-AS1 | LOC100506885 | CLDN20 | SLCO2A1 | AHR | HAS2-AS1 |
| SHROOM4 | PDK3 | AFDN-DT | SLFN12 | HEG1 | IDO1 |
| LOC401312 | GIMAP1 | SLC6A4 | NPC2 | DOCK4-AS1 | PLVAP |
| LOC101929415 | KANK3 | LOC101927513 | OTULINL | LOC102723534 | LRRC55 |
| NBAT1 | LRRC70 | | | | |

Lymph Nodes

| | | | | | |
|---|---|---|---|---|---|
| LINC01342 | NFKBIL1 | KLHDC9 | SIT1 | CD74 | LOC105378586 |
| UBAC2 | LOC100996583 | PPAN | FCGR3A | NRM | C11orf21 |
| LINC02606 | PIK3CD-AS1 | PIK3CD-AS2 | TAP2 | HLA-C | LOC105376805 |
| SHOX2 | TNFSF14 | CD53 | ZFP36L1 | IFI30 | LSM4 |
| FTL | PLEKHO1 | ARHGAP9 | CYSLTR1 | LOC101928163 | TAP1 |
| COA1 | LINC02574 | PIK3CD | MS4A1 | PTGDR | C12orf57 |
| TLR7 | AGAP2 | IGLL5 | THEMIS2 | BTN3A2 | MSL3 |
| IL21R | LSP1 | SLAMF1 | SH2D3C | LOC100507634 | PAX5 |
| IF144L | LINC00324 | MBNL1-AS1 | STRADA | MIR339 | CD69 |
| LINC01160 | MPZ | VAMP1 | PSMB8-AS1 | NBPF25P | PIK3CG |
| ABI3 | PATL2 | FBLN7 | AMZ2 | HLA-DRB1 | HLA-DPA1 |
| TEAD2 | CD1A | PLEK | DOK2 | FCRL3 | CD48 |
| FCRL2 | RPS27A | PSMB8 | CD3E | P2RY10 | ARHGEF6 |
| ICAM3 | VOPP1 | LINC01353 | ILDR1 | ZNF10 | SP140 |
| HLA-E | TAGAP | LILRB1 | IL10RA | LINC00582 | CENPM |
| LINC02649 | SEPTIN6 | KCNA3 | ACD | FAM238A | ADIPOQ |
| SVIL2P | IL2RG | LOC101929431 | TBXT | AIM2 | DLL3 |
| CLEC2D | TRAF1 | LILRA1 | SAMD7 | ST6GAL1 | P2RX5 |
| MCL1 | PYHIN1 | CXCR4 | LAIR1 | ENTPD1-AS1 | LST1 |
| PRR29 | TYROBP | PDCD4-AS1 | CCR7 | BTN3A1 | BIRC3 |
| TREML2 | FAM53B-AS1 | NDUFC1 | AS-PTPRE | HLA-DOA | TOLLIP-AS1 |
| ORMDL3 | SUPT4H1 | GVINP1 | OXLD1 | PRELID1 | TRABD2A |
| ACTL9 | ITGB2-AS1 | DNAJC4 | TSC22D3 | BTNL9 | MIAT |
| NLRC5 | MS4A14 | IKZF3 | SOX12 | PIM2 | EVI2B |
| MGAT2 | HLA-DOB | IKZF1 | PTPRC | AVPR2 | C16orf54 |
| MUC4 | CHST2 | MMP9 | AANAT | CD1E | SPIB |
| CD27-AS1 | TBCC | MIR200CHG | FOXP3 | LOC374443 | IL24 |
| MTIF3 | C1QC | XPNPEP2 | ZPBP2 | ETS1 | ABCD2 |
| HLA-F | CETP | ADAM28 | LINC02422 | NOSIP | MEOX2 |
| IRF4 | HLA-DMA | BCDIN3D-AS1 | CABP4 | PNRC1 | HLA-DMB |
| CXCR5 | CLEC17A | SELL | LOC101928617 | SLAMF6 | GRK6 |
| CD247 | IL10 | PSMB9 | LINC02361 | PYCARD | HSPA1A |
| AKNA | LINC00563 | TCTN1 | STK24-AS1 | DOCK9-DT | UBAC2-AS1 |
| GFY | TNFAIP8 | BTN2A2 | GIMAP8 | BCL11B | ATF5 |
| RLN3 | ARHGAP5-AS1 | LILRB4 | RFTN1 | ATF4 | NRGN |
| CREB3 | DMRT3 | ACAP1 | SPNS1 | ARHGAP45 | OTUD5 |
| SELENOH | LILRB2 | LINC02280 | LINC00221 | NCR3 | FBXL15 |
| STK17A | TMEM35B | FAM71B | TRAF3IP3 | LOC105370792 | FCGR1B |
| LILRA4 | LOC100419583 | HLA-B | MYEOV | LRRC23 | HLA-DRA |
| JAK3 | HMX3 | LTA | TXNIP | LINC02205 | ZCCHC3 |
| CCDC155 | TUBA4A | TCL1A | IRF2BPL | WAKMAR2 | RTL8A |
| PRSS30P | HCFC1R1 | MMP25-AS1 | ARMH1 | ISG20 | IL21R-AS1 |
| BTN2A1 | RUNX3 | LOC606724 | MIR762HG | RIMBP3C | QRSL1 |
| CXorf65 | RHOH | HVCN1 | ZMYND10 | LOC100505942 | FNDC11 |
| HCST | CD1D | LCN8 | LOC100130950 | SCIMP | LIM2 |
| TMC6 | LOC284191 | LINC02090 | RFX5 | CD79A | TNFRSF1B |
| S1PR1 | PRKAR1B-AS1 | CD3G | TNFRSF13B | HLA-DPB1 | GPX7 |
| GADD45GIP1 | ALDH16A1 | TNFSF11 | RCSD1 | LOC101927166 | FITM1 |
| LAPTM5 | ZNHIT2 | VSIR | MGC16275 | DUS2 | HEXIM1 |
| BTLA | LINC01973 | GPR18 | BLK | GSTP1 | LY6E |
| MYL12A | TRIM22 | GPR174 | CD5L | WNT10A | MRPL40 |
| CCL5 | UBC | ZNF853 | ZNF426-DT | ADAMTS4 | STK17B |
| FAM81A | GPR65 | LY9 | CD209 | LOC105372383 | FCER2 |
| GADD45B | SLC6A16 | FCRL1 | HTRA2 | PSMB10 | CD180 |
| PTPN6 | NAPSB | RGS19 | CYBA | ANTKMT | FCHO1 |
| TNFAIP8L2 | FAM30A | MIR497 | TMEM107 | MOB3A | CCR6 |
| CASP10 | MIR589 | SYNGR2 | NDC80 | CD1C | SLFN5 |
| MPZL3 | RPS6KB2 | SIRPG | TIMM29 | ANKRD44-IT1 | LOC114841040 |
| LYSMD1 | LFNG | CYTH4 | LINC01891 | SLFN12L | SIRPG-AS1 |
| CLEC4G | IL7R | RALY-AS1 | TLDC2 | JSRP1 | FCMR |
| MIR1914 | ITGAL | RUNX1-IT1 | LY6G5C | LINC01547 | WNT1 |
| FCRL5 | APBB1IP | PVRIG | LOC101927051 | POU2F2 | MVD |
| LOC101929054 | SUCLG2-AS1 | FCER1G | LINC01215 | C1orf56 | BST2 |
| CD37 | CD79B | TMEM14EP | LOC100128164 | KRT9 | CASTOR3 |
| FCRL4 | LINC01097 | STIM2-AS1 | H1-1 | IL17C | TNF |
| ZNF575 | ATP5IF1 | MRGPRX4 | FLJ38576 | SSTR3 | BTN3A3 |
| MRPL41 | CD72 | ARRDC3-AS1 | CD300A | RGMB-AS1 | PCED1B-AS1 |
| LOC101929719 | TNFRSF12A | LOC285626 | PRR7-AS1 | LINC02222 | CTC-338M12.4 |
| LOC285766 | LOC101927730 | LINC01011 | LOC100506207 | CNFN | LOC108783645 |
| LOC285819 | LOC100270746 | LOC285847 | LOC401261 | TALAM1 | C12orf77 |
| TLR9 | GABARAP | NFKBIE | SASH3 | FOXF1 | NMB |
| KRTAP16-1 | RAC2 | LOC645177 | DTX1 | FASLG | CARNS1 |
| GPR183 | ZNF80 | UCP2 | UBALD2 | RNF44 | TIMD4 |
| LOC442497 | GZMH | FAM167B | B2M | TNFRSF8 | SERPINB9 |
| CLLU1-AS1 | GIMAP1 | CCAT1 | ST7-AS1 | ATP6VOE2-AS1 | LINC00996 |
| CYTIP | GALR2 | UTP3 | LOC389641 | PELI1 | NDUFB10 |
| TIGIT | GPR132 | LINC00861 | B9D2 | DEF6 | LOC103908605 |
| LOC100133920 | LOC100128593 | LPAR4 | LINC01560 | EXOC3-AS1 | LOC100996643 |
| BLOC1S4 | ANKRD34A | HSPA1B | PPM1N | PECAM1 | C17orf97 |
| RHEX | TMEM187 | P2RY8 | KLHDC7B | LOC101927322 | PLPP6 |
| ITK | TSPYL1 | | | | |

Ovary

| | | | | | |
|---|---|---|---|---|---|
| FBN1 | MXRA8 | RAD9B | MMP23A | KCNIP3 | MOSPD1 |
| TRPC4 | MIR4251 | PBX3 | PNMA6E | TSHZ3 | AFP |
| MDFI | FAM122C | CEP83 | LOC100128108 | MIR34AHG | ANTXR1 |
| MTOR-AS1 | NAV3 | BX | STK3 | TMEM161A | SLC25A34-AS1 |
| NECAP2 | LINC01783 | CSRP2 | RPS14P3 | PARD3B | KLHL38 |
| CASP12 | CPZ | SENP1 | MAPK8 | MMP23B | TNXB |
| RGS22 | GATA4 | TSBP1 | SMOC2 | COL21A1 | C21orf62 |
| ALDH1A2 | FAM163A | TMEM159 | SPSB1 | FBXO32 | MND1 |
| IGFBP3 | NRP1 | EGFLAM | ATP1B2 | ZMYM1 | MAGEB3 |
| PARVB | TPM1 | PITPNM3 | TAS2R30 | IQCD | ANLN |
| GLI2 | PPARG | PRTFDC1 | STRA6 | IGF1 | MSI1 |
| GDAP1L1 | CCDC185 | CELF2 | SCGB1A1 | BMPR1B | TTC39A-AS1 |
| PDGFRL | WNT5A | NUDT2 | LINC02861 | LAMB3 | DENND1B |
| TRPC1 | TXLNG | LINC01135 | TYRO3 | BICC1 | ZFP91 |
| DPF3 | THOC7 | RHBG | CALCRL | SGCE | C1S |
| PPP4R4 | LINC01788 | LRRC53 | CPA3 | ANKDD1A | RALGPS2 |
| ROBO2 | LINC01712 | SEC31A | UBB | CDK1 | ARMCX5- |
| | | | | | GPRASP2 |
| COL6A3 | CENPP | NR5A1 | RGS5 | LOC101928370 | KIRREL3 |
| ASH2L | SYT1 | DACT1 | GSTM4 | APLN | ORM1 |
| SIGLEC11 | PCMTD1 | DUSP26 | PPIL1 | OLFML3 | C17orf78 |
| MYMX | NGF-AS1 | LOC101928977 | SLC25A25 | LAMA4-AS1 | ADAMTS9-AS2 |
| CHURC1 | EBF1 | SEL1L2 | PSMD12 | STAR | TMEM120A |
| PARP11 | CHST15 | FBXL13 | LOC101928009 | CRB1 | SMIM1 |
| SULF2 | PPP1R18 | CTNS | SLAMF8 | SPDYE8 | SSC5D |
| USP44 | TMSB15B | IL17B | UFM1 | FER | H19 |
| VIPR2 | LOC101928372 | CCDC34 | BOC | HPR | NCS1 |
| LINC00626 | ARHGAP42 | PVT1 | LINC01142 | PLIN3 | BNC2 |
| ZNF589 | LAMC1-AS1 | KIF16B | ACTA2-AS1 | KLF4 | CD22 |
| HSPB3 | AIG1 | MELK | MN1 | RWDD4 | VEGFC |
| GREB1 | SIGLEC16 | ITGBL1 | SYNPO | CEP120 | AK1 |
| ERLNC1 | LEMD1-AS1 | TNFAIP3 | FAM171B | RBMS3 | CHL1 |
| PABPC4L | CARD11 | ITPKB-IT1 | RAB29 | DNMT3A | SPECC1 |
| DISC1-IT1 | G6PD | CYP2W1 | CHRM3-AS2 | PCDHGC3 | TK1 |
| PSD3 | MEIS3 | FAM122B | SHBG | SNCAIP | ELK1 |
| LINC02657 | KRTAP2-1 | SFTA1P | LOC101928453 | FXN | SAXO2 |
| KCNT2 | VIM-AS1 | SLAMF7 | LOC100130992 | VSIG10L2 | LINC00963 |
| CCL26 | FAM99A | LINC00837 | SYNPO2 | TRIB2 | AMHR2 |
| THEMIS | LINC01605 | ZNF32-AS3 | LINC02658 | PNMA6A | MRAP2 |
| FAS | TSHZ1 | BABAM2 | MIR744 | ARHGAP22 | FAM170B-AS1 |
| SPART | LTBP4 | PCSK5 | TSHZ2 | FAM133CP | SLC16A9 |
| ITM2A | ALLC | LOC101928961 | LRRTM3 | TMEM200A | ADAMTS16 |
| ZFP92 | SERPINE2 | TMEM138 | CCDC170 | TMEM174 | NEIL2 |
| LINC00974 | LOC101929165 | LRMDA | DCN | FPGT | CCKAR |
| PLCE1-AS1 | PLSCR4 | RRAD | RBP2 | GLIPR1L2 | AQP4 |
| SAMMSON | PCBD2 | PEG3 | DNMBP-AS1 | CCL24 | ENDOU |
| NFIX | IFI27 | SNORA87 | LINC02626 | QPRT | SFXN4 |
| ROBO4 | LOC283038 | ASB7 | SLC27A6 | TCERG1L-AS1 | AEBP1 |
| APOBEC3A | MIR202HG | CAMK1D | GNG3 | FXYD2 | LRRN4CL |
| LINC01150 | MRPL23-AS1 | SYTL4 | POLA1 | TUB-AS1 | HMGA2 |
| CAND1.11 | NOX4 | FGF5 | RIOX1 | GAL3ST4 | SLC16A14 |
| PRRT1 | BBOX1-AS1 | GNAQ | TRABD2A | PTPN7 | LOC100507384 |
| TCEANC2 | CSF3 | GNG11 | LIME1 | MAPKAP1 | TCTEX1D1 |
| PI15 | BRD7 | OOSP1 | ZC3HAV1L | BRINP2 | SLC6A6 |
| IL34 | RCL1 | OSR2 | NUSAP1 | LIPN | GPR1 |
| C4orf47 | TEX26 | VPS13B | IQUB | FAM86C2P | ACSL4 |
| STK32A | FGFR4 | CDKAL1 | C3orf70 | KCND2 | ATP1B3 |
| DENND2A | LOC646029 | OR2F2 | MIR4300HG | CCDC141 | NAT8L |
| WT1 | TTYH2 | CRYAB | CLIC2 | ESR1 | MIR629 |
| FAM181B | UBTD2 | ENC1 | ZNF431 | THBS4 | C1R |
| ACTR3B | RNF214 | RGS7BP | RPS6KA6 | KIAA1522 | INMT |
| MAG | RPP40 | LSM7 | LINC02098 | FHL2 | NTM-AS1 |
| SNAI1 | GPR157 | TSPAN5 | ACTA2 | SPOCK2 | PRMT8 |
| C5orf58 | LINC00336 | BICD1 | SOCS5 | FAM86FP | ANGPTL5 |
| S100B | PDGFRA | ANKRD66 | LINC01489 | RERG-AS1 | ARHGEF28 |
| AOC3 | SOX5-AS1 | MVP | RAB9A | TTC8 | HEYL |
| KERA | DENND5B | CLIC6 | RYK | LINC02450 | PTGIS |
| PHYHIP | P2RX2 | TPM4 | ADAMDEC1 | SLC25A37 | LINC02395 |
| RSAD2 | NXNL1 | IGFBP2 | MIR1180 | ACOT8 | AMIGO2 |
| TESPA1 | LOC100240735 | IRF8 | SLC8A1 | LRP1-AS | PSMA8 |
| LINC02388 | LINC02454 | LINC02762 | LINC02420 | CEP295NL | NT5C3A |
| MFAP2 | CDON | JPH4 | LINC02392 | LINC02412 | LOC105369911 |
| SHISAL2A | PID1 | CORO2B | TRDMT1 | DAPP1 | LINC00485 |
| FUNDC1 | MAOB | CASC18 | ARMC4 | DHRS2 | AP1S3 |
| GNA15 | FKBP5 | WNT2B | NRAV | NAT8 | ZEB2 |
| CLIP1-AS1 | NAAA | LOC646588 | LINC02370 | NTHL1 | ASPM |
| LINC00424 | LINC00540 | SACS-AS1 | TPTE2P1 | MTUS2-AS1 | LINC00297 |
| PHETA1 | LINC01048 | LEMD1 | TGFB111 | LINC00330 | ZNF70 |
| SCT | IFI35 | GPR19 | CTIF | MCC | INTS6-AS1 |
| CDC7 | ZNF275 | CENPI | CD6 | CFP | TSKS |
| ST6GALNAC3 | THBS1 | EHD4 | TFF3 | COL1A2 | CCN1 |
| NANOG | GALNT15 | TOB1 | ATF5 | CIDEC | G2E3-AS1 |
| VCAM1 | ADH1B | CCDC42 | ANO4 | LINC00519 | RESF1 |
| LOC102723604 | RAB9B | SYNGR1 | KRTDAP | TMEM215 | NSD3 |
| OSTC | USP51 | JPT2 | GRIK1 | NCBP2L | WWTR1 |
| DACT3 | GNG2 | LOC101928504 | LINC02328 | DPYSL3 | DNAAF1 |
| TCL6 | PLS3 | CTAGE1 | MEG9 | LINC00524 | LINC02314 |
| DIO3OS | LINC00677 | CD55 | GPALPP1 | LOC646652 | RNASE13 |
| LIF | KNDC1 | FBXL7 | TEX52 | YARS1 | KRT23 |
| PCARE | TNFAIP8L3 | MYLK2 | KLHDC8A | RORA-AS2 | MGC15885 |
| JAK3 | TXNIP | LINC02204 | THSD4-AS2 | AREL1 | WWOX |
| OGN | SAV1 | PPIAP46 | ZCCHC18 | TRMT44 | SRPX2 |
| HNF4A | PNLIPRP2 | GOLGA2P10 | EFEMP1 | LINC00933 | HNMT |
| MRAP | EMX2 | GABARAPL3 | CRTC3-AS1 | RAB23 | LINC02207 |
| LINC01580 | NPR3 | LINC00924 | NR2F2-AS1 | PGPEP1L | LOC400464 |
| MAMLD1 | LINC02348 | SERHL2 | NUDT1 | GBP5 | NOXRED1 |
| FAM185A | LINC00689 | ZDHHC19 | NPIPA5 | POPDC2 | GLIPR1L1 |
| EPS8L3 | NFE4 | CRYM-AS1 | KIAA1191 | AP4B1 | SSPN |
| RALBP1 | VAMP8 | KRT37 | RBM38 | HAGLR | SPIN1 |
| LOC105371267 | CD244 | LINC02126 | PTPN21 | S100A4 | PWWP3B |
| SINHCAF | EMCN | SGCD | PDCD4 | WT1-AS | LOC100129617 |
| P4HA1 | LINC02181 | LINC02182 | NONO | LOC101927727 | MIR3183 |
| NID2 | LOC101927839 | METTL16 | LINC01996 | MT1E | VENTX |
| MYCBPAP | SLC9A9 | ATP6VOD2 | NAT8B | C6 | PALLD |
| LOC101928266 | LINC00670 | RBBP7 | NEXN | HORMAD2 | SIGLEC9 |
| CDH3 | ICA1L | VLDLR | LINC01563 | PYY2 | FBXO42 |
| PIMREG | APOC1 | MOCOS | FBXO36 | CASC15 | RGN |
| FAM9A | LOC100505782 | PMEPA1 | C4BPA | MIR2117HG | LINC00473 |
| FAM153CP | RBPJ | APOBEC3C | PICART1 | TMEM92 | TMEM92-AS1 |
| CACNA1G-AS1 | CRYGN | ZNF474 | TBC1D3P1- | MAP1LC3B2 | C7 |
| | | | DHX40P1 | | |
| MNS1 | STMN1 | LOC101928021 | ECM2 | FHL5 | LINC01028 |
| SEC22A | LUM | KLHL4 | COL5A1 | SFT2D2 | LOC105373311 |
| L3MBTL4-AS1 | LINC01887 | LINC01882 | LINC01915 | LINC01894 | LINC01543 |
| MAP3K5-AS1 | LOC105372069 | SLC14A2-AS1 | TAAR9 | SPACA6 | ACSM1 |
| PDPN | LINC01415 | LINC01538 | LOC339298 | LINC01879 | LOC284240 |
| MAGEA1 | SLC4A3 | COL12A1 | SPTLC1 | LOC100996351 | SERTM1 |
| CTU1 | SVOPL | IGFBP5 | SLC9A7 | PLAAT5 | FMO6P |
| CD101 | LOC284454 | ABO | SPTY2D1OS | STEAP2 | SMIM33 |
| BISPR | CPXM1 | UNC13A | CCN5 | PNMA6F | ZNF626 |
| ZNF714 | LOC101929124 | LINC01785 | LINC01532 | SCUBE1 | ZFPM2 |
| MS4A10 | CACHD1 | SUPT20HL2 | STK11IP | MACROD2 | GLTPD2 |
| SMIM14 | LCN12 | APOC1P1 | TSGA13 | PNMA8A | SNAR-E |
| SEC1P | COL3A1 | DNER | RPS4X | NTF3 | NIBAN2 |
| ADAMTS6 | PGR | VSTM4 | ZIM2-AS1 | H3-2 | SAXO1 |
| HAND2-AS1 | MS4A8 | LINC01804 | CDH11 | LOC100287387 | HOXC5 |
| PTCH2 | CENPA | ENG | MS4A3 | EPYC | CCDC36 |
| LINC01460 | LINC00211 | RMDN2-AS1 | LINC01883 | CXorf38 | LINC02580 |
| METTL11B | MAGEB10 | CDS1 | LINC01185 | MEIS1-AS3 | LINC01797 |
| ASXL3 | RAB33B | MSC | ALMS1P1 | MEX3B | FOXL2NB |
| LOC102724579 | RAB11FIP2 | LINC01127 | PGM5P3-AS1 | CCR4 | ACTR3-AS1 |
| LINC01101 | RIT2 | EFCAB6 | NOC2LP2 | LINC01876 | LHX9 |
| REM1 | ISLR | SCN7A | LINC01960 | FAM153B | LOC100130691 |
| C20orf141 | SYNC | SPATA7 | CTAGE15 | HAND2 | FCN2 |
| SLC7A14 | LINC00607 | LINC00346 | TMEM198 | NLGN4X | GRIP2 |
| LINC01940 | MGC16025 | LOC200772 | ZG16 | RBP1 | BOK-AS1 |
| LOC285095 | TMEM239 | BLVRA | SNAP25-AS1 | KRT35 | MACROD2-IT1 |
| LOC613266 | SNORD17 | BTG1 | BRS3 | KIAA1671-AS1 | ABL1 |
| TMEM176A | MRC2 | DUSP11 | DLGAP4-AS1 | PRDX4 | SPRY2 |
| LOC149684 | KCNK15-AS1 | ATRNL1 | ZSWIM3 | LINC01754 | RBP5 |
| LINC01522 | LINC01523 | LINC00494 | CROCC2 | ABCA9 | APCDD1L-DT |
| LINC01711 | PIK3C2G | KRT31 | KRT19 | KRT36 | GSTM5 |
| ZBTB46-AS1 | SLC25A42 | LINC01697 | SMYD4 | SERPINH1 | C21orf62-AS1 |
| TCEAL3 | LINC01436 | KRTAP2-3 | ANXA13 | FAM153A | THEM5 |
| LINC00334 | COL18A1-AS2 | ST13 | LINC01659 | NOB1 | SLC5A8 |
| LRRC61 | ASAP3 | EPS8 | LINC02554 | LIF-AS1 | PRSS35 |
| HOXD3 | LL22NC01- | KCTD17 | LHFPL6 | LHFPL2 | ANKRD29 |
| | 81G9.3 | | | | |
| SULT1C4 | MRTFA-AS1 | XPNPEP3 | LOC105373044 | MROH2B | RRP7BP |
| EFCAB6-AS1 | LOC284933 | INKA1 | SRGAP3-AS3 | LINC00922 | HOTS |
| GPRACR | LINC00691 | POLD2 | LOC101927854 | RBMS3-AS1 | B3GLCT |
| COL14A1 | CFAP299 | DAPK1-IT1 | MIR4516 | C9orf47 | ADGRA2 |
| PRKAR1B-AS2 | MTFP1 | MYRF-AS1 | CDKN2C | RXFP2 | FCN1 |
| HAPLN1 | PRICKLE2-AS2 | ADAMTS9-AS1 | ZFPM2-AS1 | PDZRN3 | LOC101927296 |
| SAMD8 | LINC00879 | FCHSD2 | LOC101929411 | LINC00635 | TNP1 |
| LOC105374042 | ZNF804A | ZBTB20-AS4 | IGSF11-AS1 | TRIM72 | CCDC37-DT |
| MIR193B | PADI4 | BPESC1 | C10orf105 | ADRA2A | TM4SF1-AS1 |
| DCAF12L1 | PCDH18 | KCNE5 | ITLN2 | CPSF4L | BAMBI |
| LINC01209 | LINC00578 | EHD2 | PLA2G2E | FLJ42393 | LPP-AS1 |
| DCTN6 | LINC00885 | TM4SF19-AS1 | DARS-AS1 | LOC100130872 | SDS |
| LOC101928279 | LINC02517 | LINC02360 | GLI3 | KCNIP4-IT1 | TBC1D19 |
| ANXA5 | SP4 | LNX1-AS1 | LOC100506444 | SOBP | LINC02260 |
| UBA6-AS1 | C10orf82 | DNASE1L3 | CLSTN2 | GOT1L1 | EFHD1 |
| BMPR1B-DT | FLJ20021 | PIWIL1 | SUPT20HL1 | CXXC4-AS1 | SH3D19 |
| STON1- | ODF1 | TPPP2 | PDZD8 | SLC17A8 | RGS13 |
| GTF2A1L | | | | | |
| PP12613 | LDHC | EIF2B3 | LINC01197 | BRWD3 | LOC729870 |
| SIMC1 | CAPN8 | LINC02362 | POR | UBXN8 | LINC02436 |
| PLIN4 | SLC18A2 | LOC339975 | LINC01262 | CTD-2297D10.2 | LINC02217 |
| EIF3M | EGFLAM-AS2 | LINC01265 | IRS4 | SH3PXD2B | HPCAL4 |
| INSL3 | LOC102467080 | SCARF2 | LINC02225 | C5orf64-AS1 | STAMBPL1 |
| IL13RA1 | LOC102503427 | WTIP | CKMT2-AS1 | CSDC2 | MGC32805 |
| LOC101927357 | LOC105379152 | WWTR1-AS1 | ZNF774 | LINC01843 | FBXL21P |
| LOC100130172 | IFT57 | SCG2 | ERC2 | LVRN | LOC102546294 |
| TCF23 | ZPBP | FGF10 | FGF7 | GATA6 | LOC100507387 |
| LOC643201 | CD248 | MKX | LOC202181 | EFHC2 | MACC1-AS1 |
| CCNYL6 | IPPK | LINC00581 | LOC105374972 | LUC7L2 | SFRP1 |
| GUCY2F | MEDAG | DES | LINC01276 | HOXC6 | ARSB |
| RNF150 | FOXC1 | CCDC73 | GSTM2P1 | WDR64 | LINC01013 |
| TARID | LINC01312 | CT69 | LINC01010 | LOC644135 | LINC02539 |
| LINC01625 | UST-AS1 | BCL7A | LOC102723831 | SERPING1 | BORCS7 |
| SYNJ2-IT1 | LOC729681 | RPS6KA2-AS1 | HAS1 | LINC02538 | LOC105378137 |
| LOC105378146 | LOC101929460 | LINC01615 | LOC101929523 | HOXC8 | NYNRIN |
| KRT13 | INHA | NR2F1 | S100A6 | GPRC5A | ITIH6 |
| TYW1B | TPSG1 | NPAP1 | PMM1 | IDO1 | MFFP2 |
| NECAB1 | ZNF280C | PRDM6 | HSDL2 | GMPR | RCC1L |
| ENDOD1 | OTOR | C20orf203 | LOC105375115 | LOC101927769 | P2RY1 |
| PLAC1 | FAM170B | UPK3BL1 | LOC401324 | BMP3 | LCN9 |
| YWHAEP1 | LOC641746 | R3HDML | EGOT | DPT | COL1A2-AS1 |
| ZNF516 | LOC101927550 | ZFP36 | OMD | RGS2 | SGCG |
| PABPC5 | SLC13A1 | SLC51A | KLHL13 | EPHA1-AS1 | LONRF2 |
| BAG2 | ANKRD55 | SMIM10 | LINC00208 | MCHR1 | LOC102725080 |
| IFNK | COL5A2 | TMEM95 | LOC100128993 | LINC02870 | LZTS1-AS1 |
| LOC100507156 | RBPMS-AS1 | CCDC71 | LOC101929622 | LOC101929268 | ZCCHC12 |
| LOC100507516 | TENM4 | PSKH2 | E2F1 | MIR2052HG | OSGIN2 |
| MAFB | LOC102724804 | LOC101927066 | LINC02880 | LINC01181 | TMEM75 |
| GJB1 | FNDC10 | FJX1 | PTPRD-AS1 | ADAMTS18 | C9orf92 |
| DUPD1 | ATP8B4 | LOC101928195 | FAM27C | LOC102723709 | FAM27B |
| MAMDC2 | MAMDC2-AS1 | SMC5-AS1 | SPIN4 | PLEKHH2 | LINC01507 |
| LOC101927502 | SPATA31D5P | LOC440173 | MIR6081 | LOC100507346 | LINC01391 |
| TMEM246-AS1 | LINC01492 | HAMP | MTSS2 | ZDHHC8P1 | CENPH |
| C5-OT1 | SYCN | HTRA1 | LOC101929116 | PRRX2-AS1 | TRUB1 |
| DAPL1 | COL5A1-AS1 | LOC101448202 | PPP1R26-AS1 | PLA2G2F | CTXN1 |
| EML5 | LPAR4 | RFPL4B | VENTXP1 | RCAN2 | NDP-AS1 |
| ZNF469 | RASSF9 | LINC00269 | FOXS1 | GPC4 | RHOXF1-AS1 |
| IL1RAPL2 | KLF2 | WASIR1 | C8orf49 | IATPR | LINC02691 |
| LOC107984341 | SLC44A3-AS1 | DEPP1 | CLDN11 | WNK3 | FZD2 |
| LIN7C | TMC3 | CCL21 | PSAPL1 | ADRB3 | FAM110C |
| TNMD | FBXO43 | IGFBP4 | NBAT1 | ANXA2R | LINC02054 |
| STUM | ERICH3 | CREB3L2-AS1 | GRM7 | TMEM187 | CCDC80 |
| NRK | MIR12128 | C20orf85 | NAALADL2 | PLD6 | SLC2A7 |
| B4GALNT4 | C10orf55 | FBXL14 | GPR21 | RTN4R | B3GNT7 |
| ITLN1 | PROK1 | PNMA8B | CHAF1A | HSPB2 | C9orf147 |
| IL20 | MYL9 | PIRT | CCDC26 | LINC00319 | ELF3-AS1 |
| CARMN | C2orf91 | C10orf142 | GLRA4 | COL6A1 | HS3ST1 |
| KLHL15 | LOC284898 | | | | |

Pancreas

| | | | | | |
|---|---|---|---|---|---|
| FTO | SPSB4 | NAP1L2 | MIR4251 | RUVBL1 | TUSC3 |
| AFP | THOC6 | RBMXL1 | CHN2-AS1 | MANEA | SYCP1 |
| SCFD1 | FGL1 | C1orf195 | KLHDC7A | IZUMO1 | MST1P2 |
| ST18 | AKR7L | PON3 | KANSL1 | LINC01635 | SLC25A45 |
| ZBTB40 | WDFY2 | THAP12 | ZNF436-AS1 | NIPAL3 | GLRB |
| TBILA | PREPL | GATC | GATA4 | KRTAP1-3 | SNTG2 |
| TMEM212 | TRAP1 | YTHDC1 | PDE6H | PABPC4 | WNK2 |
| MYO1B | OCLN | CASP9 | GIT2 | EML4 | PPIEL |
| TRIT1 | CLLU1 | SP8 | TRAPPC4 | GPRC5C | DPP10 |
| MSI1 | RAP1GAP | HELZ | MFSD6L | CACNG1 | ASB11 |
| MKNK1-AS1 | BTF3L4 | TRIB3 | NPNT | MTRF1 | ROMO1 |
| PEX5L | EIF2A | FAM221A | STK31 | LINC01748 | ZFP91 |
| BACE1 | SRP19 | SH3BP4 | MGAT5 | MPV17L | MTHFD2L |
| MKNK1 | POU2AF1 | GNG12-AS1 | ADIRF | CEP112 | ZRANB2-AS1 |
| CPA3 | C1orf194 | ELOVL7 | SLC29A2 | LOC389831 | MAGI1-IT1 |
| PKN2-AS1 | SEC31A | GBP6 | CLU | ANAPC10 | CHKA |
| C1D | KANK4 | C1orf116 | HNF4G | PNPT1 | NRCAM |
| TMPRSS2 | RTF1 | VEGFA | SSTR1 | ACTG1P4 | KIAA1324 |
| RASSF10 | MCCC1 | SIGLEC11 | TRIM46 | PCMTD1 | UPK3BL2 |
| ZNF57 | RAB39B | GCAT | POU5F1 | ATP1A1-AS1 | ARHGEF38 |
| NMD3 | COBLL1 | LOC101929798 | LRMP | IL33 | CCDC186 |
| TMEM98 | CEP70 | TAFA2 | CD24 | PTENP1 | NIP7 |
| BCL10 | UGT2B10 | SEC23B | ACMSD | ACSS1 | CBY3 |
| PLA2G10 | KLK15 | ACKR3 | NRG4 | DENND1C | EHBP1 |
| MYO5C | INTS6 | ESRP1 | CCDC34 | B3GALT5-AS1 | DIAPH3 |
| LOC101928565 | LINC00970 | ATXN7 | SUCO | SNORD80 | LOC101928696 |
| NOMO3 | ACP1 | OR2H1 | CLPSL1 | SERPINI2 | HES5 |
| ARHGAP24 | MEIOB | PPP1R14A | CTH | SWT1 | GS1-279B7.1 |
| SRPK1 | KCNAB1 | HNF1B | HSPB3 | PIWIL2 | RASSF6 |
| SORBS2 | TNNI2 | LOC284581 | WASF1 | TRIM73 | UBE3D |
| MKRN1 | ECE2 | LPGAT1-AS1 | RNF212 | PROX1-AS1 | LARP1B |
| KLK13 | KIRREL2 | SGO1 | SFTA2 | SLC30A6 | LINC01682 |
| SLC26A3 | STAP1 | COBL | SLC22A9 | UBA6 | CHRM3-AS2 |
| CXADR | GLYAT | PACSIN2 | HYOU1 | AKR1C6P | ANXA4 |
| FAM13A | LINC02656 | SPP2 | CACNG5 | CHST4 | IGFL2 |
| FZD4-DT | KIF14 | BMP2 | ARMC3 | FANCL | CA12 |
| GDAP2 | CNPY1 | PM20D1 | LINC01517 | GRIA4 | LRRC17 |
| GAS2 | MTUS1 | LINC01518 | AMHR2 | RSU1P2 | BANF2 |
| SHLD2P1 | CTSLP2 | COX11 | BRSK2 | SLC33A1 | SLC16A7 |
| ZKSCAN1 | ZNF281 | MAP1LC3A | OXSM | PLEKHS1 | WLS |
| MRPS35 | C6orf141 | EDEM3 | AP2A2 | GZMB | SLAIN1 |
| C12orf45 | SPON2 | IPO8 | MNX1-AS1 | BTG3-AS1 | DRC7 |
| LRMDA | LOC105378367 | KCNMA1-AS3 | SERAC1 | MECOM | BMS1P21 |
| BCL2L15 | EVA1A | LRIG3 | PEX11A | DHRS4L1 | FLJ37201 |
| TMEM60 | COCH | ANO5 | RAB27B | RBP2 | IFT20 |
| C2orf72 | DYRK3 | SLC41A2 | VTCN1 | IFRD1 | LRRC6 |
| EGF | CXCL5 | SOAT1 | HNF4A-AS1 | KRTAP5-10 | CDKN1A |
| GLP1R | NR2F2 | COMMD8 | ARL14 | AGTR1 | FITM2 |
| SLC39A14 | SERPINA4 | BCAT1 | LINC00339 | SHLD1 | KISS1R |
| MFF | CAPS2 | EDRF1-AS1 | CRISP3 | FANK1-AS1 | NUDT9 |
| GNB5 | NR5A2 | CRACR2B | PROX1 | MRPL23-AS1 | IGSF11 |
| LEMD1-DT | PAK3 | COPB2 | NLRC4 | RAPGEF5 | LOC102724784 |
| LOC105378979 | SLC22A24 | TFPI2 | GARS1 | KLHDC2 | CACNB2 |
| UGT2B7 | NAV2-AS4 | CLDN2 | SARM1 | NAA16 | MGAT4C |
| GRB10 | KLF5 | ENPP1 | CLDN10 | TMEM51 | CADPS |
| NEK2 | BCCIP | CHN2 | OOSP1 | NUDC | RECQL |
| SLC16A10 | VRTN | LINC02418 | LINC00673 | SLC38A11 | HBP1 |
| C4orf47 | INSIG2 | GLS | MRPL49 | CD84 | AFM |
| DIRAS3 | TEX26 | MARVELD2 | TRIM2 | CLDN9 | B3GALT1 |
| KCNJ13 | NADK2 | FEV | PCK2 | CYLC2 | MTHFD2 |
| DNAJB11 | THNSL1 | ACBD6 | TMEM97 | ZNF22 | HOMER2 |
| LOC646029 | F8 | CCDC141 | ZNF711 | SPATA31C2 | KPNA1 |
| RORC | ZC3H7A | LOC101928535 | COLCA1 | COLCA2 | C19orf81 |
| UBE2F | CCDC151 | LOC100652768 | APOBEC3B | RNF214 | TMEM125 |
| FIG. 4 | SLC28A3 | ALB | C11orf45 | SNAI2 | TOM1L1 |
| UGT2A3 | KDM8 | ATP5MC1 | DCUN1D5 | NIBAN1 | AJUBA |
| CBS | CCDC172 | CTNND2 | MCIDAS | ARMCX5 | GPR39 |
| TSSK4 | KLK12 | MGAT4A | GHRLOS | EPCAM | TPST2 |
| ENOPH1 | TPD52 | DSTN | PTX3 | IGFBP2 | CCDC196 |
| KRT7-AS | KCNE3 | KCNU1 | TMEM198B | GHRL | FBXW12 |
| A1CF | ACADL | ARHGEF3 | FOXL2 | G6PC | ELL2 |
| CALCA | PTGER4 | ERLEC1 | TBC1D30 | LINC02389 | SPATA13 |
| LINC02421 | STEAP4 | PRDM16-DT | MRS2P2 | TRHDE-AS1 | ANKRD7 |
| CCDC65 | LRATD1 | NLK | LRIG1 | ADH7 | SMIM20 |
| MKRN9P | ABAT | PTGER3 | PGAM1P5 | LOC643711 | SLC9A7P1 |
| LINC02453 | CXCL11 | SCYL2 | GNL3 | TMCO5A | SCRN3 |
| SMIM28 | GIN1 | LMAN1 | CEACAM7 | RBP4 | NRAV |
| BCL2L10 | BBIP1 | STXBP6 | LMBRD1 | SLC4A4 | SLC49A4 |
| LINC02347 | LINC02405 | RASD1 | C10orf67 | SMIM18 | SERPINA3 |
| SELENOM | USP12-AS2 | PLUT | LINC00543 | LINC00572 | TEX26-AS1 |
| FRY-AS1 | NDUFV2 | IRF6 | STT3A | TPTE2P5 | LOC101929140 |
| FOXA1 | VSX1 | KLF15 | GALNT11 | LINC00562 | DHRS12 |
| C12orf73 | RNF148 | CXCL6 | CLDN10-AS1 | DNAJC3-DT | DOCK9-AS1 |
| ECI2 | GGACT | H1-8 | ERN1 | SNHG3 | NXPH2 |
| TFF3 | APOH | TRIM32 | DHRS4-AS1 | KIAA0895 | FGF21 |
| LOC100506071 | MAP3K20 | PDIA4 | MIPOL1 | TCAIM | CYP2C8 |
| PRR23C | SEC61B | KANSL1L | OTX2-AS1 | AQP5 | SNRK |
| MBLAC2 | ING2 | IL18 | GRIK1 | TFEC | LOC145474 |
| EGR4 | PSAT1 | BPHL | FLRT2 | ZBTB18 | FOXN3-AS2 |
| MPC2 | GSTM3 | LINC02287 | DNAJB4 | LOC107984703 | LINC00239 |
| SPIN2A | GRB14 | CPNE6 | FCF1 | MMADHC | BOD1L2 |
| RPH3AL | LINC00929 | HERC2P10 | INSR | STK17A | SRP14-AS1 |
| RWDD1 | PWWP2B | ANAPC5 | LINC01491 | AP3S1 | ONECUT1 |
| RORA-AS1 | CFTR | LOC107984784 | APH1B | URAD | ANAPC16 |
| ANGEL1 | IQCH-AS1 | GGCT | DRAIC | RASEF | CNTD1 |
| PELI2 | ODAM | MS4A2 | FANCC | CCDC181 | MAP3K21 |
| FIGLA | TMED3 | ZNF710-AS1 | VPS33B-DT | PCBP2 | LINC02254 |
| RAB1A | LOC105371038 | XPOT | GNPDA2 | DANCR | AKR7A3 |
| MCCD1 | LOC100128770 | STEAP1B | B3GNT2 | TC2N | CNN3 |
| YIPF4 | BFAR | C9orf135 | FBP1 | GPX2 | EHF |
| INTS12 | KCNG3 | SLC2A2 | ETV5 | GAD2 | GLIPR1L1 |
| PPP1R10 | ANKRD31 | ABCE1 | UMOD | PYCARD-AS1 | MYOM1 |
| GDF6 | DPY19L2P3 | CASC16 | GP2 | NKX2-4 | B3GALT5 |
| SPAG17 | ABHD13 | ARL8B | MAFTRR | TLCD4 | RNF133 |
| LOC102724467 | LINC02091 | AMER3 | LOC101927911 | LIPJ | TKTL2 |
| DLD | EIF2S1 | MIR27B | MAGOH2P | GMNN | SEL1L3 |
| LINC01992 | KRT18P55 | MRPL33 | AOX1 | IFITM2 | ARGFXP2 |
| SPACA3 | GRPR | AZIN1 | SYNPR | AOAH | ID1 |
| PEX6 | AOC4P | ARSL | LGALS3 | SDR42E1 | SOX9-AS1 |
| B3GALNT2 | KLK11 | DUSP4 | KCNE4 | GLYATL3 | PARD6B |
| FABP3 | F11 | MTUS2 | CCDC110 | LOC105371880 | CASC17 |
| LINC01152 | LINC02097 | LINC02003 | CXCL9 | LINC00469 | NRTN |
| ZFAS1 | CBLIF | RBPJL | DNAJC3 | TMA16 | APOBEC2 |
| NRAP | GAMT | MARCKS | MIR4740 | LINC02735 | PIP5K1B |
| KCTD16 | CXCL12 | LINC00470 | DLGAP1-AS1 | TEX11 | LOC101927188 |
| SLITRK1 | PTH2R | TIFA | GCSAM | ACSM1 | LOC642484 |
| SLC3A1 | KCNJ5 | LINC01910 | LINC00683 | EPOR | HSPA9 |
| NSF | LOC100128568 | MINAR1 | PRODH2 | IMPG1 | UGCG |
| NPEPPSP1 | BEX3 | ZDHHC2 | TM4SF1 | CHRM1 | AMACR |
| DEUP1 | LINC00641 | LOC101929124 | LOC102724908 | ARSH | GSTA5 |
| NMNAT3 | DERL1 | LINC01529 | SV2C | AKAP9 | DDC |
| FBLN5 | SPAG9 | SMIM14 | GYS2 | CCDC105 | TMEM64 |
| TRAF5 | FAM47E | TBC1D8B | ATP1A1 | LAMA1 | LINC01869 |
| PKHD1 | NOMO2 | LMNTD2 | NPEPPS | SNTG2-AS1 | LINC01247 |
| LOC101929551 | MS4A8 | RAMAC | LINC01954 | ADH4 | STAT4 |
| NLRP9 | LHFPL3 | TMEM211 | LINC01320 | DDI1 | TRHDE |
| LINC01813 | MEIS1-AS3 | LINC01829 | LINC01812 | CLPSL2 | ERCC8 |
| OR7E91P | SNX3 | REG1CP | ANKEF1 | EML4-AS1 | ERLIN2 |
| MTCP1 | GTF2H5 | CCNYL1 | MSRB2 | CFAP221 | LOC101927881 |
| LINC02572 | LOC440910 | LINC01945 | LYPD6B | CSMD3 | LINC01876 |
| C10orf120 | IFNL4 | INPP4B | LOC101929633 | ALDH1A1 | LSMEM1 |
| AGR2 | NFKBIZ | LOC105373878 | ASZ1 | ARF6 | LOC646736 |
| ANKRD62 | DAW1 | OMA1 | LINC00471 | MYOZ2 | KCNJ1 |
| C4orf33 | LINC01237 | LINC01238 | LOC285097 | LINC01881 | NPY1R |
| MIOX | SNAP25-AS1 | GDF15 | SPAG16 | LOC284788 | GDI2 |
| ANXA10 | LOC100130373 | BMT2 | LOC101927159 | LOC101927138 | LOC100131496 |
| SI | PNMA1 | RNPC3 | GRIK1-AS2 | GRIK1-AS1 | FBXO22 |
| SEC14L3 | LOC101928269 | LINC01436 | SRP54 | LEAP2 | UGT8 |
| LINC00479 | UMODL1-AS1 | CYSTM1 | LINC01669 | NHLRC2 | PDE7A |
| LOC101928796 | HSD17B14 | SLC16A1-AS1 | FOXA3 | EPRS1 | B3GALT1-AS1 |
| B3GNT5 | AGBL3 | ADAM7 | LARGE-AS1 | DNASE2B | PRSS22 |
| GSTCD | SLC15A2 | CRNN | LOC100130207 | VNN1 | POU1F1 |
| PLCL2 | RHCG | FANCF | LGR4 | KDM3A | SAAL1 |
| LINC02685 | MOXD2P | ENTPD3-AS1 | FSCN2 | CCDC13-AS1 | CCZ1P- |
| | | | | | OR7E38P |
| CHMP4A | NRADDP | GSTA1 | PF4 | MTMR9 | UGT1A6 |
| ACTN2 | ERC2-IT1 | COL19A1 | IFNLR1 | LINC00698 | PSMD6-AS2 |
| MAGI1-AS1 | SLC25A26 | SMIM8 | OVAAL | FOXP1-AS1 | SP9 |
| RNLS | NCMAP | NDUFA6 | PDGFA | CELA3B | NUAK2 |
| NLRP11 | RUVBL1-AS1 | DNAJB8-AS1 | CELA2B | IMPA2 | SLC22A31 |
| CHRNA9 | BPESC1 | SRBD1 | JCHAIN | ADRB1 | ADRA2A |
| LINC01213 | KCNAB1-AS2 | SLC17A2 | PRKCA | MACIR | CPSF4L |
| PGGT1B | YEATS2-AS1 | LINC02069 | GALNT3 | LINC02052 | LINC02038 |
| LINC00885 | ALDOB | ALAD | CERS6 | LOC105374344 | TMED11P |
| USP2 | CHAD | LOC439933 | VSTM5 | HBZ | TAGLN |
| IRS1 | USP46-AS1 | HCN1 | RXYLT1 | LOC105377267 | CDX2 |
| TBX10 | DCLRE1A | LOC100507053 | LOC102723704 | PLA2G12A | KLF14 |
| WSPAR | LOC101929762 | SLC7A11-AS1 | KLKB1 | F2RL2 | LINC02266 |
| SMAD1-AS2 | NPHS1 | DZIP1 | LINC02864 | FAM187B | CHST7 |
| LOC100996325 | OLFM4 | LINC02150 | SFRP5 | LOC101929645 | LINC02104 |
| FBXW8 | MESTIT1 | MIR193A | LUADT1 | ARF4 | LINC02101 |
| LINC02057 | RNF139 | PRRC1 | LOC441204 | LOC101927078 | TTYH1 |
| LOC105379143 | KCNK5 | ADAMTS19- | DCDC2 | LOC100130172 | FAM161B |
| | | AS1 | | | |
| BDKRB1 | FZD5 | PSD2-AS1 | SKA1 | SMPDL3A | MAT1A |
| ALDH1L2 | TCN1 | STK32A-AS1 | NMUR1 | HSPA13 | SCGN |
| MIR3142HG | KCNH7-AS1 | GATA6 | NOMO1 | C2CD4A | XIRP2-AS1 |
| ELOVL2-AS1 | RNF208 | GOLPH3L | C8orf37 | GCNT4 | PSORS1C3 |
| ENPP4 | PDILT | LOC100507547 | LOC100294145 | IL22RA1 | GPR62 |
| LINC00951 | TSPO2 | PPIF | TSPAN13 | EME1 | C15orf54 |
| LOC101926898 | MUC6 | CFAP52 | GSTA7P | LGSN | SYCP3 |
| PDX1 | CLDN1 | PTP4A1 | TFF1 | LINC02532 | OSTM1-AS1 |
| LINC00222 | BRD7P3 | LINC02523 | LOC101928140 | CD80 | TAAR3P |
| LINC01010 | DDX4 | LINC01625 | LOC103352541 | GFPT1 | NTN4 |
| TMEM65 | STC2 | CTTNBP2 | APOC3 | PRSS16 | INHA |
| LRG1 | TSPAN12 | TNKS | ZBED6 | HNF1A-AS1 | MPP6 |
| UBE2R2 | POF1B | CPQ | PRLHR | SCNN1G | EPO |
| GPX1 | CHRNB3 | ABCC12 | LOC102723672 | HRAT92 | GLUD2 |
| C6orf201 | SLC30A2 | EFHD2 | CTRC | GPR63 | SNHG26 |
| KSR2 | LOC105375218 | TMEM163 | PIGB | POU6F2-AS2 | KRTAP1-1 |
| C7orf25 | LDHB | PRSS3 | MEIS1 | DDC-AS1 | FKBP9P1 |
| CCT6P3 | INAFM1 | GTF2IP7 | ARL1 | DPT | PPP3R1 |
| C4orf48 | HID1 | NOCT | LOC105375423 | VAMP5 | PNLIPRP3 |
| C11orf91 | LOC101927870 | LHFPL3-AS2 | AQP8 | LINC01393 | MAL2 |
| SLC13A1 | LOC105375504 | PRSS37 | OR2A1-AS1 | PRKAG2-AS1 | ATP4A |
| NFE2L3 | LINC01022 | FAM87A | LOC101929269 | TDH | FAM167A-AS1 |
| EVI2A | TMEM258 | FAM162A | UGGT2 | H2BW2 | LINC02870 |
| LINC01355 | RNF5P1 | SLC10A5 | TSPAN8 | LOC401463 | MSC-AS1 |
| STAU2-AS1 | MPZL2 | PUM3 | AASDHPPT | LOC100288748 | LINC01298 |
| LINC01181 | C4orf19 | PIK3AP1 | ERMP1 | MAL2-AS1 | LOC101927543 |
| LOC101927588 | LOC101927798 | MYH7 | GJB1 | PCSK1 | PCDHB1 |
| TESC | MIR429 | GLIS3-AS1 | SLC7A2 | CELA2A | EIF4EBP1 |
| LOC102724238 | LOC642929 | SELENOK | CLDN6 | SRPRB | LINC01474 |
| KCNK3 | LOC101927623 | GAS1RR | LINC00484 | PCAT7 | BARX2 |
| PTCSC2 | ANKMY2 | FICD | LNX2 | SYCN | PRSS8 |
| LOC105376271 | LOC51145 | DSG2 | GUCY1A2 | DDX1 | CELP |
| PGRMC2 | MAN1A2 | SEL1L | GUCY2C | ERO1B | CD99P1 |
| MAP3K5 | LGALS4 | FAM3B | ZFPM1 | SPESP1 | RNF144B |
| ROS1 | LINC00630 | SOWAHB | PLS3-AS1 | PCAT18 | CATSPERB |
| APLP1 | SOCS1 | CELA3A | MKRN3 | CDC42EP4 | C8orf49 |
| ARMH2 | MST1L | LOC339260 | CCDC173 | FAM81B | TMEM236 |
| MGAM2 | OR9Q1 | LOC644090 | SERPINE3 | CBWD6 | BPIFA3 |
| SDC4 | FAM110C | CCDC158 | EN1 | UTRN | ITPRIPL2 |
| SCGB2B2 | CEACAM16 | UCHL1 | WHAMM | LURAP1L | ZACN |
| DNAH8-AS1 | BICDL2 | MRO | PRR27 | KLB | GPR148 |
| RDH12 | KRT7 | B3GNT7 | SEC63 | AMIGO3 | HOOK1 |
| ATOH7 | GDF10 | PARM1-AS1 | LINC00261 | PDE11A-AS1 | PBX1-AS1 |
| H3P4 | LINC02869 | SIRT4 | ATP11AUN | LOC107985770 | ANPEP |
| AADACL2 | ATP6V1G1 | FNDC7 | GSTA2 | SIM1 | TMEM181 |

Prostate

| | | | | | |
|---|---|---|---|---|---|
| PDE9A | ZNF532 | LOC100129534 | TRPC4 | LOC101928764 | RCAN3 |
| ISX | ADPRM | C2orf80 | TRPM4 | TMEM45B | AKAP1 |
| NIPAL3 | ZNF385B | ORM2 | TMEFF2 | CYP3A43 | CAPG |
| SPDEF | SUPT3H | SLC44A4 | NEDD4L | BMPR1B | HOXD8 |
| EVX1 | SENP8 | SLC39A6 | CCDC178 | MALT1 | C1D |
| POTEM | C1orf116 | TMPRSS2 | EDNRA | CCNQ | BEND4 |
| ARHGEF38 | CWH43 | GNRHR2 | CHRNA2 | PTCRA | PARP11 |
| CCK | MEP1A | TMEM79 | GLMP | POTEJ | LOC101928372 |
| OR51E2 | PACRGL | THSD4 | SRRM1 | HHLA2 | PPP2R3C |
| LOC148709 | ABCC4 | FAM201A | RAB27A | LOC100287497 | SLC26A3 |
| HOXA-AS2 | MARCHF10 | DCLRE1C | C22orf42 | RORB | CALML3-AS1 |
| MME | LOC102724323 | AGAP4 | CLDN8 | HNRNPA1P33 | AGAP12P |
| ANTXR2 | ACER1 | PLEKHS1 | SLCO5A1 | SUV39H2 | FAM13C |
| SNAPC1 | SLC2A10 | CDC45 | DYTN | MKI67 | LINC00974 |
| RAB4A | ACP3 | GP6 | CCNG2 | MYBPC1 | TEX36-AS1 |
| COQ7 | ST8SIA1 | CLNK | MRGPRG-AS1 | HHIPL2 | DIO2 |
| JPH1 | IL13RA2 | SERPINB11 | ERI2 | LOC101928894 | FLNB |
| OPRK1 | PI15 | SLC2A12 | LANCL2 | DRD1 | SSTR5 |
| KLK4 | LOC101929295 | OAZ3 | COLCA2 | C11orf88 | C16orf54 |
| FBXO34 | MUC3A | TOM1L1 | ZNF827 | NCEH1 | KLK2 |
| MED9 | LOC440084 | SLC25A37 | BBS4 | CDH26 | SLC22A3 |
| PRR11 | MRPL35 | STEAP4 | STARD3NL | NEFH | PRKG2 |
| CABCOCO1 | LOC643711 | CLDN12 | ARMC4 | TXNDC2 | NAAA |
| LINC00507 | PPP1R7 | RDH11 | ZNF350 | LINC00415 | LINC00572 |
| MCM8 | C13orf42 | PIP4P2 | LINC00558 | CXCL6 | P3H2 |
| KLHL14 | PACSIN1 | TTK | LINC00343 | LINC02297 | POTEG |
| BMS1P22 | LOC100508046 | PRDM8 | HRH3 | MIPOL1 | CASZ1 |
| ARL14EP | LRRC9 | ARFGAP3 | DIO2-AS1 | LINC01467 | RSPH1 |
| MARCHF5 | MEAF6 | NPM2 | ISL2 | LOC145845 | PARP12 |
| KRT23 | TARP | AGAP11 | DRAIC | TMPRSS11E | GREM1 |
| C6orf52 | TMEM220 | ISY1 | EFL1P1 | EIF3H | ASB9P1 |
| LOC101927751 | RAB6A | CTBS | HOXA5 | EHF | MCCC2 |
| PROSER2 | RNF186 | DESI2 | COPRS | KRT17 | GP2 |
| ILRUN | STYK1 | POTEF | ZNF789 | NAT1 | LINC01229 |
| PCCA-DT | RPL29P2 | SLC14A1 | FAM135A | GMNN | ALDH1A3 |
| MC5R | FBXO36 | KRT42P | PMEPA1 | FAM153CP | ALOX15B |
| ACP4 | TFAP2C | MRPS23 | HOXB2 | RFX6 | CD177 |
| LOXL4 | ST8SIA6-AS1 | CD47 | LOC105371998 | KCNC2 | LINC01443 |
| LOC105372071 | TGM7 | OACYLP | LINC01927 | ZNF236-DT | PLA2G7 |
| DNAH8 | TEX36 | LOC100128568 | ZNF812P | FRMD4B | KRTAP10-2 |
| GOLM1 | STEAP2 | MIR200A | NWD1 | SAMHD1 | MFSD4A |
| THEG5 | LINC01533 | ABHD17C | TRPM8 | ADPGK | PLA2G4C-AS1 |
| KLKP1 | ZNF350-AS1 | EYA2 | TPM3P9 | HEPACAM2 | CSNK1A1L |
| NLRP8 | MALL | LOC105373352 | ODC1-DT | LINC01376 | PAK1IP1 |
| RPF2 | LG11 | ANO7 | TMEM211 | CFC1 | EPCAM-DT |
| PRM2 | CCDC15 | CYP4F11 | CPNE4 | LOC101927881 | FAR2P2 |
| CYP4F30P | RMST | POTEKP | LOC101929633 | LOC401021 | FAM153B |
| TRPV6 | OR51E1 | PHF21B | LMAN1L | MIPEP | NANS |
| SNORA71E | LINC01429 | LOC105372672 | ACAD8 | SLC45A3 | LINC01742 |
| KRT36 | OVOL2 | LOC101928107 | DSCR4 | DSCR8 | XKR5 |
| SPATC1L | SLC30A4 | LINC01668 | FAM153A | PARP8 | CES5AP1 |
| FAM2301 | MMP7 | B3GNT5 | AP1B1P1 | LINC01399 | DNASE2B |
| MPPED2 | LOC101927551 | LOC105373064 | LINC01644 | NANOGP8 | POTEE |
| TTC6 | SRGAP3-AS2 | POTEI | MCF2L2 | PLEK2 | LINC02050 |
| FSTL1 | KRT2 | GLIS1 | APIP | GABRG3 | ITLN2 |
| MRPS10 | LINC01983 | SIM2 | PRIM2 | LINC02261 | SRD5A3-AS1 |
| LOC101928809 | PCAT4 | DCLRE1A | BMPR1B-DT | GRHL2 | MRPL58 |
| TLR1 | ZG16B | POLR3K | CRNDE | SPATA12 | LINC02433 |
| SFTPA2 | LRRC42 | KL | LINC01093 | LINC01511 | LINC01019 |
| LINC02199 | LOC100130744 | ZDHHC18 | TLR6 | LUADT1 | ANO1 |
| LINC02065 | LINC02219 | GUSBP9 | PRRC1 | LINC02240 | LINC02169 |
| AGAP7P | TMEM238L | LOC105379176 | FAM161B | GPR160 | TCIM |
| ANKRD1 | LOC100996385 | LOC100507387 | LOC105377752 | LOC100129697 | LINC02222 |
| LINC01622 | TFAP2A-AS1 | LOC101928191 | LOC101928491 | GUSBP2 | LAMP3 |
| PDILT | MIOS | POTEH | GHRHR | IZUMO3 | LOC101927136 |
| ZNF235 | MCM2 | KLK3 | MSTN | AGPAT4-IT1 | LOC105378127 |
| KRT13 | RUNDC1 | PITX1 | ALOX12B | ADRA1D | ATP2C2 |
| MFFP2 | SRD5A2 | SCNN1G | CYP4F8 | KRTAP1-1 | RHPN2 |
| YWHAEP1 | GTF2IP23 | ARG2 | DPY19L2P4 | AZGP1P1 | TMEM210 |
| C8orf86 | KRT16 | OR6W1P | PCAT1 | LOC285889 | LINC01022 |
| KBTBD11-OT1 | GS1-24F4.2 | HOXD9 | LOC340357 | LOC101929066 | LOC105379393 |
| LINC00536 | FAM83A-AS1 | TDRD9 | NKX6-2 | LOC101929563 | HOXD12 |
| LINC00484 | LOC340512 | LINC01509 | ARFGEF3 | ZDHHC8P1 | TUBA3D |
| LINC01613 | CLDN3 | PM20D2 | ZNF614 | PTGS2 | KRT40 |
| FAM3B | ANKH | RGS10 | RGS1 | C11orf53 | LSM11 |
| LOXL1-AS1 | AGAP10P | PARGP1-AGAP4 | OTULINL | LOC105369201 | LOC339260 |
| LOC105377777 | SLC44A3-AS1 | LINC02688 | KRT14 | TMEM192 | WNK4 |
| CFC1B | TDO2 | OR13J1 | MAF | ZNF804B | ORC5 |
| SIGLEC15 | KRT39 | ASCL4 | BMP8A | GPR20 | DLEU7-AS1 |
| HPAT5 | GUSBP16 | DELEC1 | NCAPD3 | PRSS41 | HOXA11 |
| PCSEAT | HOXD10 | MLC1 | | | |
| Skin | | | | | |
| PAX3 | RAX | RGMA | TNXB | PEBP4 | ADAMTS10 |
| LMNA | HOXB6 | MMP11 | METRNL | HOXB-AS3 | LOC105378933 |
| TMEM151B | YPEL4 | C6orf132 | SLC52A1 | TEDC1 | CMKLR1 |
| PLAU | ADAM15 | PHLDA3 | NKX2-5 | DOK1 | MAP3K6 |
| LINC01744 | SBSN | PRRX2 | VSIG10L2 | C1QTNF1 | PLD4 |
| NT5C | KPRP | LOC105378269 | LINC01264 | LINC01605 | MMP2 |
| AHNAK2 | FAM170B-AS1 | SYTL1 | PLEKHM1 | NKX3-2 | AGAP2-AS1 |
| EMP1 | RTEL1 | LEXM | IQCN | KCNK4 | MFGE8 |
| LOC101927495 | PITX2 | TMEM200B | KRT1 | DNM1 | ABR |
| EDN3 | HOTTIP | ATP13A2 | FAM138D | LINC02449 | C1QTNF4 |
| F13A1 | TBX1 | TIMP1 | HOXC13-AS | FLJ12825 | AHRR |
| OSBPL5 | MEGF6 | CNRIP1 | FZD10-AS1 | LOC107986453 | FNDC8 |
| CYP11B1 | CLEC2A | PLXNA1 | BCL11B | HR | GPR153 |
| OGFRP1 | EGR1 | HSPA12B | CYP26B1 | GSC-DT | RASA3 |
| HOXC11 | LOC101929439 | HOXA11-AS | EMX2 | PRPH | LOC100288162 |
| LOC101928343 | ANXA8L1 | AHNAK | EVPLL | LOC101927793 | RIN1 |
| MYL5 | REEP4 | HOXB-AS1 | FLJ40194 | LOC100288866 | LINC01279 |
| TFAP2C | HIC1 | IFITM5 | LINC01443 | MMP14 | STMN3 |
| ARC | ANXA8 | PRLH | CD70 | NOP53-AS1 | MIR26A1 |
| GADD45G | SLC17A7 | LINC01865 | LINC01875 | CBR3-AS1 | COL5A3 |
| CYP11B2 | LINC01124 | TPTEP2- | RTN4RL2 | KCNA6 | CSPG4 |
| | | CSNK1E | | | |
| GOLGA2 | LOC101929413 | EMX2OS | MRC2 | IRX3 | LINC01260 |
| LOC100506175 | GSC | PICSAR | LIF-AS1 | ACAT2 | IRX1 |
| PPL | LINC00898 | LINC00691 | ADGRA2 | LOC284930 | RITA1 |
| LINC00696 | INHBB | HES2 | WDR34 | ESRG | FBLN2 |
| COLGALT1 | AADACL2-AS1 | HOTAIR | ADAMTS2 | LINC02493 | ATP6V1E2 |
| DBN1 | SLC25A48-AS1 | IBA57 | CCBE1 | LINC02116 | LINC02223 |
| LINC00514 | LORICRIN | CYGB | LOC101927969 | JMJD7 | LOC101928663 |
| PKP1 | LOC101929188 | TRIM29 | CLDN4 | LOC105378052 | PSCA |
| DNAI1 | FZD10 | ZNF703 | CYP1B1 | SLC35F6 | GARS-DT |
| TMEM30B | CITED4 | PTGIR | ST7-AS1 | LOC340357 | HS3ST2 |
| PTGDS | SCARA5 | TBX15 | TNK1 | LOC100128076 | TMEM275 |
| LINC01503 | PLA2G2F | CD164L2 | LINC00632 | LINC02783 | CDC14C |
| THBS2 | CCDC140 | TAS2R41 | C14orf177 | HS3ST6 | LINC00452 |
| IRX6 | LINC02874 | CXCL14 | | | |

Testis

| | | | | | |
|---|---|---|---|---|---|
| STOML3 | LINC01134 | LINC01777 | TLCD3B | SUSD4 | ST6GAL2 |
| TIAM1 | IGLL5 | ACTRT2 | TXNL4B | FABP6 | SLC30A8 |
| SERPINA12 | SLC1A6 | KRT75 | NR5A1 | SLC6A11 | SLC22A14 |
| CYMP-AS1 | INS | HSD17B3 | ZNF160 | LILRB5 | KRT38 |
| LOC101929798 | GHSR | PCDHB15 | PRSS1 | TSBP1-AS1 | ENPP5 |
| KCNS2 | LOC101928372 | APOBEC3H | WRAP53 | LOC100505918 | SIN3Bss |
| SALL4 | CRYZL2P | ZNF763 | MROH3P | PRAME | TBPL2 |
| LOC91548 | SLC7A4 | ZNF112 | CFAP53 | CCDC33 | LINC00200 |
| MUC21 | RACGAP1P | ZNF534 | PLPP4 | OR7A5 | ALLC |
| LINC02622 | SLC30A3 | IFT20 | CCL24 | TMEM63C | C4orf54 |
| LINC01168 | SLC52A3 | CLNK | ADGRE3 | OLFM5P | PNPLA5 |
| MPPED1 | CCDC168 | KLF17 | CBY2 | NEK2 | GNLY |
| BUD23 | EFCAB8 | TSGA10IP | NDUFAF1 | CYLC2 | LINC02878 |
| SIGLEC7 | ARHGAP20 | IFI27L1 | BIRC7 | ANGPT4 | CNGA2 |
| MIR4697HG | DPEP1 | CD1E | PRMT8 | LINC02367 | LOC728715 |
| OVOS | TMEM130 | MFF-DT | LRRD1 | MAGEB1 | NXNL1 |
| OR7E47P | FBXW12 | MMD2 | CPLX4 | ANKRD7 | SHISAL2A |
| TRG-AS1 | LINC00485 | NPY2R | PVALB | C7orf33 | C1QB |
| FIBCD1 | LINC02369 | LINC02368 | LINC01257 | LINC02415 | LINC02370 |
| LINC00421 | FGF6 | CHRFAM7A | LINC00428 | ZNF560 | LINC00355 |
| LINC00381 | ATP10B | MASTL | FAM155A-IT1 | ZNF121 | DMRT1 |
| LINC00552 | POTEG | SPOCK1 | SLC22A12 | KCNK13 | SERPINA13P |
| LINC00523 | LSM10 | CACNA11 | MIR381HG | TMEM9 | ORC1 |
| GOLGA8IP | PWAR1 | MYO7A | LINC02249 | DNM1P50 | KNDC1 |
| CMTM5 | HYAL4 | LINC00968 | MYH6 | PCAT29 | UBL7-AS1 |
| SRRM2-AS1 | LOC101927131 | EPS8L3 | RSPH14 | CRX | WT1-AS |
| SUSD3 | LOC101928682 | GABRB1 | VPS9D1-AS1 | IGFN1 | VENTX |
| FSCB | KCNT1 | MLN | RPL29P2 | KRT17P5 | FLJ36000 |
| GOLGA8S | RLBP1 | BNIP5 | FAM153CP | POC1A | KLK11 |
| RSPO4 | USP17L6P | USP17L15 | PTPRT | FHL5 | JAKMIP1 |
| LOC105371925 | GOLGA8Q | SULT2A1 | PRPS2 | GOLGA8J | SLC4A3 |
| CCL25 | GOLGA8T | ABO | SCUBE1 | OBP2A | STK11IP |
| SOHLH1 | LOC101928517 | SIGLEC17P | TRARG1 | SPDYE17 | NTRK1 |
| NLRP13 | LINC01865 | LINC01115 | LINC01304 | LINC00298 | MS4A8 |
| ADGRE1 | LOC339685 | ZNF623 | CRYBB2 | LINC01120 | NOC2LP2 |
| POTEKP | LINC01087 | SPATA31D4 | TPO | FAM153B | SERPINA10 |
| LOC105747689 | FCN2 | MTMR14 | TMEM198 | LOC285097 | PROP1 |
| LOC100289473 | CDC23 | PLK5 | LINC01721 | OTOS | NOL4L-DT |
| TCERG1L | PNPLA1 | LYZL6 | TUBA3C | IZUMO1R | FAM153A |
| CDH4 | LL22NC03- | FAM2301 | IGLL3P | CAD | PRSS35 |
| | 63E9.3 | | | | |
| LOC101927447 | LINC01644 | LINC00898 | ABHD4 | ELP6 | LAMB2P1 |
| CALML6 | MS4A18 | NUP210P1 | LINC01471 | KRT76 | RIMS4 |
| TMEM202 | LINC02010 | DCAF12L1 | KRTAP5-9 | NPTX2 | DPPA2P3 |
| LINC02171 | NT5M | ACTL7B | MTG1 | PCDHB9 | LOC101928131 |
| LINC01262 | LOC105379514 | SSTR3 | EGFLAM-AS2 | HPCAL4 | INSL3 |
| PCDHB5 | NROB2 | LOC644936 | GRIK3 | UBQLNL | PCDHB17P |
| PCDHB18P | PCDHB19P | KRT79 | LOC643201 | CCDC27 | KCNIP1-AS1 |
| DES | KIAA1210 | CASC6 | PNLIPRP1 | UCN3 | WDR64 |
| GABRD | KCNK9 | NPAP1 | KCNK15 | PGA4 | TFAMP1 |
| ANKRD36C | LINC01450 | TMEM89 | LCN9 | SLC25A2 | ZNF516 |
| STAG3L5P- | MT1H | KRT12 | NEU2 | RBBP8NL | LOC100506585 |
| PVRIG2P-PILRB | | | | | |
| TGFBR3L | LOC101929128 | FAM167A-AS1 | LCN15 | LINC01289 | KCNG1 |
| FCRL6 | PKD2L1 | OBP2B | MYH7 | BRK1 | LOC101928160 |
| ZP4 | SEPTIN14 | FAM95C | PAEP | C9orf62 | LOC497256 |
| LOC101927954 | LOC158435 | HSD17B3-AS1 | LINC01492 | NT5C1A | ACTL8 |
| FAM230C | LOC100233156 | LOC389834 | LOC100505874 | GOLGA8K | PCDHB13 |
| MED25 | COL20A1 | KIF2B | LOC101928336 | ARGFX | SRD5A1P1 |
| KRT6A | MYOPARR | PCDHB10 | TTC24 | LRRC18 | DMRTC2 |
| CDHR4 | SPTA1 | ERICH3 | C2orf27B | LCN6 | OR10P1 |
| LRRC70 | SPATA31E1 | LMOD2 | PROK1 | KRT15 | TMPRSS15 |
| MATN3 | CFAP77 | DCAF8L2 | PIRT | SNAP25 | LOC102723971 |

Table2:GenescorrespondingtotheorgansinFIGS.13A-13D.

The genes used for the ageing models are provided in table 3 below. Various embodiments can use all or fewer numbers of genes.

TABLE 3
Genes corresponding to the data used in FIGS. 16-19.

| | | | | | | | |
|---|---|---|---|---|---|---|---|
| ROBO1 | ZIC1 | MEI4 | DSCAM | TRPC4 | NAA11 | CUX2 | MAL2 |
| MUC16 | PLCL1 | NR3C2 | CHRM3 | EVC2 | CLVS2 | ULK1 | WFDC1 |
| DYNC1I1 | NEBL | NTRK3 | MIPOL1 | SLC6A13 | PTCHD4 | FSHR | KIRREL |
| ZNF578 | ATHL1 | ESRRG | ARHGAP42 | SLC36A4 | ZNF365 | DIO2 | PTPRR |
| ITIH2 | IGSF11 | PPARGC1A | VWF | PRR16 | CSN1S1 | SLC8A1 | EPGN |
| RASEF | JPH1 | NKAIN2 | MPDZ | RAC2 | ABCG8 | GABRA4 | THEMIS |
| ADAM22 | VSTM1 | PLAG1 | GPR139 | SLITRK5 | SSTR4 | RYR3 | PRSS27 |
| PKP3 | MLNR | KCNE4 | MEIS2 | ERC2 | TRAF7 | ST8SIA3 | ZMAT4 |
| PRB2 | PLPPR4 | DNAH7 | GAMT | FHOD3 | HMX3 | C12orf50 | GHR |
| GLT6D1 | NPIPB7 | KDR | SERTM1 | GYPB | DUXA | CYP2E1 | ZBTB47 |
| SULF1 | PQLC2L | GPR27 | CD1B | MMP17 | NMUR2 | FTCD | C15orf52 |
| KLF2 | TLR2 | GPATCH2 | F7 | GABRG1 | WASH1 | KHDRBS3 | NBPF1 |
| LAMA2 | BTNL8 | TRAT1 | TMEM176A | ZNF770 | TMTC1 | HS3ST3A1 | LILRB4 |
| LRAT | NTM | ASB5 | LYPD1 | C2orf57 | FRZB | CELA3A | SYT17 |
| ARHGAP44 | ELFN1 | BET1 | PVALB | NBEA | RP11-514P8.7 | ANKRD45 | AL627309.1 |
| RPE65 | RGMB | KCTD12 | TRPM5 | PPP1R17 | FAM171B | FEZF1 | SNCAIP |
| NR2F2 | ANHX | TENM2 | GALNTL6 | ASXL3 | HPSE2 | IER2 | AC017081.1 |
| EFCAB1 | SLC22A10 | PRB1 | RIMBP3C | C11orf87 | ZNF804B | AL162431.1 | ADAM7 |
| TCEA2 | TLX1 | FMO4 | AC092835.2 | IFNA14 | ANXA1 | AC010760.1 | KCNA3 |
| GNG11 | TRIM48 | CAMK2N1 | SMCP | SKOR2 | DDX60 | PTPN20A | PTPN14 |
| CACNA2D3 | ZNF595 | MMP9 | TRIM64C | ZNF43 | UBE2Q2L | DEFB107A | HIST1H4L |
| OXER1 | OR4C46 | SLC5A7 | KRTAP4-9 | ALDH2 | ODAM | FAM27E2 | MUC22 |
| IFI44L | SKOR1 | ZNF90 | CROT | OR6C68 | CYP4V2 | ZNF708 | KRT222 |
| HLA-DQA1 | FGF6 | CPLX2 | PNPLA2 | PSORS1C1 | STK33 | NPAP1 | FAM83H |
| CATSPERB | RIMBP3 | KLRC1 | NDNF | CD33 | SPACA1 | DEFB116 | TBX20 |

The CpG sites used for the ageing models are provided in table 4 below. Various embodiments can use all or fewer numbers of CpG sites.

TABLE 4
CpG sites and correspond genomic locations corresponding
to the data used in FIGS. 16-19.

| Chromosome | Start | End | Gene |
|---|---|---|---|
| chr1 | 16887181 | 16887183 | |
| chr1 | 22470388 | 22470390 | |
| chr1 | 73965483 | 73965485 | |
| chr1 | 149287188 | 149287190 | |
| chr1 | 156186571 | 156186573 | “PMF1, PMF1-BGLAP” |
| chr1 | 162004697 | 162004699 | |
| chr1 | 229046630 | 229046632 | |
| chr11 | 19281463 | 19281465 | |
| chr11 | 44491150 | 44491152 | |
| chr12 | 2505930 | 2505932 | CACNA1C |
| chr12 | 3772411 | 3772413 | |
| chr12 | 13906284 | 13906286 | GRIN2B |
| chr12 | 124710110 | 124710112 | ZNF664-RFLNA |
| chr13 | 22416358 | 22416360 | |
| chr13 | 28502362 | 28502364 | |
| chr14 | 19002252 | 19002254 | |
| chr14 | 36991598 | 36991600 | NKX2-1-AS1 |
| chr15 | 21905657 | 21905659 | |
| chr15 | 28201584 | 28201586 | OCA2 |
| chr15 | 74134548 | 74134550 | |
| chr15 | 93215797 | 93215799 | |
| chr16 | 46396675 | 46396677 | |
| chr16 | 46397548 | 46397550 | |
| chr16 | 46403562 | 46403564 | |
| chr16 | 46421095 | 46421097 | |
| chr17 | 62859683 | 62859685 | LRRC37A3 |
| chr19 | 46995889 | 46995891 | “PNMA8B, PPP5D1” |
| chr20 | 54877643 | 54877645 | |
| chr21 | 11097416 | 11097418 | “BAGE2, BAGE3, BAGE4, |
| | | | BAGE5, BAGE” |
| chr21 | 27998161 | 27998163 | |
| chr3 | 72787599 | 72787601 | |
| chr3 | 129335525 | 129335527 | |
| chr4 | 5438847 | 5438849 | STK32B |
| chr4 | 49514929 | 49514931 | |
| chr4 | 154055423 | 154055425 | |
| chr4 | 184456459 | 184456461 | |
| chr4 | 190496677 | 190496679 | |
| chr4 | 190629494 | 190629496 | |
| chr5 | 69955138 | 69955140 | |
| chr5 | 143229809 | 143229811 | |
| chr5 | 149023834 | 149023836 | |
| chr6 | 17288499 | 17288501 | RBM24 |
| chr7 | 74595499 | 74595501 | |
| chr7 | 120542493 | 120542495 | |
| chr9 | 42717685 | 42717687 | |
| chr9 | 43209115 | 43209117 | |
| chr9 | 69840149 | 69840151 | |
| chr9 | 117264118 | 117264120 | WHRN |
| chr9 | 117537342 | 117537344 | |

### IX. REFERENCES

Cai, J., Chen, L., Zhang, Z., Zhang, X., Lu, X., Liu, W., Shi, G., Ge, Y., Gao, P., and Yang, Y. (2019). Genome-wide mapping of 5-hydroxymethylcytosines in circulating cell-free DNA as a non-invasive approach for early detection of hepatocellular carcinoma. Gut 68, 2195-2205.
Cai, J., Zeng, C., Hua, W., Qi, Z., Song, Y., Lu, X., Li, D., Zhang, Z., Cui, X., and Zhang, X. (2021). An integrative analysis of genome-wide 5-hydroxymethylcytosines in circulating cell-free DNA detects noninvasive diagnostic markers for gliomas. Neuro-oncology advances 3, vdab049.
Fleischer, J. G., Schulte, R., Tsai, H. H., Tyagi, S., Ibarra, A., Shokhirev, M. N., Huang, L., Hetzer, M. W., and Navlakha, S. (2018). Predicting age from the transcriptome of human dermal fibroblasts. Genome biology 19, 1-8.
Greenberg, M. V., and Bourc'his, D. (2019). The diverse roles of DNA methylation in mammalian development and disease. Nature reviews Molecular cell biology 20, 590-607.
Hannum, G., Guinney, J., Zhao, L., Zhang, L., Hughes, G., Sadda, S., Klotzle, B., Bibikova, M., Fan, J.-B., and Gao, Y. (2013). Genome-wide methylation profiles reveal quantitative views of human aging rates. Molecular cell 49, 359-367.
Hertel, J., Friedrich, N., Wittfeld, K., Pietzner, M., Budde, K., Van der Auwera, S., Lohmann, T., Teumer, A., Volzke, H., and Nauck, M. (2016). Measuring biological age via metabonomics: the metabolic age score. Journal of proteome research 15, 400-410.
Horvath, S. (2013). DNA methylation age of human tissues and cell types. Genome biology 14, 1-20.
Koh, K. P., Yabuuchi, A., Rao, S., Huang, Y., Cunniff, K., Nardone, J., Laiho, A., Tahiliani, M., Sommer, C. A., and Mostoslavsky, G. (2011). Tet1 and Tet2 regulate 5-hydroxymethylcytosine production and cell lineage specification in mouse embryonic stem cells. Cell stem cell 8, 200-213.
Kriaucionis, S., and Heintz, N. (2009). The nuclear DNA base 5-hydroxymethylcytosine is present in Purkinje neurons and the brain. Science 324, 929-930.
Lehallier, B., Gate, D., Schaum, N., Nanasi, T., Lee, S. E., Yousef, H., Moran Losada, P., Berdnik, D., Keller, A., and Verghese, J. (2019). Undulating changes in human plasma proteome profiles across the lifespan. Nature medicine 25, 1843-1850.
Leon, S., Shapiro, B., Sklaroff, D., and Yaros, M. (1977). Free DNA in the serum of cancer patients and the effect of therapy. Cancer research 37, 646-650.
Li, J., Xiong, M., Fu, X.-H., Fan, Y., Dong, C., Sun, X., Zheng, F., Wang, S.-W., Liu, L., and Xu, M. (2023). Determining a multimodal aging clock in a cohort of Chinese women. Med 4, 825-848. e813.
Liao, P., Ostrom, Q. T., Stetson, L., and Barnholtz-Sloan, J. S. (2018). Models of epigenetic age capture patterns of DNA methylation in glioma associated with molecular subtype, survival, and recurrence. Neuro-oncology 20, 942-953.
Lin, Q., and Wagner, W. (2015). Epigenetic aging signatures are coherently modified in cancer. PLoS genetics 11, e1005334.
Lister, R., Mukamel, E. A., Nery, J. R., Urich, M., Puddifoot, C. A., Johnson, N. D., Lucero, J., Huang, Y., Dwork, A. J., and Schultz, M. D. (2013). Global epigenomic reconfiguration during mammalian brain development. Science 341, 1237905.
Lo, Y. D., Corbetta, N., Chamberlain, P. F., Rai, V., Sargent, I. L., Redman, C. W., and Wainscoat, J. S. (1997). Presence of fetal DNA in maternal plasma and serum. The lancet 350, 485-487.
Lo, Y. D., Tein, M. S., Pang, C. C., Yeung, C. K., Tong, K.-L., and Hjelm, N. M. (1998). Presence of donor-specific DNA in plasma of kidney and liver-transplant recipients. The Lancet 351, 1329-1330.
Lowsky, D. J., Olshansky, S. J., Bhattacharya, J., and Goldman, D. P. (2014). Heterogeneity in healthy aging. Journals of Gerontology Series A: Biomedical Sciences and Medical Sciences 69, 640-649.
Mandel, P. (1948). Les acides nucleiques du plasma sanguin chez 1 homme. CR Seances Soc Biol Fil 142, 241-243.
Occean, J. R., Yang, N., Sun, Y., Dawkins, M. S., Munk, R., Belair, C., Dar, S., Anerillas, C., Wang, L., and Shi, C. (2023). Gene body DNA hydroxymethylation restricts the magnitude of transcriptional changes during aging. bioRxiv.
Oh, H. S.-H., Rutledge, J., Nachun, D., Pilovics, R., Abiose, O., Moran-Losada, P., Channappa, D., Urey, D. Y., Kim, K., and Sung, Y. J. (2023). Organ aging signatures in the plasma proteome track health and disease. Nature 624, 164-172.
Partridge, L., Deelen, J., and Slagboom, P. E. (2018). Facing up to the global challenges of ageing. Nature 561, 45-56.
Pastor, W. A., Pape, U. J., Huang, Y., Henderson, H. R., Lister, R., Ko, M., McLoughlin, E. M., Brudno, Y., Mahapatra, S., and Kapranov, P. (2011). Genome-wide mapping of 5-hydroxymethylcytosine in embryonic stem cells. Nature 473, 394-397.
Peters, M. J., Joehanes, R., Pilling, L. C., Schurmann, C., Conneely, K. N., Powell, J., Reinmaa, E., Sutphin, G. L., Zhernakova, A., and Schramm, K. (2015). The transcriptional landscape of age in human peripheral blood. Nature communications 6, 1-14.
Robinson, O., Chadeau Hyam, M., Karaman, I., Climaco Pinto, R., Ala-Korpela, M., Handakas, E., Fiorito, G., Gao, H., Heard, A., and Jarvelin, M. R. (2020). Determinants of accelerated metabolomic and epigenetic aging in a UK cohort. Aging Cell 19, e13149.
Rutledge, J., Oh, H., and Wyss-Coray, T. (2022). Measuring biological age using omics data. Nature Reviews Genetics 23, 715-727.
Satija, R., Farrell, J. A., Gennert, D., Schier, A. F., and Regev, A. (2015). Spatial reconstruction of single-cell gene expression data. Nature biotechnology 33, 495-502.
Shao, J., Bernicker, E. H., He, C., and Li, Z. (2022). Cell-free DNA 5-hydroxymethylcytosine as a marker for common cancer detection. Clinical and Translational Discovery 2, e136.
Szulwach, K. E., Li, X., Li, Y., Song, C.-X., Wu, H., Dai, Q., Irier, H., Upadhyay, A. K., Gearing, M., and Levey, A. I. (2011). 5-hmC-mediated epigenetic dynamics during postnatal neurodevelopment and aging. Nature neuroscience 14, 1607-1616.
Walker, N. J., Rashid, M., Yu, S., Bignell, H., Lumby, C. K., Livi, C. M., Howell, K., Morley, D. J., Morganella, S., and Barrell, D. (2022). Hydroxymethylation profile of cell-free DNA is a biomarker for early colorectal cancer. Scientific Reports 12, 16566.
Xiong, J., Jiang, H.-P., Peng, C.-Y., Deng, Q.-Y., Lan, M.-D., Zeng, H., Zheng, F., Feng, Y.-Q., and Yuan, B.-F. (2015). DNA hydroxymethylation age of human blood determined by capillary hydrophilic-interaction liquid chromatography/mass spectrometry. Clinical epigenetics 7, 1-11.

1. A method for measuring a biological age of a subject, the method comprising performing by a computer system:
receiving sequence reads including methylation statuses at sites of a plurality of cell-free DNA molecules;
aligning the sequence reads to a reference genome, wherein the sequence reads are aligned to N sets of one or more CpG sites;
for each set of the N sets of one or more CpG sites:
identifying a group of sequence reads aligning to the set of one or more CpG sites in the reference genome; and
determining a methylation level using the methylation statuses of the group of sequence reads, wherein N is at least 3, thereby determining N methylation levels;
generating a feature vector from the N methylation levels;
loading a machine learning model into memory of the computer system, the machine learning model being trained using training samples having a known chronological age and measured reference vectors of methylation levels;
inputting the feature vector into the machine learning model; and
predicting, using the machine learning model, the biological age of the subject.

2. The method of claim 1, wherein the N sets of one or more CpG sites are associated with a particular tissue type, and wherein the biological age is for the particular tissue type.

3. The method of claim 2, wherein the N sets of one or more CpG sites are associated with the particular tissue type based on (1) a biological pathway, (2) epigenetic patterns, or (3) expression levels in the particular tissue type being greater than a threshold.

4. The method of claim 2, wherein the particular tissue type is for a particular organ.

5. The method of claim 2, wherein the particular tissue type is selected from table 2, and wherein the N sets of one or more CpG sites are selected from the genes listed as associated with the particular tissue type in table 2.

6. The method of claim 2, wherein the particular tissue type is selected from a group consisting of: bone marrow, brain, ovary, pancreas, liver, hypothalamus, heart, kidney, bladder, prostate, lymph nodes, breast, lung, skin, and testis.

7. The method of claim 1, wherein the biological age is an age range.

8. The method of claim 1, wherein the machine learning model is a regression model.

9. The method of claim 1, wherein determining the methylation level at the set of one or more CpG sites includes determining an amount of the methylation statuses at the one or more CpG sites that indicate a methylation is present or that indicate the methylation is not present.

10. The method of claim 9, wherein the methylation level is a methylation density.

11. The method of claim 9, wherein the methylation level includes a proportion of the methylation statuses at the sites that indicate the methylation is present or that indicate the methylation is not present.

12. The method of claim 1, wherein the N sets of one or more CpG sites correspond to N genes, and wherein the N methylation levels are N gene-specific methylation levels.

13. The method of claim 12, wherein the N gene-specific methylation levels are of 5hmC.

14. A method for detecting a pathology in a subject having a known chronological age, the method comprising performing by a computer system:
receiving sequence reads including methylation statuses at sites of a plurality of cell-free DNA molecules;
aligning the sequence reads to a reference genome, wherein the sequence reads are aligned to N sets of one or more CpG sites;
for each set of the N sets of one or more CpG sites:
identifying a group of sequence reads aligning to the set of one or more CpG sites in the reference genome; and
determining a methylation level using the methylation statuses of the group of sequence reads, wherein N is at least 3, thereby determining N methylation levels;
generating a feature vector from the N methylation levels;
loading an age-dependent machine learning model into memory of the computer system, the age-dependent machine learning model being trained using training samples having the known chronological age, known pathology classifications, and measured reference vectors of methylation levels;
inputting the feature vector into the age-dependent machine learning model; and
determining, by the age-dependent machine learning model using the feature vector, a classification of a presence of the pathology in the subject.

15. The method of claim 14, wherein the known chronological age is an age range.

16. The method of claim 14, wherein the age-dependent machine learning model includes a plurality of sub-models, each corresponding to a different chronological age.

17. The method of claim 14, wherein determining the classification of the presence of the pathology in the subject includes:
comparing the feature vector to a representative reference vector determined using a group of the measured reference vectors that have a same known pathology classification.

18. The method of claim 14, wherein determining the classification of the presence of the pathology in the subject includes:
predicting, using the age-dependent machine learning model, a biological age of the subject;
comparing the biological age to the known chronological age; and
determining the classification of the presence of the pathology in the subject based on the comparison.

19. The method of claim 18, wherein determining the classification of the presence of the pathology in the subject further includes:
determining a difference between the biological age and the known chronological age; and
comparing the difference to a threshold to determine the classification of the presence of the pathology in the subject.

20. The method of claim 14, wherein the N sets of one or more CpG sites are associated with a particular tissue type, and wherein the pathology is for the particular tissue type.

21. The method of claim 20, wherein the N sets of one or more CpG sites are associated with the particular tissue type based on (1) a biological pathway or (2) epigenetic patterns or (3) expression levels in the particular tissue type being greater than a threshold.

22. The method of claim 20, wherein the particular tissue type is for a particular organ.

23. The method of claim 20, wherein the particular tissue type is selected from table 2, and wherein the N sets of CpG sites are selected from the genes listed as associated with the particular tissue type in table 2.

24. The method of claim 20, wherein the particular tissue type is selected from a group consisting of: bone marrow, brain, ovary, pancreas, liver, hypothalamus, heart, kidney, bladder, prostate, lymph nodes, breast, lung, skin, and testis.

25. The method of claim 14, wherein determining the methylation level at the set of one or more CpG sites includes determining an amount of the methylation statuses at the one or more CpG sites that indicate a methylation is present or that indicate the methylation is not present.

26. The method of claim 25, wherein the methylation level is a methylation density.

27. The method of claim 25, wherein the methylation level includes a proportion of the methylation statuses at the sites that indicate the methylation is present or that indicate the methylation is not present.

28. The method of claim 14, wherein the N sets of one or more CpG sites correspond to N genes, and wherein the N methylation levels are N gene-specific methylation levels.

29. The method of claim 28, wherein the N gene-specific methylation levels are of 5hmC.

30. A method for detecting a pathology in a subject having a known chronological age, the method comprising performing by a computer system:
receiving sequence reads including methylation statuses at sites of a plurality of cell-free DNA molecules;
aligning the sequence reads to a reference genome;
for each group of one or more groups of sets of CpG sites:
identifying a group of sequence reads aligning to any CpG site in the group of sets of CpG sites, the group of sets of CpG sites including at least 3 sets of CpG sites, wherein each set of CpG sites in the group has a same shape classification for a change in a methylation level with respect to age; and
determining one or more methylation levels using the methylation statuses of the group of sequence reads; and
determining, using a model that varies with age, a classification of a presence of the pathology in the subject, wherein the determining uses the known chronological age of the subject and the one or more methylation levels, and wherein the model is generated using reference samples of subjects having known classifications for the pathology.

31. The method of claim 30, wherein a first group of the one or more groups of sets of CpG sites corresponds to one or more genes, and wherein the one or more genes includes a cluster in table 1.

32. The method of claim 30, wherein determining, using the model, the classification includes comparing the one or more methylation levels to one or more thresholds, wherein the one or more thresholds are dependent on the known chronological age.

33. The method of claim 30, wherein the model is an age-dependent machine learning model.

34. The method of claim 33, wherein the age-dependent machine learning model includes a plurality of sub-models, each corresponding to a different chronological age.

35. The method of claim 33, wherein the one or more groups of sets of CpG sites is a plurality of groups of sets of CpG sites, and wherein determining the classification of the presence of the pathology in the subject includes:
generating a feature vector from the one or more methylation levels of the plurality of groups of sets of CpG sites; and
comparing the feature vector to a representative reference vector determined using a group of measured reference vectors that have a same known pathology classification.

36. The method of claim 33, wherein determining the classification of the presence of the pathology in the subject includes:
predicting, using the age-dependent machine learning model, a biological age of the subject;
comparing the biological age to the known chronological age; and
determining the classification of the presence of the pathology in the subject based on the comparison.

37. The method of claim 36, wherein determining the classification of the presence of the pathology in the subject further includes:
determining a difference between the biological age and the known chronological age; and
comparing the difference to a threshold to determine the classification of the presence of the pathology in the subject.

38. The method of claim 30, wherein the same shape classification is selected from a group consisting of linear, logarithmic, quadratic, and exponential.

39. The method of claim 30, wherein each group of the one or more groups of sets of CpG sites is associated with a particular tissue type, and wherein the pathology is for the particular tissue type.

40. The method of claim 39, wherein each group of the one or more groups of sets of CpG sites are associated with the particular tissue type based on (1) a biological pathway or (2) epigenetic patterns or (3) expression levels in the particular tissue type being greater than a threshold.

41. The method of claim 39, wherein the particular tissue type is for a particular organ.

42. The method of claim 39, wherein the particular tissue type is selected from table 2, and wherein the one or more groups of sets of CpG sites are selected from the genes listed as associated with the particular tissue type in table 2.

43. The method of claim 39, wherein the particular tissue type is selected from a group consisting of: bone marrow, brain, ovary, pancreas, liver, hypothalamus, heart, kidney, bladder, prostate, lymph nodes, breast, lung, skin, and testis.

44. The method of claim 30, wherein the pathology is a tumor.

45. The method of claim 44, wherein the pathology is Glioma.

46. The method of claim 1, wherein the sequence reads are determined using sequencing or probe-based techniques.

47. The method of claim 46, wherein the sequencing includes determining the methylation status by (1) treating the plurality of cell-free DNA molecules (e.g., with bisulfite or a restriction enzyme) or (2) analyzing optical or electrical signals of the plurality of cell-free DNA molecules at positions within a window that includes the site.

48. The method of claim 1, wherein the sequence reads are paired-end reads.

49. The method of claim 30, wherein determining the methylation level the group of sets of CpG sites includes determining an amount of the methylation statuses at the sets of CpG sites that indicates that a methylation is present or that indicates that the methylation is not present.

50. The method of claim 49, wherein the methylation level is a methylation density.

51. The method of claim 49, wherein the methylation level includes a proportion of the methylation statuses at the sets of CpG sites that indicate the methylation is present or that indicate the methylation is not present.

52. The method of claim 1, wherein the one or more groups of sets of CpG sites correspond to one or more genes, and wherein the methylation levels are gene-specific methylation levels.

53-57. (canceled)
